SEQUENCE LISTING (157 sequences)

SEQ ID NO: 1 - protein, 1231 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVKTCSKSSIDIENGFISESQYTYALKEKAKYQCKLGYVTADGETSGSITCGKDGWSAQPTCIKSCDIPVFMNARTKNDFTWFKLNDTLDYECHDGYESNTGSTTGSIVCGYNGWSDLPICYERECELPKIDVHLVPDRKKDQYKVGEVLKFSCKPGFTIVGPNSVQCYHFGLSPDLPICKEQVQSCGPPPELLNGNVKEKTKEEYGHSEVVEYYCNPRFLMKGPNKIQCVDGEWTTLPVCIVEESTCGDIPELEHGWAQLSSPPYYYGDSVEFNCSESFTMIGHRSITCIHGVWTQLPQCVAIDKLKKCKSSNLIILEEHLKNKKEFDHNSNIRYRCRGKEGWIHTVCINGRWDPEVNCSMAQIQLCPPPPQIPNSHNMTTTLNYRDGEKVSVLCQENYLIQEGEEITCKDGRWQSIPLCVEKIPCSQPPQIEHGTINSSRSSQESYAHGTKLSYTCEGGFRISEENETTCYMGKWSSPPQCEGLPCKSPPEISHGVVAHMSDSYQYGEEVTYKCFEGFGIDGPAIAKCLGEKWSHPPSCIKTDCLSLPSFENAIPMGEKKDVYKAGEQVTYTCATYYKMDGASNVTCINSRWTGRPTCRDTSCVNPPTVQNAYIVSRQMSKYPSGERVRYQCRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYSRTGESVEFVCKRGYRLSSRSHTLRTTCWDGKLEYPTCAKR

SEQ ID NO: 2 - protein, 449 aa, Homo sapiens
MRLLAKIICLMLWAICVAEDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEEKSCDNPYIPNGDYSPLRIKHRTGDEITYQCRNGFYPATRGNTAKCTSTGWIPAPRCTLKPCDYPDIKHGGLYHENMRRPYFPVAVGKYYSYYCDEHFETPSGSYWDHIHCTQDGWSPAVPCLRKCYFPYLENGYNQNYGRKFVQGKSIDVACHPGYALPKAQTTVTCMENGWSPTPRCIRVSFTL

SEQ ID NO: 3 - protein, 330 aa, Homo sapiens
MWLLVSVILISRISSVGGEATFCDFPKINHGILYDEEKYKPFSQVPTGEVFYYSCEYNFVSPSKSFWTRITCTEEGWSPTPKCLRLCFFPFVENGHSESSGQTHLEGDTVQIICNTGYRLQNNENNISCVERGWSTPPKCRSTDTSCVNPPTVQNAHILSRQMSKYPSGERVRYECRSPYEMFGDEEVMCLNGNWTEPPQCKDSTGKCGPPPPIDNGDITSFPLSVYAPASSVEYQCQNLYQLEGNKRITCRNGQWSEPPKCLHPCVISREIMENYNIALRWTAKQKLYLRTGESAEFVCKRGYRLSSRSHTLRTTCWDGKLEYPTCAKR

SEQ ID NO: 4 - protein, 270 aa, Homo sapiens
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTISAEKCGPPPPIDNGDITSFLLSVYAPGSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 5 - protein, 243 aa, Homo sapiens
MWLLVSVILISRISSVGGEAMFCDFPKINHGILYDEEKYKPFSQVPTGEVFYYSCEYNFVSPSKSFWTRITCAEEGWSPTPKCLRLCFFPFVENGHSESSGQTHLEGDTVQIICNTGYRLQNNENNISCVERGWSTPPKCRSTSSSVEYQCQNLYQLEGNNQITCRNGQWSEPPKCLDPCVISQEIMEKYNIKLKWTNQQKLYSRTGDIVEFVCKSGYHPTKSHSFRAMCQNGKLVYPSCEEK

SEQ ID NO: 6 - protein, 330 aa, Homo sapiens
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVRTCSKSDIEIENGFISESSSIYILNKEIQYKCKPGYATADGNSSGSITCLQNGWSAQPICINSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 7 - protein, 269 aa, Homo sapiens
MLLLINVILTLWVSCANGQVKPCDFPDIKHGGLFHENMRRPYFPVAVGKYYSYYCDEHFETPSGSYWDYIHCTQNGWSPAVPCLRKCYFPYLENGYNQNYGRKFVQGNSTEVACHPGYGLPKAQTTVTCTEKGWSPTPRCIRVNSSEKCGPPPPISNGDTTSFLLKVYVPQSRVEYQCQPYYELQGSNYVTCSNGEWSEPPRCIHPCIITEENMNKNNIKLKGRSDRKYYAKTGDTIEFMCKLGYNANTSILSFQAVCREGIVEYPRCE

SEQ ID NO: 8 - protein, 578 aa, Homo sapiens
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDVEIENGFISESSSIYILNEETQYNCKPGYATAEGNSSGSITCLQNGWSTQPICIKFCDMPVFENSRAKSNGMWFKLHDTLDYECYDGYESSYGNTTDSIVCGEDGWSHLPTCYNSSENCGPPPPISNGDTTSFPQKVYLPWSRVEYQCQSYYELQGSKYVTCSNGDWSEPPRCISMKPCEFPEIQHGHLYYENTRRPYFPVATGQSYSYYCDQNFVTPSGSYWDYIHCTQDGWLPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 9 - protein, 331 aa, Homo sapiens
MLLLINVILTLWVSCANGQEVKPCDFPEIQHGGLYYKSLRRLYFPAAAGQSYSYYCDQNFVTPSGSYWDYIHCTQDGWSPTVPCLRTCSKSDIEIENGFISESSSIYILNKEIQYKCKPGYATADGNSSGSITCLQNGWSAQPICIKFCDMPVFENSRAKSNGMRFKLHDTLDYECYDGYEISYGNTTGSIVCGEDGWSHFPTCYNSSEKCGPPPPISNGDTTSFLLKVYVPQSRVEYQCQSYYELQGSNYVTCSNGEWSEPPRCIHPCIITEENMNKNNIQLKGKSDIKYYAKTGDTIEFMCKLGYNANTSVLSFQAVCREGIVEYPRCE

SEQ ID NO: 10 - protein, 569 aa, Homo sapiens
MLLLFSVILISWVSTVGGEGTLCDFPKIHHGFLYDEEDYNPFSQVPTGEVFYYSCEYNFVSPSKSFWTRITCTEEGWSPTPKCLRMCSFPFVKNGHSESSGLIHLEGDTVQIICNTGYSLQNNEKNISCVERGWSTPPICSFTKGECHVPILEANVDAQPKKESYKVGDVLKFSCRKNLIRVGSDSVQCYQFGWSPNFPTCKGQVRSCGPPPQLSNGEVKEIRKEEYGHNEVVEYDCNPNFIINGPKKIQCVDGEWTTLPTCVEQVKTCGYIPELEYGYVQPSVPPYQHGVSVEVNCRNEYAMIGNNMITCINGIWTELPMCVATHQLKRCKIAGVNIKTLLKLSGKEFNHNSRIRYRCSDIFRYRHSVCINGKWNPEVDCTEKREQFCPPPPQIPNAQNMTTTVNYQDGEKVAVLCKENYLLPEAKEIVCKDGRWQSLPRCVESTAYCGPPPSINNGDTTSFPLSVYPPGSTVTYRCQSFYKLQGSVTVTCRNKQWSEPPRCLDPCVVSEENMNKNNIQLKWRNDGKLYAKTGDAVEFQCKFPHKAMISSPPFRAICQEGKFEYPICE

SEQ ID NO: 11 - protein, 583 aa, Homo sapiens
MKLLHVFLLFLCFHLRFCKVTYTSQEDLVEKKCLAKKYTHLSCDKVFCQPWQRCIEGTCVCKLPYQCPKNGTAVCATNRRSFPTYCQQKSLECLHPGTKFLNNGTCTAEGKFSVSLKHGNTDSEGIVEVKLVDQDKTMFICKSSWSMREANVACLDLGFQQGADTQRRFKLSDLSINSTECLHVHCRGLETSLAECTFTKRRTMGYQDFADVVCYTQKADSPMDDFFQCVNGKYISQMKACDGINDCGDQSDELCCKACQGKGFHCKSGVCIPSQYQCNGEVDCITGEDEVGCAGFASVTQEETEILTADMDAERRRIKSLLPKLSCGVKNRMHIRRKRIVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVGRPFISQYNV

SEQ ID NO: 12 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 13 - protein, 645 aa, Homo sapiens
SPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAA

SEQ ID NO: 14 - protein, 915 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 15 - protein, 77 aa, Homo sapiens
SVQLTEKRMDKVGKYPKELRKCCEDGMRENPMRFSCQRRTRFISLGEACKKVFLDCCNYITELRRQHARASHLGLAR

SEQ ID NO: 16 - protein, 915 aa, Homo sapiens; identical to SEQ ID NO: 14

SEQ ID NO: 17 - protein, 343 aa, Homo sapiens
SEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 18 - protein, 17 aa, Homo sapiens
SSKITHRIHWESASLLR

SEQ ID NO: 19 - protein, 206 aa, Homo sapiens
SNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGR

SEQ ID NO: 20 - protein, 7 aa, Homo sapiens
VTYKCFE

SEQ ID NO: 21 - protein, 16 aa, Homo sapiens
NGWSPTPRCIRVSFTL

SEQ ID NO: 22 - protein, 18 aa, Homo sapiens
ATFCDFPKINHGILYDEE

SEQ ID NO: 23 - protein, 16 aa, Homo sapiens
RGWSTPPKCRSTISAE

SEQ ID NO: 24 - protein, 18 aa, Homo sapiens
AMFCDFPKINHGILYDEE

SEQ ID NO: 25 - protein, 20 aa, Homo sapiens
VACHPGYGLPKAQTTVTCTE

SEQ ID NO: 26 - protein, 8 aa, Homo sapiens
YQCQSYYE

SEQ ID NO: 27 - protein, 15 aa, Homo sapiens
RGWSTPPICSFTKGE

SEQ ID NO: 28 - protein, 13 aa, Homo sapiens
GTAFVIFGIQDGE

SEQ ID NO: 29 - protein, 20 aa, Homo sapiens
LRRQHARASHLGLARSNLDE

SEQ ID NO: 30 - protein, 15 aa, Homo sapiens
LRRQHARASHLGLAR

SEQ ID NO: 31 - protein, 23 aa, Homo sapiens
LNLDVSLQLPSRSSKITHRIHWE

SEQ ID NO: 32 - protein, 12 aa, Homo sapiens
LNLDVSLQLPSR

SEQ ID NO: 33 - protein, 5 aa, Homo sapiens
RLGRE

SEQ ID NO: 34 - protein, 11 aa, Homo sapiens
SSKITHRIHWE

SEQ ID NO: 35 - protein, 6 aa, Homo sapiens
SASLLR

SEQ ID NO: 36 - protein, 4 aa, Homo sapiens
RLGR

SEQ ID NO: 37 - protein, 11 aa, Homo sapiens
HLIVTPSGCGE

SEQ ID NO: 38 - protein, 39 aa, Homo sapiens
CAGTYDGSIDACKGDSGGPLVCMDANNVTYVWGVVSWGE

SEQ ID NO: 39 - protein, 36 aa, Homo sapiens
GTCVCKLPYQCPKNGTAVCATNRRSFPTYCQQKSLE

SEQ ID NO: 40 - protein, 29 aa, Homo sapiens
FPGVYTKVANYFDWISYHVGRPFISQYNV

SEQ ID NO: 41 - protein, 31 aa, Homo sapiens
ANVACLDLGFQQGADTQRRFKLSDLSINSTE

SEQ ID NO: 42 - protein, 30 aa, Homo sapiens
LPRSIPACVPWSPYLFQPNDTCIVSGWGRE

SEQ ID NO: 43 - protein, 26 aa, Homo sapiens
KKCLAKKYTHLSCDKVFCQPWQRCIE

SEQ ID NO: 44 - protein, 28 aa, Homo sapiens
LCCKACQGKGFHCKSGVCIPSQYQCNGE

SEQ ID NO: 45 - protein, 21 aa, Homo sapiens
VKLVDQDKTMFICKSSWSMRE

SEQ ID NO: 46 - protein, 17 aa, Homo sapiens
VKLISNCSKFYGNRFYE

SEQ ID NO: 47 - protein, 17 aa, Homo sapiens
CLHPGTKFLNNGTCTAE

SEQ ID NO: 48 - protein, 15 aa, Homo sapiens
NYNAGTYQNDIALIE

SEQ ID NO: 49 - protein, 15 aa, Homo sapiens
GKFSVSLKHGNTDSE

SEQ ID NO: 50 - protein, 13 aa, Homo sapiens
VGCAGFASVTQEE

SEQ ID NO: 51 - protein, 11 aa, Homo sapiens
MKKDGNKKDCE

SEQ ID NO: 52 - protein, 9 aa, Homo sapiens
YVDRIIFHE

SEQ ID NO: 53 - protein, 10 aa, Homo sapiens
CLHVHCRGLE

SEQ ID NO: 54 - protein, 9 aa, Homo sapiens
RVFSLQWGE

SEQ ID NO: 55 - protein, 9 aa, Homo sapiens
ILTADMDAE

SEQ ID NO: 56 - protein, 8 aa, Homo sapiens
KVTYTSQE

SEQ ID NO: 57 - protein, 7 aa, Homo sapiens
VDCITGE

SEQ ID NO: 58 - protein, 6 aa, Homo sapiens
NCGKPE

SEQ ID NO: 59 - protein, 5 aa, Homo sapiens
TSLAE

SEQ ID NO: 60 - protein, 4 aa, Homo sapiens
KDNE

SEQ ID NO: 61 - protein, 61 aa, Homo sapiens
AKIRYYTYLIMNKGRLLKAGRQVREPGQDLVVLPLSITTDFIPSFRLVAYYTLIGASGQRE

SEQ ID NO: 62 - protein, 52 aa, Homo sapiens
RSGIPIVTSPYQIHFTKTPKYFKPGMPFDLMVFVTNPDGSPAYRVPVAVQGE

SEQ ID NO: 63 - protein, 50 aa, Homo sapiens
KVVLVSLQSGYLFIQTDKTIYTPGSTVLYRIFTVNHKLLPVGRTVMVNIE

SEQ ID NO: 64 - protein, 36 aa, Homo sapiens
DTVQSLTQGDGVAKLSINTHPSQKPLSITVRTKKQE

SEQ ID NO: 65 - protein, 35 aa, Homo sapiens
GDHGARVVLVAVDKGVFVLNKKNKLTQSKIWDVVE

SEQ ID NO: 66 - protein, 38 aa, Homo sapiens
KADIGCTPGSGKDYAGVFSDAGLTFTSSSGQQTAQRAE

SEQ ID NO: 67 - protein, 29 aa, Homo sapiens
QATRTMQALPYSTVGNSNNYLHLSVLRTE

SEQ ID NO: 68 - protein, 27 aa, Homo sapiens
AHDAQGDVPVTVTVHDFPGKKLVLSSE

SEQ ID NO: 69 - protein, 26 aa, Homo sapiens
GIPVKQDSLSSQNQLGVLPLSWDIPE

SEQ ID NO: 70 - protein, 26 aa, Homo sapiens
VVADSVWVDVKDSCVGSLVVKSGQSE

SEQ ID NO: 71 - protein, 25 aa, Homo sapiens
DLVGKSLYVSATVILHSGSDMVQAE

SEQ ID NO: 72 - protein, 23 aa, Homo sapiens
KTVLTPATNHMGNVTFTIPANRE

SEQ ID NO: 73 - protein, 19 aa, Homo sapiens
KGRNKFVTVQATFGTQVVE

SEQ ID NO: 74 - protein, 18 aa, Homo sapiens
VVLSRKVLLDGVQNPRAE

SEQ ID NO: 75 - protein, 15 aa, Homo sapiens
TLNVNFLLRMDRAHE

SEQ ID NO: 76 - protein, 14 aa, Homo sapiens
LVNMGQWKIRAYYE

SEQ ID NO: 77 - protein, 15 aa, Homo sapiens
SPMYSIITPNILRLE

SEQ ID NO: 78 - protein, 15 aa, Homo sapiens
DRQPVPGQQMTLKIE

SEQ ID NO: 79 - protein, 14 aa, Homo sapiens
VTITARFLYGKKVE

SEQ ID NO: 80 - protein, 13 aa, Homo sapiens
GTAFVIFGIQDGE

SEQ ID NO: 81 - protein, 8 aa, Homo sapiens
KFYYIYNE

SEQ ID NO: 82 - protein, 10 aa, Homo sapiens
NSPQQVFSTE

SEQ ID NO: 83 - protein, 8 aa, Homo sapiens
SLKRIPIE

SEQ ID NO: 84 - protein, 7 aa, Homo sapiens
YVLPSFE

SEQ ID NO: 85 - protein, 7 aa, Homo sapiens
QRISLPE

SEQ ID NO: 86 - protein, 8 aa, Homo sapiens
LQCPQPAA

SEQ ID NO: 87 - protein, 7 aa, Homo sapiens
VIVEPTE

SEQ ID NO: 88 - protein, 5 aa, Homo sapiens
TMVLE

SEQ ID NO: 89 - protein, 5 aa, Homo sapiens
LRPGE

SEQ ID NO: 90 - protein, 4 aa, Homo sapiens
FKSE

SEQ ID NO: 91 - protein, 20 aa, Homo sapiens
NYNIALRWTAKQKLYLRTGE

SEQ ID NO: 92 - protein, 19 aa, Homo sapiens
YNFVSPSKSFWTRITCAEE

SEQ ID NO: 93 - protein, 21 aa, Homo sapiens
KGWSPTPRCIRVRTCSKSDIE

SEQ ID NO: 94 - protein, 18 aa, Homo sapiens
NGYNQNYGRKFVQGNSTE

SEQ ID NO: 95 - protein, 18 aa, Homo sapiens
QVKPCDFPDIKHGGLFHE

SEQ ID NO: 96 - protein, 22 aa, Homo sapiens
FMCKLGYNANTSILSFQAVCRE

SEQ ID NO: 97 - protein, 8 aa, Homo sapiens
YQCQPYYE

SEQ ID NO: 98 - protein, 20 aa, Homo sapiens
NSRAKSNGMRFKLHDTLDYE

SEQ ID NO: 99 - protein, 14 aa, Homo sapiens
DGWSHFPTCYNSSE

SEQ ID NO: 100 - protein, 14 aa, Homo sapiens
ISYGNTTGSIVCGE

SEQ ID NO: 101 - protein, 22 aa, Homo sapiens
FMCKLGYNANTSVLSFQAVCRE

SEQ ID NO: 102 - protein, 18 aa, Homo sapiens
GTLCDFPKIHHGFLYDEE

SEQ ID NO: 103 - protein, 18 aa, Homo sapiens
YAMIGNNMITCINGIWTE

SEQ ID NO: 104 - protein, 18 aa, Homo sapiens
YGYVQPSVPPYQHGVSVE

SEQ ID NO: 105 - protein, 18 aa, Homo sapiens
GDTVQIICNTGYSLQNNE

SEQ ID NO: 106 - protein, 16 aa, Homo sapiens
IVCKDGRWQSLPRCVE

SEQ ID NO: 107 - protein, 12 aa, Homo sapiens
DYNPFSQVPTGE

SEQ ID NO: 108 - protein, 10 aa, Homo sapiens
QVKTCGYIPE

SEQ ID NO: 109 - protein, 10 aa, Homo sapiens
ANVDAQPKKE

SEQ ID NO: 110 - protein, 9 aa, Homo sapiens
WTTLPTCVE

SEQ ID NO: 111 - protein, 8 aa, Homo sapiens
KVAVLCKE

SEQ ID NO: 112 - protein, 25 aa, Homo sapiens
SNTGSTTGSIVCGYNGWSDLPICYE

SEQ ID NO: 113 - protein, 23 aa, Homo sapiens
NGWSPTPRCIRVKTCSKSSIDIE

SEQ ID NO: 114 - protein, 21 aa, Homo sapiens
LPKIDVHLVPDRKKDQYKVGE

SEQ ID NO: 115 - protein, 21 aa, Homo sapiens
YYCNPRFLMKGPNKIQCVDGE

SEQ ID NO: 116 - protein, 20 aa, Homo sapiens
NYNIALRWTAKQKLYSRTGE

SEQ ID NO: 117 - protein, 20 aa, Homo sapiens
KWSHPPSCIKTDCLSLPSFE

SEQ ID NO: 118 - protein, 18 aa, Homo sapiens
HGWAQLSSPPYYYGDSVE

SEQ ID NO: 119 - protein, 18 aa, Homo sapiens
ISHGVVAHMSDSYQYGEE

SEQ ID NO: 120 - protein, 15 aa, Homo sapiens
FDHNSNIRYRCRGKE

SEQ ID NO: 121 - protein, 16 aa, Homo sapiens
ITCKDGRWQSIPLCVE

SEQ ID NO: 122 - protein, 15 aa, Homo sapiens
GWIHTVCINGRWDPE

SEQ ID NO: 123 - protein, 16 aa, Homo sapiens
KAKYQCKLGYVTADGE

SEQ ID NO: 124 - protein, 15 aa, Homo sapiens
TTCYMGKWSSPPQCE

SEQ ID NO: 125 - protein, 13 aa, Homo sapiens
SYAHGTKLSYTCE

SEQ ID NO: 126 - protein, 11 aa, Homo sapiens
RVRYQCRSPYE

SEQ ID NO: 127 - protein, 15 aa, Homo sapiens
GFGIDGPAIAKCLGE

SEQ ID NO: 128 - protein, 12 aa, Homo sapiens
HGTINSSRSSQE

SEQ ID NO: 129 - protein, 10 aa, Homo sapiens
YQCQNLYQLE

SEQ ID NO: 130 - protein, 11 aa, Homo sapiens
WTTLPVCIVEE

SEQ ID NO: 131 - protein, 11 aa, Homo sapiens
KIPCSQPPQIE

SEQ ID NO: 132 - protein, 9 aa, Homo sapiens
SQYTYALKE

SEQ ID NO: 133 - protein, 10 aa, Homo sapiens
QVQSCGPPPE

SEQ ID NO: 134 - protein, 9 aa, Homo sapiens
KKDVYKAGE

SEQ ID NO: 135 - protein, 9 aa, Homo sapiens
GLPCKSPPE

SEQ ID NO: 136 - protein, 8 aa, Homo sapiens
KVSVLCQE

SEQ ID NO: 137 - protein, 7 aa, Homo sapiens
HLKNKKE

SEQ ID NO: 138 - protein, 8 aa, Homo sapiens
GGFRISEE

SEQ ID NO: 139 - protein, 8 aa, Homo sapiens
LLNGNVKE

SEQ ID NO: 140 - protein, 7 aa, Homo sapiens
YPTCAKR

SEQ ID NO: 141 - protein, 8 aa, Homo sapiens
STCGDIPE

SEQ ID NO: 142 - protein, 349 aa, Homo sapiens
EGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 143 - protein, 47 aa, Homo sapiens
EGVQKEDIPPADLSDQVPDTESETRILLQGTPVAQMTEDAVDAERLK

SEQ ID NO: 144 - protein, 302 aa, Homo sapiens
HLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSR

SEQ ID NO: 145 - protein, 194 aa, Homo sapiens
  SITE 111: note = misc_feature - Xaa can be any naturally occurring amino acid
  SITE 157: note = misc_feature - Xaa can be any naturally occurring amino acid
  SITE 160: note = misc_feature - Xaa can be any naturally occurring amino acid
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNXAHWSTKPPICQRIPCGLPPTIANGDFISTNRENFHYGSVVTYRCNXGSXGRKVFELVGEPSIYCTSNDDQVGIWSGPAPQCII

SEQ ID NO: 146 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCII

SEQ ID NO: 147 - protein, 194 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 148 - protein, 388 aa, Homo sapiens
GHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNAAHWSTKPPICQRIPCGLPPTIANGDFISTNRENFHYGSVVTYRCNPGSGGRKVFELVGEPSIYCTSNDDQVGIWSGPAPQCIIGHCQAPDHFLFAKLKTQTNASDFPIGTSLKYECRPEYYGRPFSITCLDNLVWSSPKDVCKRKSCKTPPDPVNGMVHVITDIQVGSRINYSCTTGHRLIGHSSAECILSGNTAHWSTKPPICQRIPCGLPPTIANGDFISTNRENFHYGSVVTYRCNLGSRGRKVFELVGEPSIYCTSNDDQVGIWSGPAPQCII

SEQ ID NO: 149 - protein, 235 aa, Homo sapiens
IVGGKRAQLGDLPWQVAIKDASGITCGGIYIGGCWILTAAHCLRASKTHRYQIWTTVVDWIHPDLKRIVIEYVDRIIFHENYNAGTYQNDIALIEMKKDGNKKDCELPRSIPACVPWSPYLFQPNDTCIVSGWGREKDNERVFSLQWGEVKLISNCSKFYGNRFYEKEMECAGTYDGSIDACKGDSGGPLVCMDANNVTYVWGVVSWGENCGKPEFPGVYTKVANYFDWISYHVG

SEQ ID NO: 150 - protein, 246 aa, Homo sapiens
EDCNELPPRRNTEILTGSWSDQTYPEGTQAIYKCRPGYRSLGNVIMVCRKGEWVALNPLRKCQKRPCGHPGDTPFGTFTLTGGNVFEYGVKAVYTCNEGYQLLGEINYRECDTDGWTNDIPICEVVKCLPVTAPENGKIVSSAMEPDREYHFGQAVRFVCNSGYKIEGDEEMHCSDDGFWSKEKPKCVEISCKSPDVINGSPISQKIIYKENERFQYKCNMGYEYSERGDAVCTESGWRPLPSCEE

SEQ ID NO: 151 - protein, 194 aa, Homo sapiens; identical to SEQ ID NO: 146

SEQ ID NO: 152 - protein, 194 aa, Homo sapiens; identical to SEQ ID NO: 147

SEQ ID NO: 153 - protein, 342 aa, Homo sapiens
MKGKFLKVSSLFVATLTTATLVSSPAANALSSKAMDNHPQQTQSSKQQTPKIKKGGNLKPLEQREHANVILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 154 - protein, 274 aa, Homo sapiens
VILPNNDRHQITDTTNGHYAPVTYIQVEAPTGTFIASGVVVGKDTLLTNKHVVDATHGDPHALKAFPSAINQDNYPNGGFTAEQITKYSGEGDLAIVKFSPNEQNKHIGEVVKPATMSNNAETQVNQNITVTGYPGDKPVATMWESKGKITYLKGEAMQYDLSTTGGNSGSPVFNEKNEVIGIHWGGVPNEFNGAVFINENVRNFLKQNIEDIHFANDDQPNNPDNPDNPNNPDNPNNPDNPNNPDEPNNPDNPNNPDNPDNGDNNNSDNPDAA

SEQ ID NO: 155 - protein, 12 aa, Homo sapiens
VGCAGFASVTQE

SEQ ID NO: 156 - protein, 14 aa, Homo sapiens
LRRQHARASHLGLA

SEQ ID NO: 157 - protein, 5 aa, Homo sapiens
SASLL

(19) United States
(12) Patent Application Publication
(10) Pub. No.: US 2023/0400470 A1
(43) Pub. Date: Dec. 14, 2023

(54) DETECTION OF COMPLEMENT PROTEINS

(71) Applicant: Complement Therapeutics Limited, London (GB)

(72) Inventors: Paul BISHOP, Stockport (GB); Simon CLARK, Baden-Wurttemberg (DE); Richard UNWIN, Manchester (GB)

(21) Appl. No.: 18/052,795

(22) Filed: Nov. 4, 2022

Related U.S. Application Data
(63) Continuation of application No. PCT/EP2021/062077, filed on May 6, 2021.

(30) Foreign Application Priority Data
May 7, 2020 (GB) 2006789.8

(51) Int. Cl. G01N 33/68

(52) U.S. Cl. CPC G01N 33/6848; G01N 2333/4716; G01N 2333/96433

(57) ABSTRACT

Methods for detecting and determining the level of complement proteins are disclosed, in particular using mass spectrometry. Also disclosed are methods of identifying subjects having or at risk of developing a complement-related disorder, and methods of treating the same.

This application claims priority from GB 2006789.8 filed on 7 May 2020, the contents and elements of which are herein incorporated by reference for all purposes.

### FIELD OF THE INVENTION

The present invention relates to the fields of molecular biology and medicine. More specifically, the present invention relates to methods for detecting complement proteins and the use of such methods for diagnostic and therapeutic uses.

### BACKGROUND TO THE INVENTION

The complement system contributes to innate host immune defence by assisting in the rapid recognition and elimination of microbial intruders. However, dysregulation of complement can contribute to inflammatory, immune-related, and age-related conditions. As a result inappropriate regulation of the complement system has been implicated in a wide variety of diseases in humans e.g. diseases of the eye and kidney, as well as neurological diseases and cancer (Morgan, B. P., Semin Immunopathol, 2018. 40(1): p. 113-124; Halbgebauer, R., et al., Semin Immunol, 2018. 37: p. 12-20; Ma, Y., et al., Aging Dis, 2019. 10(2): p. 429-462; and Kleczko, E. K., et al., Front Immunol, 2019. 10: p. 954.

Complement pathway activation and control is regulated by a complex interplay between pathway activators and inhibitors. These activators and inhibitors are commonly enzymes which cleave and inactivate complement molecules on biological surfaces and/or in solution to maintain steady regulation of complement activating species. The complement pathways are in a constant state of flux and balance, and disturbances to this balance can lead to inappropriate activation and the consequences above.

One activating molecule is complement component 3 (C3), a member of the alternative complement pathway and amplification loop. C3 comprises a β chain and an α′ chain which associate through interchain disulphide bonds. During complement activation, C3 is cleaved to generate two functional fragments, C3a and C3b. C3a is a potent anaphylatoxin. Deposition of C3b on biological surfaces, e.g. extracellular matrix and cell surfaces, is the central activating mechanism of the alternative pathway. C3b is a potent opsonin, targeting pathogens, antibody-antigen immune complexes and apoptotic cells for phagocytosis by phagocytes and NK cells. Surface-linked C3b also reacts with other complement proteins to form active convertase enzymes that are able to produce further (surface-attachable) C3b molecules, serving to activate and amplify complement responses (Clark, S. J., et al., J Immunol, 2014. 193(10): p. 4962-70). C3b associates with Factor B to form the C3bBb-type C3 convertase and with C3bBb to form the C3bBb3b-type C5 convertase. Proteolytic cleavage of C3 also produces C3a and C3b through the classical complement pathway and the lectin pathway.

Insufficient control of C3 convertases results in massive production of C3b and C3a molecules and a shift of the complement cascade to its terminal lytic pathway. This produces the potent anaphylatoxin, C5a, and the cell lytic protein complex termed the membrane attack complex; both providing strong inflammatory signals (Clark, S. J., et al., supra). This ultimately leads to cell/tissue destruction and a local inflammatory response.

C3b activation of complement is regulated by complement protein factor I (FI). FI prevents complement activation by cleaving C3b to a proteolytically-inactive form, designated iC3b, which is unable to participate in convertase assembly, and further to downstream products iC3dg and C3d. FI requires the presence of a cofactor, examples of which include the blood-borne Factor H (FH) protein and the membrane-bound surface co-factor ‘complement factor 1’ (CR1; CD35). FH and CR1 also help to exert decay-accelerating activity, which can assist in the deconstruction of already formed C3 convertases.

FH is encoded by the CFH gene on human chromosome 1q32 within the RCA (regulators of complement) gene cluster. There is a naturally-occurring truncated form of FH called FH-like protein 1 (FHL-1) which arises from alternative splicing of the CFH gene and has cofactor activity like FH. FH comprises 20 CCP domains. FHL-1 is identical to FH for the first seven CCP domains before terminating with a unique 4-aa C terminus.

Proteins encoded by the CFHR1-5 genes at the RCA locus also exert complement regulatory functions. The CFHR1-5 genes encode a group of five secreted plasma proteins (FHR-1 to FHR-5) synthesised primarily by hepatocytes. The FHR proteins retain some sequence homology with C3b binding domains of FH and are thought to enhance complement activation (Skerka et al., Mol Immunol. 2013, 56:170-180).

One complement-related disorder is macular degeneration, e.g. age-related macular degeneration (AMD). Macular degeneration is believed to be driven in part by complement-mediated attack on ocular tissues. A major driver of AMD risk is genetic variation at the RCA locus resulting in dysregulation of the complement cascade. AMD is the leading cause of blindness in the developed world: currently responsible for 8.7% of all global blind registrations. It is estimated that 196 million people will be affected by 2020, increasing to 288 million by 2040 (Wong et al. Lancet Glob Heal (2014) 2:e106-16). AMD manifests as the progressive destruction of the macula, the central part of the retina at the back of the eye, leading to loss of central visual acuity. Early stages of the disease see morphological changes in the macula such as the loss of blood vessels in the choriocapillaris (Whitmore et al., Prog Retin Eye Res (2015) 45:1-29); a layer of capillaries found in the choroid (a highly vascularized layer that supplies oxygen and nutrition to the outer retina). The choriocapillaris is separated from the metabolically active retinal pigment epithelium (RPE) by Bruch's membrane (BrM); a thin (2-4 μm), acellular, five-layered sheet of extracellular matrix. The BrM serves two major functions: the substratum of the RPE and a blood vessel wall. The structure and function of BrM is reviewed e.g. in Curcio and Johnson, Structure, Function and Pathology of Bruch's Membrane, In: Ryan et al. (2013), Retina, Vol. 1, Part 2: Basic Science and Translation to Therapy. 5th ed. London: Elsevier, pp466-481, which is hereby incorporated by reference in its entirety.

The role of complement in AMD is reviewed, for example, by Zipfel et al. Chapter 2, in Lambris and Adamis (eds.), Inflammation and Retinal Disease: Complement Biology and Pathology, Advances in Experimental Medicine and Biology 703, Springer Science+Business Media, LLC (2010), which is hereby incorporated by reference in its entirety. The key characteristics of AMD are indicative of over-active complement, including cell/tissue destruction and a local inflammatory response. Hallmark lesions of early AMD, termed drusen, develop within BrM adjacent to the RPE layer (Bird et al, Surv Ophthalmol 1995, 39(5):367-374). Drusen are formed from the accumulation of lipids, proteins and cellular debris, and include a swathe of complement activation products (Anderson et al., Prog Retin Eye Res 2009, 29:95-112; Whitcup et al., Int J Inflam 2013, 1-10). The presence of drusen within BrM disrupts the flow of nutrients from the choroid across this extracellular matrix to the RPE cells, which leads to cell dysfunction and eventual death, leading to the loss of visual acuity.

‘Dry’ AMD, also known as geographic atrophy, represents around 50% of late-stage AMD cases. In the remaining percentage of late-stage cases, choroidal neovascularisation (CNV) develops, in which the increased synthesis of vascular endothelial growth factor (VEGF) by RPE cells promotes new blood vessel growth from the choroid/choriocapillaris that breaks through BrM into the retina. These new blood vessels leak and eventually form scar tissue; this is referred to as ‘wet’ (neovascular or exudative) AMD. ‘Wet’ AMD is the most virulent form of late-stage AMD and has different disease characteristics to ‘dry’ AMD. There are treatments for wet AMD, where for example the injection of anti-VEGF agents into the vitreous of the eye can slow or reverse the growth of these blood vessels, although it cannot prevent their formation in the first place. Geographic atrophy (‘dry’ AMD) remains untreatable.

FHL-1 predominates at BrM, suggesting an important role for this variant in protection of retinal tissue from complement-mediated attack (Clark, S. J., et al., supra). FH is found in the blood at a higher concentration than FHL-1. Both FH and FHL-1 protect against complement over-activation in the ECM of the choroid (the capillary network underlying BrM). The role of the five FHR proteins are less well understood, although there is some evidence that they may counter the inhibitory effects of FH and FHL-1 (Clark, S. J. and P. N. Bishop, J Clin Med, 2015. 4(1): p. 18-31).

WO2019/215330 describes that FHR-4 is a positive regulator of complement activation and prevents FH-mediated C3b breakdown, leading to the formation of C3 convertase and the progression of the complement activation loop. High levels of circulating FHR-4 indicate an increased risk of developing complement-related disorders.

Defining the exact molecular changes and activation state underpinning the dysregulation of complement processes in human disease tissue remains problematic, largely because it requires measurements at the protein level and an understanding of the relative quantities of the different regulators. It is critical to be able to accurately measure absolute levels of FH and related RCA locus proteins in plasma, as well as levels of FI and C3b itself, for effective diagnosis and treatment of complement-related disorders. Whilst assays have been developed for FH, distinct measurement of FHL-1 and FHR1-5 is difficult due to high sequence homology between all these proteins. This sequence similarity has meant that, with the exception of the full length FH protein, it has proven difficult to generate antibodies which are specific to only one of these family members in order to obtain useful immunoassays.

In another example, a recent study quantified levels of FH and FHR1-5 using mass spectrometry but the assay was unable to detect biologically important isoform FHL-1 that is found at significant levels in the blood and at key sites of AMD pathogenesis (Zhang, P., et al., Proteomics. 2017;17(6):10).

The present invention seeks to address these issues.

### SUMMARY OF THE INVENTION

The present invention relates to detecting complement proteins using mass spectrometry. The present application describes methods that are capable of distinguishing between different Complement Factor H, FHL-1 and the five Complement Factor H related (FHR) proteins, despite their sequence similarity. The method can also distinguish between breakdown products derived from C3, and other complement-related proteins with high sequence similarity.

Provided is a method for detecting at least one complement protein in a sample, the method comprising digesting the protein(s) with endoproteinase GluC to obtain one or more peptides; and detecting the one or more peptides by mass spectrometry.

The invention also provides a method for determining the level of at least one complement protein in a sample, the method comprising digesting the protein(s) with endoproteinase GluC to obtain one or more peptides; and determining the level of the one or more peptides by mass spectrometry.

In some embodiments the step of detecting the one or more peptides, and/or determining the level of the one or more peptides, consists of measuring the one or more peptides by mass spectrometry. In some embodiments the step of detecting the one or more peptides, and/or determining the level of the one or more peptides, involves measuring the level of the one or more peptides by mass spectrometry alone.

In some embodiments the method comprises determining the concentration of the complement protein(s) in the sample.

In another aspect the invention provides a method for preparing at least one complement protein for analysis, the method comprising digesting the protein(s) with endoproteinase GluC to obtain one or more peptides.

In some embodiments the complement protein(s) is one or more of FH, FHL-1, FHR1, FHR2, FHR3, FHR4 and/or FHR5. In some embodiments the complement protein(s) is FH and/or FHL-1. In some embodiments the complement protein(s) is involved in the complement amplification loop and/or C3 convertase activity. In some embodiments the complement protein(s) is a breakdown product of C3b. In some embodiments the complement protein(s) is one or more of C3, C3b, C3a, iC3b, C3f, C3c, C3dg, and/or C3d. In some embodiments the complement protein(s) is one or more of C3, C3a, C3f, C3c, and/or C3d. In some embodiments the complement protein(s) is C3b and/or iC3b. In some embodiments the complement protein is FI. All combinations of the above proteins are envisaged. The methods described herein may involve detecting/determining the level of two or more complement proteins. In some embodiments, the method comprises simultaneous detection/determination of the levels of the two or more complement proteins.

In some embodiments the sample has been obtained from a subject. In some embodiments the method comprises a step of obtaining the sample from a subject. In some embodiments the sample comprises, or is derived from, blood, lymph, plasma, serum, tissue or cells.

The peptides may be any suitable peptide disclosed herein. The one or more peptides may be selected from the group consisting of:

(a)
(SEQ ID NO: 20)
VTYKCFE;
(b)
(SEQ ID NO: 21)
NGWSPTPRCIRVSFTL;
(c)
(SEQ ID NO: 22)
ATFCDFPKINHGILYDEE;
(d)
(SEQ ID NO: 23)
RGWSTPPKCRSTISAE
or
(SEQ ID NO: 24)
AMFCDFPKINHGILYDEE;
(e)
(SEQ ID NO: 25)
VACHPGYGLPKAQTTVTCTE;
(f)
(SEQ ID NO: 26)
YQCQSYYE;
(g)
(SEQ ID NO: 27)
RGWSTPPICSFTKGE;
(h)
(SEQ ID NO: 28)
GTAFVIFGIQDGE;
(i)
(SEQ ID NO: 29)
LRRQHARASHLGLARSNLDE;
(j)
(SEQ ID NO: 30)
LRRQHARASHLGLAR
and/or
(SEQ ID NO: 156)
LRRQHARASHLGLA;
(k)
(SEQ ID NO: 31)
LNLDVSLQLPSRSSKITHRIHWE;
(l)
(SEQ ID NO: 32)
LNLDVSLQLPSR;
(m)
(SEQ ID NO: 33)
RLGRE;
(n)
(SEQ ID NO: 34)
SSKITHRIHWE;
(o)
(SEQ ID NO: 35)
SASLLR
and/or
(SEQ ID NO: 157)
SASLL;
(p)
(SEQ ID NO: 36)
RLGR;
(q)
(SEQ ID NO: 37)
HLIVTPSGCGE;
and/or
(r)
any one or more of SEQ ID NO: 38 to 60.

The invention also provides the use of endoproteinase GluC for preparing at least one complement protein for detection by mass spectrometry, optionally for preparing at least two complement proteins for detection by mass spectrometry, e.g. any combination of proteins described herein.

Also provided is a method for detecting turnover of C3b, comprising a method for detecting at least one complement protein in a sample e.g. as described herein.

Also provided is a method of determining the presence and/or level of a complement protein in a subject, the method comprising performing a method as described herein. In some embodiments the method is performed on a sample obtained from the subject.

The invention also comprises a method of determining whether a subject is at risk of developing a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine the likelihood of the subject to develop a complement-related disorder.

Also provided is a method of identifying a subject having a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject has a complement-related disorder.

In some embodiments the methods comprise (d) treating a subject who has been determined to be at risk of developing, or to have, a complement-related disorder. Treating a subject may comprise administering a therapeutically effective amount of a complement-targeted therapeutic to the subject.

Also provided is a method of selecting a subject for treatment of a complement-related disorder with a complement-targeted therapeutic, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject is in need of a complement-targeted therapeutic.

Also provided is a method of treating a subject who is suspected to have a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and based on the results of (b), treating said disorder.

In some embodiments the methods comprise administering an effective amount of a complement-targeted therapeutic to the subject, e.g. a subject identified as having a disorder or selected for treatment as described herein.

Also provided is a complement-targeted therapeutic for use in a method of treating a complement-related disorder in a subject, the method comprising:
a) digesting at least one complement protein in a sample obtained from the subject with endoproteinase GluC to obtain one or more peptides;
b) determining the presence and/or level of the one or more peptides by mass spectrometry; and
c) based on the results of (b), administering an effective amount of the complement-targeted therapeutic.

In some embodiments the methods comprise obtaining a sample from a subject comprising at least one complement protein. The sample may comprise or be derived from blood, lymph, plasma, serum, tissue or cells.

In some embodiments the complement-related disorder is macular degeneration. In some embodiments the complement-related disorder is selected from AMD, geographic atrophy (‘dry’ (i.e. non-exudative) AMD), early AMD, EOMD, intermediate AMD, late/advanced AMD, ‘wet’ (neovascular or exudative) AMD, choroidal neovascularisation (CNV) and/or retinal dystrophy.

In some embodiments the complement-related disorder is selected from Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, neurodegeneration/neurodegenerative disease, dementia, multiple sclerosis (MS), cancer, stroke, Parkinson's disease, and/or Alzheimer's disease.

In any of the diagnostic or therapeutic methods described herein the complement protein may be any one or more of the proteins provided herein. i.e. any one or more, or any combination of, FH, FHL-1, FHR1, FHR2, FHR3, FHR4, FHR5, FI, C3, C3b, C3a, iC3b, C3f, C3c, C3dg, and/or C3d. The protein(s) may be involved in the complement amplification loop and/or C3 convertase activity. The complement protein may be a breakdown product of C3b. In some embodiments the complement protein(s) is one or more of FH, FHL-1, FHR1, FHR2, FHR3, FHR4 and/or FHR5. In some embodiments the complement protein is FH and/or FHL-1. In some embodiments the complement protein(s) is one or more of C3, C3b, C3a, iC3b, C3f, C3c, C3dg, and/or C3d. In some embodiments the complement protein(s) is one or more of C3, C3a, C3f, C3c, and/or C3d. In some embodiments the complement protein(s) is C3b and/or iC3b. In some embodiments the complement protein is FI. The one or more peptides may be any suitable peptide such as those disclosed herein, e.g. any one of SEQ ID NO:20 to 60.

In some embodiments an increase in the level of one or more of C3, C3b, C3a, iC3b, FHR1, FHR2, FHR3, FHR4 and/or FHR5; and/or a decrease in the level of one or more of iC3b, C3f, C3c, C3dg, C3d, C3g, FI, FH, FHL-1, FHR1, FHR2, FHR3, FHR4 and/or FHR5 as compared to reference value(s), indicates that the subject is at risk of developing or has a complement-related disorder.

Also provided is a kit for use in a method of detecting and/or determining the level of one or more complement protein(s) in a sample, the kit comprising endoproteinase GluC.

### DESCRIPTION

Detection, differentiation and quantitation of highly similar proteins can be achieved using mass spectrometry (MS). In order to achieve good sensitivity by MS, proteins e.g. in a sample are routinely digested into peptides using a specific protease. The industry standard protease for this purpose is trypsin. Other enzymes that are commonly used to digest proteins for MS analysis include elastase, chymotrypsin or LysN.

Trypsin cleaves C-terminal to all K and R residues, provided they are not followed by a proline residue, and yields peptides which retain a basic group at their C-terminus which subsequently helps ionisation and transmission of peptides into the gas phase in a mass spectrometer. Peptides digested by trypsin tend to be ionised more efficiently during MS and thus produce a larger signal than peptides digested by non-trypsin enzymes. Using MS, individual peptides in the sample digest can be detected with a signal proportional to its abundance. The concentration of the parent protein can be derived from the relative abundance (signal) of endogenous peptide compared to an exogenous ‘standard’ peptide e.g. containing a stable isotope.

However, trypsin digestion of complement proteins FH and FHL-1 does not produce peptides that can be detected individually using MS alone. The only FHL-1 specific tryptic peptide is a 4-amino acid C-terminal sequence which is too small to be detected reliably by MS techniques. The FHR proteins also share substantial sequence identity, meaning that it is hard to distinguish between them and measure them specifically using e.g. antibody-based assays.

The inventors have developed a unique targeted mass spectrometry assay using a non-standard proteolytic enzyme, GluC (V8 protease), to produce distinct proteotypic peptides for all of the FHR proteins, as well as proteotypic peptides that can be used to distinguish between FHL-1 and FH, which can be used for the simultaneous detection and accurate measurement in plasma of all seven key regulatory proteins encoded from the CFH gene cluster using a single MS assay, i.e. simultaneous detection of FH, FHL-1, and FHR1, FHR2, FHR3, FHR4 and FHR5.

FHL-1 is a distinct biological entity from FH. The proteins have a similar action but the size of FHL-1 means that its distribution in the body is likely to be distinct from FH. This is apparent in the eye where FHL-1 can cross to the retinal side of Bruch's membrane, e.g. where drusen form, but the larger FH protein cannot, see e.g. Clark et al., J Immunol 2014, 193(10) 4962-4970 and and Clark et al., Frontiers in Immunology 2017 8:177, which are hereby incorporated by reference in their entirety. In this respect, there is evidence that FHL-1 is the prime driver of complement C3b turnover in the eye, meaning that levels of FHL-1 are likely to better inform disease risk than levels of FH.

GluC is also able to produce proteotypic peptides for C3b and FI, enabling direct measurement of C3b itself as well as levels of its proteolytic enzyme and required fluid-phase cofactors. Thus, the methods described herein mean that all these complement proteins can be measured using a single assay. Furthermore, breakdown of C3b occurs via trypsin-like cleavages at basic residues (K and R) so trypsin digestion of C3b breakdown products is unable to produce useful peptides for analysis. In contrast, C3 turnover can be measured using the MS approach of the present invention because GluC digestion also produces proteotypic neopeptides from many C3 inactivation and breakdown products generated during inactivating cleavages. The inventors demonstrate herein that a series of products produced as a result of C3/C3b cleavage can be detected and quantified using the same single GluC/MS assay. This allows the concentrations of all known C3 fragments e.g. iC3b, C3c, C3dg and C3d to be determined accurately. Thus, the methods described herein can not only measure absolute levels of regulatory complement proteins, but can also track protein products resulting from C3 inactivation and thus assess complement activation and the progression of the amplification loop.

This is advantageous because the measurement of C3 breakdown products is analytically challenging. The pattern by which C3 is broken down is complex: first into C3a and C3b, followed by cleavage of C3b into iC3b (which cannot drive formation of the membrane attack complex (MAC) but can still act as an opsonin), and then subsequent inactivating cleavage of iC3b into C3c via the release of a C3dg fragment. To approach detection of these products with antibodies is problematic. While each sequential cleavage step in this cascade generates a new proteoform (a distinct form of a protein encoded from the same gene, including cleaved forms and splice variants), they share sequence homology and likely only undergo minor structural changes. Directing antibodies at each form is likely to be unsuccessful and while there are methods which can measure single components following some form of separation, e.g. C3dg following polyethylene glycol-based enrichment, simultaneous measurement of all fragments in the same sample is not currently possible.

Thus, the present invention relates to a single methodology for concurrent determination of the presence, absolute levels and relative molar ratios of seven individual complement-related proteins from the CFH family plus C3b-inactivating enzyme FI, central complement component C3, and seven proteins derived from C3 breakdown, which may be referred to herein as the “complementome”. The ability to detect absolute levels of so many complement-related proteins in one assay is critical for the successful detection, diagnosis and treatment of complement-related diseases.

### Complement Proteins

Complement is a central part of the innate immunity that serves as a first line of defence against foreign and altered host cells. Complement is activated upon infection with microorganisms to induce inflammation and promote elimination of the pathogens. The complement system is composed of plasma proteins produced mainly by the liver or membrane proteins expressed on cell surface. Complement operates in plasma, in tissues, or within cells. For a review of the complement system, see e.g. Merle N S et al., Front Immunol. 2015 Jun :2;6:262, which is hereby incorporated by reference in its entirety.

The complement system can be activated via three distinct pathways: the classical pathway (CP), alternative pathway (AP) and lectin binding pathway (LP). In a healthy individual, the AP is permanently active at low levels to survey for presence of pathogens but host cells are protected against complement attack and are resistant to persistent low-level activation. C3b molecules bound to host cells are inactivated rapidly by a group of membrane-bound or plasma complement regulators.

In response to the recognition of molecular components of microorganisms, complement proteins become sequentially activated in an enzyme cascade: the activation of one protein enzymatically cleaves and activates the next protein in the cascade.

The three pathways converge into the generation of a C3 convertase, which cleaves the central complement component C3 into activation products C3b, a large fragment that acts as an opsonin (binds to foreign microorganisms to increase their susceptibility to phagocytosis), and C3a, an anaphylatoxin that promotes inflammation. Along with factor B (FB), C3b forms the C3 convertase (C3bBb) which cleaves further C3 molecules, generates more C3b and C3a, and amplifies C3b deposition on cell surfaces. This is the complement amplification loop. C3b deposition and activation of complement may occur on acellular structures (i.e. on extracellular matrix), such as Bruch's membrane (BrM) and the intercapillary septa of the choriocapillaris in the eye.

Activated C3 can trigger the lytic pathway, which can damage the plasma membranes of cells and some bacteria. C5a, another anaphylatoxin produced by this process, attracts macrophages and neutrophils and also activates mast cells.

Once activated, the complement system needs tight control, as newly generated complement activation products, e.g. C3b, can induce severe inflammation and cell damage to the host. A number of soluble as well as membrane bound complement regulators ensure regulation of complement activation at the surface of host cells and control different activation phases and sites of action (Skerka et al., Mol Immunol 2013, 56:170-180). Complement regulators are described further herein.

“Complement protein” may be used interchangeably herein with “complement regulator”, “a regulator of complement”, or “protein of the complement system” and refers to a protein component of the complement system or complement cascade, e.g. as described in Merle et al., Front. Immunol., 2015, 6:262 and Merle et al., Front. Immunol., 2015, 6:257, which are hereby incorporated by reference in their entirety. A “complement protein” referred to herein may be involved in any of the three complement pathways and/or in the amplification loop.

In some embodiments a “complement protein” referred to herein is involved in the alternative pathway and/or the complement activation loop. In some embodiments, a “complement protein” referred to herein is involved in the breakdown, turnover and/or inactivation of C3 or C3b, or is a product of said breakdown, turnover and/or inactivation.

In some embodiments, a “complement protein” as used herein may refer to one or more of FH, FHL-1, FHR1, FHR2, FHR3, FHR4, FHRS, FI, C3, C3b, C3a, iC3b, C3f, C3c, C3dg, and/or C3d.

### Complement Factor H (FH) Protein Family

Factor H (FH) regulates the alternative complement pathway and the amplification loop. It inhibits C3 convertase formation by competing with FB binding to C3b and also acts as a cofactor for C3b inactivation to iC3b by Factor I (FI), thus preventing inappropriate complement activation and inflammation. FH also exerts decay-accelerating activity, which can assist in the deconstruction of already formed C3 convertases, see e.g. Clark et al., J Immunol 2014, 193(10) 4962-4970, which is hereby incorporated by reference in its entirety.

The sequence of human FH (Uniprot P08603-1) is provided herein as SEQ ID NO:1. For a review of FH structure and function see e.g. Merle N S et al., Front Immunol. 2015 Jun 2;6:262, which is hereby incorporated by reference in its entirety.

Human FH comprises 20 CCP domains. The CFH gene also produces a truncated form of FH, called FHL-1, comprising only the first seven CCP domains before terminating with a unique 4-amino acid C terminus (Clark et al, 2014 supra). The sequence of human FHL-1 (Uniprot: P08603-2) is provided herein as SEQ ID NO:2.

In the eye, full-length FH protein is found on the choroidal side of Bruch's membrane (BrM), with particular accumulation in the choriocapillaris (capillary layer in the choroid). Small amounts have also been found in patches on the RPE side of the BrM, but no FH was observed in the BrM itself. FHL-1 on the other hand has been observed throughout BrM and other ECM structures e.g. drusen (Clark et al, 2014 supra). It is likely that FHL-1 confers greater complement protection to BrM than does FH, whereas FH provides the main protection for the ECM of the choroid. It is thought that FHL-1 is therefore a major regulator of complement in the BrM (a key site in AMD pathogenesis). The methods described herein allow for the individual detection and quantitation of FH and FHL-1.

The CFHR1-5 genes encode a group of five secreted plasma proteins (FHR-1 to FHR-5) synthesised primarily by hepatocytes. FH, FHL-1 and FHR1-FEIR5 are described in e.g. Clark et al., J Clin Med, 2015. 4(1): 18-31, which is hereby incorporated by reference in its entirety.

The FHR proteins retain some sequence homology with C3b binding domains of FH and are thought to enhance complement activation, see e.g. Skerka et al., Mol Immunol 2013, 56:170-180, which is hereby incorporated by reference in its entirety. These proteins are highly related and share a high degree of sequence identity. The N termini share 36-94% sequence identity, whilst the C-terminal domains are very similar to the FH C-terminus (36-100%). The high amino acid identity among family members is demonstrated by the fact that antibodies raised against FH can detect multiple FHR proteins in plasma and that antibodies generated against FHR proteins cross-react with the other FHRs. This cross-reactivity presents a challenge for purification of FHR proteins from plasma, as well as determining their concentration.

FHR proteins are divided into two groups depending on their conserved domains. FHR1 (SEQ ID NO:3), FHR2 (SEQ ID NO:3, 4), and FHR5 (SEQ ID NO:10) form Group I and are characterised by their conserved N-termini. They exist in plasma as homo- and heterodimers, mediated by the conserved N-terminal domains. Group II contains FHR3 (SEQ ID NO:6, 7) and FHR4 (SEQ ID NO:8, 9) which lack the N-terminal dimerisation domains, but which show a high degree of sequence similarity to portions of FH. All five FHR proteins comprise C-termini sequences that act to recognise and bind C3b, and which are very similar to the C-terminus of FH.

FHR1 is known to compete with FH and FHL-1 for binding to C3b. It is also reported to bind to C3b components of the C5 convertase and interfere with the assembly of the MAC (see e.g. Heinen S et al., Blood (2009) 114 (12): 2439-2447 and Hannan J P et al., PLoS One. 2016; 11(11):e0166200, which are hereby incorporated by reference in their entirety). As used herein, the term “FHR1” includes at least one of FHR1 (SEQ ID NO:3; FHRA) and a second FHR1 isoform (FHRB) with 3 point mutations, and preferably includes both FHR1 isoforms. “FHR1” refers to FHR1 from any species and includes isoforms, fragments, variants or homologues of FHR1 from any species. In preferred embodiments, “FHR1” refers to human FHR1.

FHR2 may inhibit C3 convertase activity, acting to inhibit the amplification loop, but may also activate the amplification loop. There are two FHR2 isoforms (SEQ ID NO:4 and 5). The protein has two glycosylated forms, a single glycosylated form (24 kDa) and a double glycosylated form (28 kDa). As used herein, the term “FHR2” includes at least one of the two isoforms or at least one of the glycosylated forms, and preferably includes both isoforms and any glycosylated forms. “FHR2” refers to FHR2 from any species and includes isoforms, fragments, variants or homologues of FHR2 from any species. In preferred embodiments, “FHR2” refers to human FHR2.

FHR3 binds to C3b and C3d and may have low cofactor activity for FI-mediated cleavage of C3b. FHR3 may also upregulate complement. There are two FHR3 isoforms (SEQ ID NO:6 and 7). FHR3 is detected in plasma in multiple variants (ranging from 35 to 56 kDa), reflecting the existence of four different glycosylated variants of FHR3. As used herein, the term “FHR3” includes at least one of the two isoforms or at least one of the glycosylated variants of FHR3, and preferably includes both isoforms and any glycosylated forms. “FHR3” refers to FHR3 from any species and includes isoforms, fragments, variants or homologues of FHR3 from any species. In preferred embodiments, “FHR3” refers to human FHR3.

The human CFHR4 gene encodes two proteins: FHR4A (SEQ ID NO:8) and FHR4B (SEQ ID NO:9), an alternative splice variant. WO 2019/215330, hereby incorporated by reference in its entirety, describes that FHR4 is a positive regulator of complement activation and prevents FH-mediated C3b breakdown. High levels of FHR-4 in tissues are likely to promote local inflammatory responses and cell lysis, leading to disorders associated with complement activation, and circulating FHR4 levels can be used as an indicator of risk of developing complement-related disorders, see e.g. Cipriani et al., Nat Commun 11, 778 (2020), hereby incorporated by reference in its entirety. As used herein, the term “FHR4” includes at least one of FHR4A isoform 1, FHR4A isoform 2 (G20 point deletion from isoform 1) or FHR4B, and preferably includes FHR4A isoforms 1 and 2 as well as FHR4B. “FHR4” refers to FHR4 from any species and includes isoforms, fragments, variants or homologues of FHR4 from any species. In preferred embodiments, “FHR4” refers to human FHR4.

FHR5 also recognises and binds to C3b on self surfaces. FHR5 appears as a glycosylated protein of 62 kDa. As used herein, the term “FHR5” includes any glycosylated variants of FHR5, and preferably includes all isoforms and any glycosylated forms. As used herein, “FHR5” refers to FHR5 from any species and includes isoforms, fragments, variants or homologues of FHR5 from any species. In preferred embodiments, “FHR5” refers to human FHR5.

Given the different roles of the different members of the CFH family in activation and amplification of complement and pathogenesis of complement-related disorders, it is important to be able to distinguish between the presence and levels of all seven CFH family members. CFH family members, particularly FHR1-5, can also be used as biomarkers for diagnosing or predicting disorders in which dysregulation of complement is pathologically implicated.

### C3, C3b and Breakdown Products

C3 is the central complement component. The pathways by which C3 is processed into various downstream products can lead to activation of complement, e.g. including inflammation and immune responses, or to the inactivation and regulation of complement. It is therefore important in terms of complement pathogenesis and treatment of complement-related disorders to be able to detect and measure the levels, including relative levels, of C3, C3b and their downstream components/processing products.

Processing of C3 is described, for example, in Foley et al. J Thromb Haemostasis (2015) 13: 610-618, which is hereby incorporated by reference in its entirety. Human C3 (UniProt: P01024; SEQ ID NO:12) comprises a 1,663 amino acid sequence (including an N-terminal, 22 amino acid signal peptide). Amino acids 23 to 667 encode C3 β chain (SEQ ID NO:13), and amino acids 749 to 1,663 encode C3b α′ chain (SEQ ID NO:14). C3 β chain and C3 α′ chain associate through interchain disulphide bonds (formed between cysteine 559 of C3 β chain, and cysteine 816 of the C3 α′ chain) to form C3b. C3a is a 77 amino acid fragment corresponding to amino acid positions 672 to 748 of C3 (SEQ ID NO:15), generated by proteolytic cleavage of C3 to form C3b.

Processing of C3b to the inactive form iC3b, which cannot itself promote further complement amplification, involves proteolytic cleavage of the C3b α′ chain at amino acid positions 1303 and 1320 to form an α′ chain fragment 1 (corresponding to amino acid positions 749-1663 of C3; SEQ ID NO:16), and an α′ chain fragment 2 (corresponding to amino acid positions 1321 to 1,663 of C3; SEQ ID NO:17). Thus, iC3b comprises the C3 β chain, C3 α′ chain fragment 1 and C3 α′ chain fragment 2 (associated via disulphide bonds). Cleavage of the α′ chain also liberates C3f, which corresponds to amino acid positions 1304 to 1320 of C3 (SEQ ID NO:18).

iC3b is processed further to C3c comprising the C3 β chain, C3 α′ chain fragment 2 and C3c α′ chain fragment 1 (corresponding to amino acid positions 749-954 of C3; SEQ ID NO:19). This cleavage event produces fragment C3dg (corresponding to amino acid positions 955-1303 of C3; SEQ ID NO:142), which is itself broken down into fragments C3g (corresponding to amino acid positions 955-1001 of C3; SEQ ID NO:143) and C3d (corresponding to amino acid positions 1002-1303 of C3; SEQ ID NO:144).

Processing of C3b to iC3b is performed by Complement Factor I (FI; encoded in humans by the gene CFI). Human Complement Factor I (UniProt: P05156; SEQ ID NO:11) has a 583 amino acid sequence (including an N-terminal, 18 amino acid signal peptide). Amino acids 340 to 574 of the light chain encode the proteolytic domain of FI, which is a serine protease containing the catalytic triad responsible for cleaving C3b to produce iC3b (Ekdahl et al., J Immunol (1990) 144 (11): 4269-74). Proteolytic cleavage of C3b by FI to yield iC3b is facilitated by co-factors, including FH, CR1 and possibly some of the FHR proteins. Co-factors for FI typically bind to C3b and/or FI, and potentiate processing of C3b to iC3b by FI.

As used herein, any reference to a complement protein, e.g. C3, C3b, C3a, FH, FI etc, refers to said protein from any species and include isoforms, fragments, variants or homologues of said protein from any species. In some embodiments, the protein is a mammalian protein (e.g. cynomolgous, human and/or rodent (e.g. rat and/or murine) protein). Isoforms, fragments, variants or homologues of the complement proteins described herein may optionally be characterised as having at least 70%, preferably one of 80%, 85%, 90%, 91%, 92%, 93%, 94%, 95%, 96%, 97%, 98%, 99% or 100% amino acid sequence identity to the amino acid sequence of the immature or mature protein from a given species, e.g. human protein sequences provided herein. Isoforms, fragments, variants or homologues of complement proteins described herein may optionally be functional isoforms, fragments, variants or homologues, e.g. having a functional property/activity of the reference protein, as determined by analysis by a suitable assay for the functional property/activity.

### Methods for Detecting Complement Proteins

The present invention relates to detecting the presence of, and/or determining the level of, one or more complement proteins using suitable analytical techniques, e.g. as described herein.

In some aspects the present invention provides a method for detecting a complement protein, the method comprising contacting the protein with endoproteinase GluC to obtain one or more peptides, and detecting the one or more peptides by mass spectrometry.

In some aspects the present invention provides a method for determining the level of a complement protein, the method comprising contacting, e.g. digesting, the protein with GluC to obtain one or more peptides, and determining the level of the one or more peptides by mass spectrometry. In some cases, the methods involves both detecting a complement protein and determining the level of a complement protein. The protein may be the same protein, or the methods may involve detection of a first complement protein and determining the level of a second complement protein.

In any and all methods described herein, the step of detecting/determining the level of the one or more peptides consists of detecting/determining the level of/measuring the peptide(s) by mass spectrometry. That is, the step of detecting/determining the level of/measuring the peptide(s) is performed by mass spectrometry only. Measuring the peptide(s) may include detecting the presence or absence of the one or more peptides, and/or determining the level, amount and/or concentration of each peptide in the sample.

The term “digesting” as used herein refers to placing the protein in contact with GluC under suitable conditions, e.g. temperature, pH etc, and for a suitable time such that the protein is digested, i.e. cleaved, into two or more fragments. In some cases, the digesting involves incubating the protein with GluC under suitable conditions, e.g. as described herein.

In some aspects the present invention provides a method for preparing a complement protein for analysis, the method comprising contacting/digesting the protein with endoproteinase GluC to obtain one or more peptides. In some cases, the method comprises preparing a complement protein for subsequent analysis. The one or more peptides may then be subjected to an analytical technique, e.g. mass spectrometry or any other suitable analytical technique. In some cases the method comprises preparing a complement protein for analysis by mass spectrometry. The analytical technique may be used to detect the presence and/or level of the one or more peptides.

It will be appreciated that where “complement protein” is referred to herein in the singular (i.e. “a/the complement protein”), pluralities/groups/populations of different complement proteins are also contemplated. For example, any disclosure herein comprising a complement protein also comprises more than one complement protein, i.e. at least one protein, or one or more proteins. In all aspects and embodiments described herein, “a/the complement protein” may refer to “at least one complement protein”.

“Detecting” a protein as used herein refers to identifying/observing the presence or existence of the protein, e.g. in a sample, cell, tissue or subject.

The “level” of a complement protein used herein refers to the level, amount or concentration of said protein, e.g. in a sample, cell, tissue or subject. The term “determining the level”, e.g. of a protein, used herein refers to the measurement and/or quantification of the level, amount or concentration of a protein. In some cases, “determining the level” includes calculating the level, amount or concentration of a protein in a sample. The sample may be from a subject. In some cases, “determining the level” includes calculating the level, amount or concentration of a protein in a subject, e.g. using a sample taken from the subject. “Determining the level” of a protein may include digesting the protein with GluC to obtain one or more peptides, detecting the one or more peptides as described herein and then calculating the level, amount or concentration of the protein/peptide, e.g. in a sample.

In some cases, “determining the level” comprises quantifying, i.e. measuring the quantity of, the level, amount or concentration of a protein e.g. in a sample or in a subject. “Determining the level” may include determining the concentration of a protein. Quantification/measuring may include comparing the level, amount or concentration of a protein with a reference value, and/or comparing the level, amount or concentration of a protein with that in a control sample e.g. taken from the subject at a different time point, or taken from a healthy subject, e.g. one known not to have a complement-related disorder.

In methods described herein the level of the complement protein(s) is compared to the level of a reference value or level, sometimes called a control. In some cases the level of the complement protein(s) is compared to the level of the same complement protein in a control subject that does not have a complement-related disorder. A reference value may be obtained from a control sample, which itself may be obtained from a control subject. Data or values obtained from the individual to be tested, e.g. from a sample, can be compared to data or values obtained from the control sample. In some cases, the control is a spouse, partner, or friend of the subject.

As used herein the term “reference value” refers to a known measurement value used for comparison during analysis. In some cases, the reference value is one or a set of test values obtained from an individual or group in a defined state of health. The reference value may be one or a set of test values obtained from a control. In some cases, the reference value is/has been obtained from determining the level of complement proteins in subjects known not to have a complement-related disorder. In some cases, the reference value is set by determining the level or amount of a complement protein previously from the individual to be tested e.g. at an earlier stage of disease progression, or prior to onset of the disease. The reference value may be taken from a sample obtained from the same subject, or a different subject or subject(s). The sample may be derived from the same tissue/cells/bodily fluid as the sample used by the present invention. The reference value may be a standard value, standard curve or standard data set. Values/levels which deviate significantly from reference values may be described as atypical values/levels.

In some cases the control may be a reference sample or reference dataset, or one or more values from said sample or dataset. The reference value may be derived from a reference sample or reference dataset. The reference value may be derived from one or more samples that have previously been obtained from one or more subjects that are known not to have a complement-related disorder and/or known or expected not to be at risk of developing a complement-related disorder. The reference value may be derived from one or more samples that have previously been obtained from one or more subjects that are known to have a complement-related disorder. The reference value may be derived from one or more samples that have previously been obtained from one or more subjects that are known to be at risk of developing a complement-related disorder. The reference value may be an average, or mean, value calculated from a reference dataset, e.g. a mean protein level. The reference dataset/value may be obtained from a large-scale study of subjects known to have a complement-related disorder.

The reference value may be derived from one or more samples that have previously been obtained from one or more subjects that are in the same family as the subject of interest, or from one or more subjects that are not in the same family as the subject of interest.

The reference value may be derived from one or more samples that have previously been obtained and/or analysed from the individual/subject/patient to be tested, e.g. a sample was obtained from the individual when they were at an earlier stage of a complement-related disorder, or a sample was obtained from the individual before the onset of a complement-related disorder.

The reference value may be obtained by performing analysis of the sample taken from a control subject in parallel with a sample from the individual to be tested. Alternatively, the control value may be obtained from a database or other previously obtained value. The reference value may be determined concurrently with the methods disclosed herein, or may have been determined previously.

Control subjects from which samples are/have been obtained may have undergone treatment for a complement-related disorder and/or received a complement-related therapy/therapeutic agent.

Controls may be positive controls in which the target molecule is known to be present, or expressed at high level, or negative controls in which the target molecule is known to be absent or expressed at low level.

Samples, e.g. from one or more control subjects, may comprise any one, two, three, four, five, six, seven, eight, nine, ten, eleven, twelve, thirteen, fourteen, fifteen or sixteen of FHR1, FHR2, FHR3, FHR4, FHR5, FH, FHL-1, FI, C3, C3b, C3a, iC3b, C3f, C3c, C3dg, and/or C3d, or any combination thereof. In some cases each complement protein is in a separate control sample. In some cases a control sample contains multiple complement proteins. In some cases the methods described herein comprise comparing the level of one of more complement proteins determined as described herein to different, e.g. one or more, samples, each sample containing one or more complement proteins. In some cases the methods described herein comprise comparing the level of one or more complement proteins determined as described herein to a single sample, wherein the sample contains one or more complement proteins.

In some cases control samples are obtained from the same tissue(s) as the sample obtained from the individual to be tested. In some cases control samples are obtained from different tissue(s) as the sample obtained from the individual to be tested. Control samples may be obtained from control subjects at certain time(s) of day, or on certain days. Sample(s) obtained from the individual to be tested are preferably obtained at the same time(s) of day and/or day(s) as the control samples.

In some embodiments the methods comprise detecting/determining the level of a complement protein in a sample. The sample may be in vitro or ex vivo. A sample may have been taken from a subject. A sample may be taken from any tissue or bodily fluid. In preferred arrangements the sample is taken from a bodily fluid, more preferably one that circulates through the body. Accordingly, the sample may be a blood sample or lymph sample. In a particularly preferred arrangement the sample is a blood sample or blood-derived sample. The blood-derived sample may be a selected fraction of a patient or subject's blood, e.g. a selected cell-containing fraction or a plasma or serum fraction. A selected serum fraction may comprise the fluid portion of the blood obtained after removal of the fibrin clot and blood cells. Alternatively the sample may comprise or may be derived from a tissue sample, biopsy or isolated cells from said individual. The sample may be taken from the eye, kidney, brain or liver, e.g. comprising cells from the eye, kidney, brain or liver. The sample may comprise retinal tissue. The sample may comprise RPE cells or tissue from Bruch's membrane or the choroid. The sample may comprise drusen or other deposits of complement-related components.

In some embodiments the methods described herein comprise taking or obtaining a sample from a subject, e.g. blood, tissue etc. In some embodiments the methods described herein are performed on a sample that has been obtained/was obtained from a subject. In some cases the sample is a blood sample. The blood sample may undergo/have undergone processing to obtain a plasma sample or a serum sample. In some cases, the methods comprise obtaining a blood-derived sample from a subject. In some cases, the methods comprise obtaining a plasma or serum sample from a subject. In some embodiments the methods comprise isolating protein, e.g. total protein, from the sample. Suitable techniques to isolate protein from biological samples are well known in the field. In some embodiments the methods do not comprise isolating protein from the sample, e.g. the methods are performed on the unprocessed sample.

In some embodiments, the methods are performed in vitro. For example, the presence, level, amount and/or concentration of the complement protein(s) may be detected/determined in vitro.

In some cases the methods involve determining the presence, level, amount and/or concentration of the complement protein(s) in a subject. This may involve performing the methods described herein in vitro, and using the results to calculate the presence, level, amount and/or concentration of the protein(s) in the subject.

Also provided is a method for detecting at least one complement protein in a sample, the method comprising digesting the protein(s) in the sample with endoproteinase GluC to obtain one or more peptides; and using mass spectrometry to detect the one or more peptides in the sample.

Also provided is a method for determining the level of at least one complement protein in a sample, the method comprising digesting the protein(s) in the sample with endoproteinase GluC to obtain one or more peptides and using mass spectrometry to determine the level of the one or more peptides in the sample.

Using mass spectrometry to detect one or more peptides in a sample, or detecting and/or determining the level of one or more peptides by mass spectrometry, e.g. by the methods described herein, may include applying a mass spectrometry technique to the sample, e.g. by putting the sample in a mass spectrometer, and instructing the mass spectrometer to analyse the sample. Various suitable mass spectrometry techniques are disclosed herein and are within the routine tasks of a skilled person.

In any aspect provided herein, the methods described herein may comprise both detecting at least one complement protein and determining the level of at least one complement protein. The complement protein may be the same protein, and/or the methods may comprise detecting a least a first complement protein and determining the level of at least a second complement protein.

In some embodiments, the methods described herein comprise detecting/determining the level of one complement protein. In some embodiments, the methods described herein comprise detecting/determining the level of at least one complement protein, one or more complement proteins, and/or groups or complement proteins e.g. as provided herein.

In some embodiments the complement protein is encoded from the RCA (regulators of complement) gene cluster, or RCA locus, on human chromosome 1. The RCA cluster is located on chromosome 1q32 and includes the CFH and CFHR1-5 genes. The gene cluster also includes the membrane bound proteins CR1 (CD35), CR2 (CD21), decay-accelerating factor (DAF; CD55), and membrane cofactor protein (MCP; CD46), as well as soluble C4b-binding protein (C4 bp).

The methods described herein are suitable for detecting/determining the level of multiple complement proteins via a single assay: i.e. using a single enzyme, GluC, to obtain analysable peptides and then using a single analytical technique, mass spectrometry, to detect and/or determine the levels of said peptides. In this way, the complementome of a sample or a subject can be determined via a single assay.

In some embodiments the methods described herein comprise detecting/determining the level of any one or more, e.g. any or all combinations, of FH, FHL-1, FHR1, FHR2, FHR3, FHR4, and/or FHR5. In some embodiments the complement protein(s) is/are selected from the group consisting of FH, FHL-1, FHR1, FHR2, FHR3, FHR4, and/or FHR5. In some cases the methods comprise detecting/determining the level of any one, two, three, four, five, six and/or seven of FH, FHL-1, FHR1, FHR2, FHR3, FHR4, and FHR5, alone or in combination. In some cases the methods described herein are able to differentiate (i.e. distinguish, discriminate, separate) between the presence of (or levels of) each of FH, FHL-1, FHR1, FHR2, FHR3, FHR4 and/or FHR5.

In some cases the complement protein to be detected/determine the level of is FH and/or FHL-1. In some cases the methods described herein comprise detecting/determining the level of both FH and FHL-1. In some cases the methods described herein differentiate (i.e. distinguish, discriminate, separate) between the presence of FH and the presence of FHL-1 and/or between the level/concentration of FH and the level/concentration of FHL-1. In some cases the methods described herein permit or allow the detection of FH alone, i.e. without detecting FHL-1. In some cases the methods described herein permit or allow the detection of FHL-1 alone, i.e. without detecting FH.

In some cases the complement protein is any one or more, e.g. any or all combinations, of FHR1, FHR2, FHR3, FHR4 and/or FHR5. In some cases the complement protein is FHR4. In some cases the methods described herein are able to differentiate (i.e. distinguish, discriminate, separate) between the presence of (or levels of) each of FHR1, FHR2, FHR3, FHR4 and/or FHR5. In some cases, the methods described herein permit or allow the detection of FHR1 alone, i.e. without detecting FHR2-FHR5. In some cases, the methods described herein permit or allow the detection of FHR2 alone, i.e. without detecting FHR1 or FHR3-FHR5. In some cases, the methods described herein permit or allow the detection of FHR3 alone, i.e. without detecting FHR1, FHR2, FHR4, or FHR5. In some cases, the methods described herein permit or allow the detection of FHR4 alone, i.e. without detecting FHR1-FHR3 or FHR5. In some cases, the methods described herein permit or allow the detection of FHR5 alone, i.e. without detecting FHR1-FHR4.

In some embodiments the complement protein to be detected/the level of which is determined is involved with breakdown, turnover and/or inactivation of C3/C3b. In some embodiments, the complement protein is produced by the breakdown and/or inactivation of C3/C3b, i.e. is a product of C3b inactivation/breakdown. In some embodiments the methods described herein include determining the presence, rate and/or progression of C3b turnover. In some embodiments the methods described herein involve detecting/determining the level of a protein involved in, or produced as a result of, the complement amplification loop. In some embodiments the methods described herein involve detecting/determining the level of a protein involved in the generation or breakdown of C3 convertase. In some cases the protein is a cofactor for FI, e.g. FH, CR1, some of the FHR proteins. Any method disclosed herein, e.g. a method for detecting at least one complement protein in a sample comprising digesting proteins with GluC and detecting the resulting peptides by mass spectrometry, may be described in the alternative as a method for detecting C3 turnover, a method for detecting C3 breakdown, a method for measuring C3b turnover or C3b breakdown, or a method for measuring the progress of C3b turnover or C3b breakdown.

Thus, in some aspects the present invention provides a method for detecting turnover or breakdown of C3b, comprising the steps described herein, e.g. digesting at least one complement protein with endoproteinase GluC to obtain one or more peptides and detecting the peptide(s) by mass spectrometry. In some cases the method comprises digesting and then detecting at least two, three, four or more, up to 16, of the 16 complement proteins described herein.

In some embodiments the methods described herein comprise/further comprise detecting/determining the level of FI, either alone or in combination with other complement proteins such as those described herein.

In some embodiments the methods described herein comprise detecting/determining the level of any one or more, e.g. any or all combinations, of C3, C3b, C3a, iC3b, C3f, C3c, C3dg, and/or C3d. In some embodiments the complement protein(s) is/are selected from the group consisting of C3, C3b, C3a, iC3b, C3f, C3c, C3dg, and/or C3d. In some cases the methods comprise detecting/determining the level of any one, two, three, four, five, six, seven and/or eight of C3, C3b, C3a, iC3b, C3f, C3c, C3dg, and/or C3d, in any combination. In some embodiments the methods described herein comprise detecting/determining the level of one or more of C3, C3a, C3f, C3c, and/or C3d. In some cases the methods described herein comprise determining the presence and/or level of C3b, iC3b, and/or C3dg, e.g. via the methodology in Table 3. In some cases the methods described herein comprise detecting/determining the level of C3, C3b and/or iC3b. In some cases the methods described herein are able to differentiate (i.e. distinguish, discriminate, separate) between the presence of (or levels of) two or more, or all, of C3, C3b, C3a, iC3b, C3f, C3c, C3dg, and/or C3d.

The methods described herein can detect multiple complement proteins, and distinguish between said complement proteins, using one enzyme e.g. GluC and one analytical method e.g. mass spectrometry. The methods described herein may be used to detect/determine the level of any one of the individual proteins described herein, as well as any and all combinations of FH, FHL-1, FHR1, FHR2, FHR3, FHR4, FHR5, FI, C3, C3b, C3a, iC3b, C3f, C3c, C3dg, and/or C3d, i.e. any one, two, three, four, five, six, seven, eight, nine, ten, eleven, twelve, thirteen, fourteen, fifteen and/or sixteen of these proteins in any combination. In some embodiments the complement protein(s) is/are selected from the group consisting of FH, FHL-1, FHR1, FHR2, FHR3, FHR4, FHR5, FI, C3, C3b, C3a, iC3b, C3f, C3c, C3dg, and/or C3d. In some cases the methods described herein may be used to detect/determine the level of FHL-1 and to detect/determine the level of any one or more of FH, FHR1, FHR2, FHR3, FHR4, FHR5, FI, C3, C3b, C3a, iC3b, C3f, C3c, C3dg, and/or C3d. In some cases the method comprises distinguishing (i.e. differentiating, discriminating, separating) between the presence/level of FHL-1 and the presence/level of any one or more of FH, FHR1, FHR2, FHR3, FHR4, FHR5, FI, C3, C3b, C3a, iC3b, C3f, C3c, C3dg, and/or C3d. The terms “distinguishing”, “differentiating”, “discriminating”, and “separating” are used interchangeably herein.

In some cases the methods provided herein allow for simultaneous detection of one or more of FH, FHL-1, FHR1, FHR2, FHR3, FHR4, FHR5, FI, C3, C3b, C3a, iC3b, C3f, C3c, C3dg, and/or C3d, including any combination thereof. In some cases the methods provided herein allow for detection/determination of the level of one or more of FH, FHL-1, FHR1, FHR2, FHR3, FHR4, FHR5, FI, C3, C3b, C3a, iC3b, C3f, C3c, C3dg, and/or C3d, including any combination thereof, in a single assay. The methods provided herein allow for distinct, separable and detectable peptides to be produced from every protein listed above such that the presence and/or level of each protein can be distinguished from the others.

In some cases, the present invention provides a method for detecting and/or determining the level of at least two complement proteins in a sample simultaneously and/or in one assay, the method comprising:
digesting the proteins with endoproteinase GluC to obtain one or more peptides; and
detecting and/or determining the level of the one or more peptides by mass spectrometry.

In any method described herein, the complement protein may be any protein involved in one or more of the complement system pathways. For example, the complement protein may be one or more of C1, C2, C4b2a C4, C4a, C5, C5a, FB, FD, C3Bb, MASP1, MASP2, C1q, C1r, Cis, C6, C7, C8, C9, CD59, Clusterin, Properdin, and/or Compstatin. In any embodiment described herein, the complement protein to be detected (or the protein whose level is determined) is not one or more of C1, C2, C4b2a C4, C4a, C5, C5a, FB, FD, C3Bb, MASP1, MASP2, C1q, C1r, C1s, C6, C7, C8, C9, CD59, Clusterin, Properdin, and/or Compstatin.

In some cases, the present invention uses endoproteinase GluC for preparing at least one complement protein for detection by mass spectrometry. In some cases the invention uses endoproteinase GluC for preparing at least two, i.e. multiple or a plurality of, complement proteins for detection by mass spectrometry. The at least two complement proteins may be any two, three, four or more, up to 16, of FH, FHL-1, FHR1, FHR2, FHR3, FHR4, FHRS, FI, C3, C3b, C3a, iC3b, C3f, C3c, C3dg, and/or C3d, in any combination, as described herein.

Endoproteinase GluC, also known as glutamyl endopeptidase, is a serine proteinase which preferentially cleaves peptide bonds C-terminal to glutamic acid residues. It also cleaves at aspartic acid residues at a rate 100-300 times slower than at glutamic acid residues. The specificity of GluC depends on the pH and the buffer composition. At pH 4, the enzyme preferentially cleaves at the C terminus of E, whereas at pH 8 it additionally cleaves at D residues. The sequence of GluC is provided in SEQ ID NO:153 and 154.

In preferred embodiments, the methods described herein use GluC alone (i.e. only GluC) to digest the one or more complement proteins. In preferred embodiments, a step of digesting the protein(s) in the described methods consists of digesting the protein(s) with GluC. In preferred embodiments, any method described herein does not employ/use any other protease alone or in combination with GluC. For example, in some embodiments the digestion step of any method described herein does not use, or is not performed by, any one or more of the following enzymes or agents: trypsin, chymotrypsin (high specificity or low specificity), Lys-C, Lys-N, Arg-C, Asp-N, elastase, LysargiNase, pepsin, Sap9, OmpT, BNPS-skatole, any caspase, clostripain (clostridiopeptidase B), CNBr, enterokinase, factor Xa, granzymeB, neutrophil elastase, proteinase K, thermolysin, non-GluC glutamyl endopeptidase e.g. GluBI or GluSGB, proline endopeptidase, TEV protease, thrombin, formic acid, hydroxylamine, iodosobenzoic acid, and/or NTCB (or any combination thereof).

GluC is obtainable from standard reagent providers e.g. Sigma Aldrich, NEB etc, and may be used according to the accompanying instructions or according to protocols well known in the field. An example protocol is described herein. Obtaining proteins from biological samples and suitable buffers to prepare samples/proteins for GluC digestion will also be known to the skilled person. An example cell lysis buffer comprises: 8 M urea (4.8 g per 10 ml) in 50 mM NH₄HCO₃ and 20 mM methylamine, diluted to a urea concentration of <2 M, pH 8 (40 mg per 10 ml), containing 1 tablet of cOmplete 1 ™ Mini EDTA-free protease inhibitor cocktail per 10 ml of lysis buffer.

In some cases, a complement protein is contacted/incubated/digested with GluC enzyme for at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, or at least 24 hours. In some cases a complement protein is contacted/incubated/digested with GluC enzyme for at least 12 hours. In some cases a complement protein is contacted/incubated/digested with GluC enzyme for about 12 hours, e.g. 12 hours. In some cases a complement protein is contacted/incubated/digested with GluC enzyme for about 16 hours, e.g. 16 hours. The terms contacted, incubated and digested are used interchangeably herein.

In some cases a complement protein is contacted/incubated/digested with GluC enzyme at a temperature of at least 20° C., at least 21° C., at least 22° C., at least 23° C., at least 24° C., at least 25° C., at least 26° C., at least 27° C., at least 28° C., at least 29° C., or at least 30° C. In some cases a complement protein is contacted/incubated/digested with GluC enzyme at a temperature of at least In some cases a complement protein is contacted/incubated/digested with GluC enzyme at a temperature of about 25° C., e.g. 25° C.

In some cases a complement protein is contacted/incubated/digested with GluC enzyme at a pH of at least 7.0, at least 7.1, at least 7.2, at least 7.3, at least 7.4, at least 7.5, at least 7.6, at least 7.7, at least 7.8, at least 7.9, at least 8.0, at least 8.1, at least 8.2, at least 8.3, at least 8.4, at least 8.5, at least 8.6, at least 8.7, at least 8.8, at least 8.9, or at least 9.0. In some cases a complement protein is contacted/incubated/digested with GluC enzyme at a pH of at least 8.0. In some cases a complement protein is contacted/incubated/digested with GluC enzyme at a pH of about 8.0, e.g. a pH of 8.0.

In some cases the GluC enzyme and complement protein are contacted/incubated at a wt/wt ratio of 1/75. The incubation step may comprise gentle shaking, e.g. at 400 rpm.

The methods described herein may comprise a contacting/incubation/digestion step comprising any combination of temperature, pH, and/or time as described above. In some cases, contacting/incubating/digesting is performed at 25° C. at pH8 for 12 hours.

In some embodiments the invention provides a method for detecting and/or determining the level of at least one complement protein e.g. in a sample, the method comprising:
digesting the protein(s) with endoproteinase GluC to obtain one or more peptides, the digesting comprising incubating the protein(s) with GluC at 25° C. at pH8 for up to 12 hours; and detecting the one or more peptides by mass spectrometry.

In some embodiments the invention provides a method for detecting and/or determining the level of at least one complement protein e.g. in a sample, the method comprising:
digesting the protein(s) with endoproteinase GluC to obtain one or more peptides, the digesting comprising incubating the protein(s) with GluC at 25° C. at pH8 for up to 16 hours; and
detecting the one or more peptides by mass spectrometry.

The following peptides may be produced by GluC digestion of complement proteins, e.g. as described herein. In some embodiments, the methods described herein comprise detecting/determining the level of any one or more of these peptides, i.e. any one or more of SEQ ID NO:20 to 141, or 155, 156 or 157, in any combination. All combinations of peptides are envisaged. The mass of peptides represented by SEQ ID NOs 20-27 can be found in Table 1.

In some embodiments, the FH peptide is VTYKCFE (SEQ ID NO:20).

In some embodiments the FH peptide is any one or more of SNTGSTTGSIVCGYNGWSDLPICYE (SEQ ID NO:112; mass 2623.1206), NGWSPTPRCIRVKTCSKSSIDIE (SEQ ID NO:113; mass 2576.2839), LPKIDVHLVPDRKKDQYKVGE (SEQ ID NO:114; mass 2476.3801), YYCNPRFLMKGPNKIQCVDGE (SEQ ID NO:115; mass 2474.1545), NYNIALRWTAKQKLYSRTGE (SEQ ID NO:116; mass 2411.2709), KWSHPPSCIKTDCLSLPSFE (SEQ ID NO:117; mass 2274.0813), HGWAQLSSPPYYYGDSVE (SEQ ID NO:118; mass 2054.9010), ISHGWAHMSDSYQYGEE (SEQ ID NO:119; mass 2007.8632), FDHNSNIRYRCRGKE (SEQ ID NO:120; mass 1893.9016), ITCKDGRWQSIPLCVE (SEQ ID NO:121; mass 1846.9069), GWIHTVCINGRWDPE (SEQ ID NO:122; mass 1781.8307), KAKYQCKLGYVTADGE (SEQ ID NO:123; mass 1772.8767), TTCYMGKWSSPPQCE (SEQ ID NO:124; mass 1716.6946), SYAHGTKLSYTCE (SEQ ID NO:125, mass 1458.6449), RVRYQCRSPYE (SEQ ID NO:126; mass 1455.7041), GFGIDGPAIAKCLGE (SEQ ID NO:127; mass 1446.7176), HGTINSSRSSQE (SEQ ID NO:128; mass 1301.5960), YQCQNLYQLE (SEQ ID NO:129; mass 1300.5758), WTTLPVCIVEE (SEQ ID NO:130; mass 1288.6373), KIPCSQPPQIE (SEQ ID NO:131; mass 1238.6329), SQYTYALKE (SEQ ID NO:132; mass 1101.5342), QVQSCGPPPE (SEQ ID NO:133; mass 1040.4597), KKDVYKAGE (SEQ ID NO:134; mass 1036.5553), GLPCKSPPE (SEQ ID NO:135; mass 926.4531), KVSVLCQE (SEQ ID NO:136; mass 904.4688), HLKNKKE (SEQ ID NO:137; mass 895.5239), GGFRISEE (SEQ ID NO:138; mass 893.4243), LLNGNVKE (SEQ ID NO: 139; mass 885.4920), YPTCAKR (SEQ ID NO:140; mass 837.4167), or STCGDIPE (SEQ ID NO:141; mass 820.3273).

In some embodiments, the FHL-1 peptide is NGWSPTPRCIRVSFTL (SEQ ID NO:21).

In some embodiments, the FHR1 peptide is ATFCDFPKINHGILYGEE (SEQ ID NO:22).

In some embodiments the FHR1 peptide is NYNIALRWTAKQKLYLRTGE (SEQ ID NO:91; mass 2437.3230).

In some embodiments the FHR2 peptide is RGWSTPPKCRSTISAE (SEQ ID NO:23).

In some embodiments the FHR2 peptide is AMFCDFPKINHGILYDEE (SEQ ID NO:24). In some embodiments the FHR2 peptide is YNFVSPSKSFWTRITCAEE (SEQ ID NO:92; mass 2264.0572).

In some embodiments the FHR3 peptide is VACHPGYGLPKAQTTVTCTE (SEQ ID NO:25).

In some embodiments the FHR3 peptide is any one or more of KGWSPTPRCIRVRTCSKSDIE (SEQ ID NO:93; mass 2418.2260), NGYNQNYGRKFVQGNSTE (SEQ ID NO:94; mass 2074.9457), QVKPCDFPDIKHGGLFHE (SEQ ID NO:95; mass 2066.0043), FMCKLGYNANTSILSFQAVCRE (SEQ ID NO:96; mass 2494.1807), or YQCQPYYE (SEQ ID NO:97; mass 1092.4222).

In some embodiments the FHR4 peptide is YQCQSYYE (SEQ ID NO:26).

In some embodiments the FHR4 peptide is any one or more of NSRAKSNGMRFKLHDTLDYE (SEQ ID NO: 98; mass 2381.1546), DGWSHFPTCYNSSE (SEQ ID NO:99; mass 1628.6202), ISYGNTTGSIVCGE (SEQ ID NO:100; mass 1399.6289), or FMCKLGYNANTSVLSFQAVCRE (SEQ ID NO:101; mass 2480.1650).

In some embodiments the FHR5 peptide is RGWSTPPICSFTKGE (SEQ ID NO:27).

In some embodiments the FHR5 peptide is any one or more of GTLCDFPKIHHGFLYDEE (SEQ ID NO:102; mass 2119.9673), YAMIGNNMITCINGIWTE (SEQ ID NO:103; mass 2042.9264), YGYVQPSVPPYQHGVSVE (SEQ ID NO:104; mass 2004.9581), GDTVQIICNTGYSLQNNE (SEQ ID NO:105; mass 1967.8895), IVCKDGRWQSLPRCVE (SEQ ID NO:106; mass 1887.9447), DYNPFSQVPTGE (SEQ ID NO:107; mass 1352.5884), QVKTCGYIPE (SEQ ID NO:108; mass 1136.5536), ANVDAQPKKE (SEQ ID NO:109; mass 1098.5669), WTTLPTCVE (SEQ ID NO:110; mass 1048.4899), or KVAVLCKE (SEQ ID NO:111; mass 888.5102).

In some cases, the methods described herein comprise detecting/determining the level of one or more of SEQ ID NOs 21-27, in any combination.

In some cases, any method described herein may comprise detecting/determining the level of one or more of SEQ ID NOs 28-37, 156 or 157, in any combination. In some cases, the methods provided herein are used to detect C3, C3b and breakdown products using one or more or all of the peptides in Table 2 in any combination, plus optionally SEQ ID NO:156 and/or 157, for example according to the methodology in Table 3.

In some embodiments the FI peptide is any one or more of VKLVDQDKTMFICKSSWSMRE (SEQ ID NO:45; mass 2531.2455), VKLISNCSKFYGNRFYE (SEQ ID NO:46; mass 2068.0320), CLHPGTKFLNNGTCTAE (SEQ ID NO:47; mass 1805.8309), NYNAGTYQNDIALIE (SEQ ID NO:48; mass 1698.7969), GKFSVSLKHGNTDSE (SEQ ID NO:49; mass 1605.7867), VGCAGFASVTQEE (SEQ ID NO:50; mass 1297.5729), VGCAGFASVTQE (SEQ ID NO:155; mass 1168.272), MKKDGNKKDCE (SEQ ID NO:51; mass 1295.6082), YVDRIIFHE (SEQ ID NO:52; mass 1191.6156), CLHVHCRGLE (SEQ ID NO:53; mass 1166.5557), RVFSLQWGE (SEQ ID NO:54; mass 1121.5738), ILTADMDAE (SEQ ID NO:55; mass 978.4448), or KVTYTSQE (SEQ ID NO:56; mass 955.4731).

In some embodiments the FI peptide is any one or more of CAGTYDGSIDACKGDSGGPLVCMDANNVTYVWGVVSWGE (SEQ ID NO:38; mass 3996.7183), GTCVCKLPYQCPKNGTAVCATNRRSFPTYCQQKSLE (SEQ ID NO:39; mass 3994.8853), FPGVYTKVANYFDWISYHVGRPFISQYNV (SEQ ID NO:40; mass 3467.7211), ANVACLDLGFQQGADTQRRFKLSDLSINSTE (SEQ ID NO:41, mass 3397.6804), LPRSIPACVPWSPYLFQPNDTCIVSGWGRE (SEQ ID NO:42, mass 3388.6605), KKCLAKKYTHLSCDKVFCQPWQRCIE (SEQ ID NO:43; mass 3155.5773), LCCKACQGKGFHCKSGVCIPSQYQCNGE (SEQ ID NO:44; mass 2991.2861), VKLVDQDKTMFICKSSWSMRE (SEQ ID NO:45; mass 2531.2455), VKLISNCSKFYGNRFYE (SEQ ID NO:46; mass 2068.0320), CLHPGTKFLNNGTCTAE (SEQ ID NO:47; mass 1805.8309), NYNAGTYQNDIALIE (SEQ ID NO:48; mass 1698.7969), GKFSVSLKHGNTDSE (SEQ ID NO:49; mass 1605.7867), VGCAGFASVTQEE (SEQ ID NO:50; mass 1297.5729), MKKDGNKKDCE (SEQ ID NO:51; mass 1295.6082), YVDRIIFHE (SEQ ID NO:52; mass 1191.6156), CLHVHCRGLE (SEQ ID NO:53; mass 1166.5557), RVFSLQWGE (SEQ ID NO:54; mass 1121.5738), ILTADMDAE (SEQ ID NO:55; mass 978.4448), KVTYTSQE (SEQ ID NO:56; mass 955.4731), VDCITGE (SEQ ID NO:57; mass 736.3182), NCGKPE (SEQ ID NO:58; mass 647.2817), TSLAE (SEQ ID NO:59; mass 520.2613), or KDNE (SEQ ID NO:60; mass 505.2252).

Peptides detected by the methods described herein may optionally have at least 70%, preferably one of 80%, 85%, 90%, 91%, 92%, 93%, 94%, 95%, 96%, 97%, 98%, 99% or 100% amino acid sequence identity to the amino acid sequences of the peptides described herein, e.g. any one of SEQ ID NOs 21-141. Other suitable peptides may be readily determined by a skilled person and may be employed in the methods described herein. In preferred embodiments, the peptides used according to the methods herein permit mass spectrometry techniques to distinguish or differentiate between two or more complement proteins in a sample.

### Mass Spectrometry

Methods provided herein comprising detecting and/or determining the levels of proteins may involve using mass spectrometry to detect and/or determine the levels of proteins in a sample.

In preferred embodiments, any method described herein involves using only mass spectrometry (i.e. mass spectrometry alone) to detect/determine the level of the one or more peptides. That is, in some embodiments, the methods provided herein do not employ multiple analytical techniques and the peptide(s) are detected/determined/measured using a single assay. In preferred embodiments, the methods described herein do not detect/determine the level of/measure the peptide(s) using mass spectrometry in combination with another analytical technique suitable for detecting proteins/peptides. In preferred embodiments, detection/determination of the level of the one or more peptides is not performed at any stage using a non-mass spectrometry technique, e.g. detection/determination of the level of the peptide(s) is not performed using high performance liquid chromatography (HPLC), immunological-based methods such as quantitative enzyme-linked immunosorbent assays (ELISA), Western blotting, protein immunoprecipitation, dot blotting or immunoelectrophoresis, electrophoresis or autoradiography. In some cases, liquid chromatography-mass spectrometry (LC/MS) is not used.

As used herein, “detecting and/or determining the level of” e.g. a complement protein or peptide “by mass spectrometry” is the same as “using mass spectrometry to detect and/or determine the level of” e.g. a complement protein or peptide.

Mass spectrometry is a well-known analytical technique for analysing a sample that typically comprises generating ions from the sample, optionally fragmenting the ions, separating the ions according to their mass/charge ratio (in time and/or space), and detecting the ions to provide information regarding the content of the sample.

For the purpose of detecting and/or determining the levels of proteins, at least one fragmentation step may be included.

Mass spectrometry techniques are well-known in the field and any suitable mass spectrometry technique may be employed for detecting and/or determining the levels of proteins in a sample, e.g. LC/MS, GC/MS, tandem mass spectrometry (MS/NIS), quadrupole MS e.g. triple quadrupole MS (TQMS), time-of-flight MS e.g. MALDI-TOF, targeted MS e.g. selected reaction monitoring MS (SRM-MS)/multiple reaction monitoring (MRM-MS), parallel-reaction monitoring (PRM-MS), trapped-ion based methods e.g. three-dimensional quadrupole ion traps (“dynamic” traps) and ion cyclotron resonance mass spectrometers (“static” traps), quadrupole trap MS, hybrid linear trap orbitrap MS, quadrupole-Orbitrap MS, electrospray Ionization mass spectrometry (ESI-MS), or electron transfer dissociation MS (ETD).

In some embodiments, the mass spectrometry technique may be a liquid chromatography-selected reaction monitoring mass spectrometry (LC-SRM-MS)-based assay.

Fragmenting the ions may be achieved using any suitable fragmentation technique, e.g. collision-induced dissociation (CID)/collisionally activated dissociation (CAD), electron-capture dissociation (ECD), electron transfer dissociation (ETD), in-source decay (ISD), infrared multiple photon dissociation (IRMPD) etc. Again, such techniques are well known.

The mass spectrometry techniques useful in the present invention may comprise quantitative analysis. Mass spectrometry methods comprising quantitative analysis may comprise a targeted approach to detect and measure peptides of interest and their corresponding fragments. This may allow for greater specificity and sensitivity for quantification. Quantitative mass spectrometry in proteomics is reviewed in e.g. Bantscheff, M., et al. Anal Bioanal Chem 2007, 389, 1017-1031, which is hereby incorporated by reference in its entirety.

For example, input peptides may undergo fragmentation in a collision cell, thus generating product ions exclusive to the peptides. Both the intact peptide mass and one or more specific fragment ions of that peptides can be monitored over the course of an MS experiment e.g. using SRM/MRM, PRM etc.

The observed m/z ratio of a peptide and its corresponding product ion m/z ratio are referred to as a “transition”, i.e. a mass pair representing the m/z of an analyte (the parent ion) and the m/z of one of its product ions which is formed upon fragmentation of the parent ion.

It is well within the routine work of a skilled person to develop suitable transitions for quantitative mass spectrometry techniques, e.g. SRM/MRM-MS and PRM-MS. Mead et al., Mol Cell Proteomics. 2009 Apr; 8(4): 696-705, hereby incorporated by reference in its entirety, describes one such technique for designing transitions.

Tables 7 and 8 provide examples of transitions for the complement proteins described herein, based on fragmentation of synthetic versions of each peptide of interest. Suitable alternative transitions may also be used, the identification of which is well within the routine remit of a skilled person.

Quantitation can be achieved by ‘spiking’ the sample with known quantities of labelled synthetic peptides. The combination of retention time, peptide mass, and fragment mass practically eliminates ambiguities in peptide assignments and extends the quantification range to 4-5 orders of magnitude. In some cases, the methods provided herein comprise a step of determining optimised MS settings and/or quantitation reference values using stable isotopic standards.

Mass spectrometry techniques that may be used in the present invention may comprise targeted or semi-targeted MS workflows and/or data-dependent acquisition (DDA) or data-independent acquisition (DIA) techniques.

DDA uses knowledge obtained during the acquisition to decide which MS1 peptide precursors to subject for fragmentation (MS/MS) in the collision cell. DIA, in contrast, performs predefined MS/MS fragmentation and data collection regardless of sample content, which allows for more sensitive and accurate protein quantification compared to DDA. DIA strategies can be further segregated into targeted or untargeted acquisitions. Targeted DIA methods fragment predefined precursor ions that correspond to the peptide analytes, usually at known (measured or predicted) retention times. Targeted DIA has become widely used in academic, pharmaceutical, and biotechnology research for quantification of small molecules (metabolites), peptides, and post-translational modifications (PTMs). For example, selected-reaction monitoring (SRM), a type of targeted DIA, is currently considered the gold standard method for mass spectrometric quantification due to its high accuracy and precision. For a review of DIA techniques, see e.g. Meyer and Schilling, Expert Rev Proteomics. 2017 May; 14(5): 419-429, hereby incorporated in its entirety.

Other suitable DIA methods include e.g. Sequential Window Acquisition of All Theoretical mass spectrometry (SWATH MS; see e.g. Ludwig et al., Mol Syst Biol (2018)14:e8126), SONAR (Waters.com), or Online Parallel Accumulation-Serial Fragmentation (PASEF; see e.g. Meier et al., J Proteome Res. 2015 Dec 4;14(12):5378-87 and Meier et al., Mol Cell Proteomics. 2018 Dec; 17(12): 2534-2545).

### Applications of the Methods

In some aspects the invention provides methods for assessing the risk of onset or risk of progression of a complement-related disorder using the detection/determination methods described herein.

The methods for assessing risk may be diagnostic, prognostic and/or predictive of the risk of onset or progression of a complement-related disorder. Diagnostic methods can be used to determine the diagnosis or severity of a disease, prognostic methods help to predict the likely course of disease in a defined clinical population under standard treatment conditions, and predictive methods predict the likely response to a treatment in terms of efficacy and/or safety, thus supporting clinical decision-making. The methods described herein may be useful in monitoring the success of treatment, including past or ongoing treatment, for complement-related disorders.

The terms “disorder”, “disease” and “condition” may be used interchangeably and refer to a pathological issue of a body part, organ or system which may be characterised by an identifiable group of signs or symptoms. The term “complement-related disorder” refers to disorders, diseases or conditions that comprise or arise from deficiencies or abnormalities in the complement system. In some embodiments, the complement-related disorder is a disorder driven by complement activation or complement over-activation. The terms “develop”, “developing”, and “development”, e.g. of a disorder, as used herein refer both to the onset of a disease as well as the progression, exacerbation or worsening of a disease state.

In some embodiments the disorder is one in which the complement system, or activation/over-activation/dysregulation thereof, is pathologically implicated. The complement related disorder may be any disorder described herein. “Pathologically implicated” as used herein may refer to a protein level which is raised or lowered in the disorder compared with a reference value, and/or where the protein contributes towards the pathology of the disorder. The selection or combination of complement protein(s) detected may depend on the complement-related disorder of interest and the complement protein(s) that are useful biomarkers for said disorder.

The complement-related disorder may comprise disruption of the classical, alternative and/or lectin complement pathways. In some cases, the disorder may be associated with deficiencies in, abnormalities in, or absence of regulatory components of the complement system. In some embodiments, the disorder may be a disorder associated with the alternative complement pathway, disruption of the alternative complement pathway and/or associated with deficiencies in, abnormalities in, or absence of regulatory components of the alternative complement pathway. In some cases the disorder is associated with the complement amplification loop. In some cases the disorder is associated with inappropriate activation, over-activation, or dysregulation of the complement system, in whole or in part, e.g. C3 convertase assembly, C3b production, C3b deposition, and/or the amplification loop.

In some cases, the disorder is associated with any one or more of C3, C3b, iC3b, FI, FH, FHL-1, or FHR1-FHR5. In some cases, the disorder is associated with deficiencies or abnormalities in the activity of any one or more of C3, C3b, iC3b, FI, FH, FHL-1, or FHR1-FHR5. In some cases one or more of these proteins are pathologically implicated in the disorder, e.g. have raised or lower levels compared with a reference value.

In some cases, the disorder is associated with one or more of CR1, CD46, CD55, C4BP, Factor B (FB), Factor D (FD), SPICE, VCP (or VICE) and/or MOPICE. In some cases, the disorder is associated with deficiencies or abnormalities in the activity of one or more of CR1, CD46, CD55, C4BP, Factor B, Factor D, SPICE, VCP (or VICE) and/or MOPICE, or where one or more of these proteins are pathologically implicated.

In some embodiments, the disorder may be a disorder associated with C3 or a C3-containing complex, an activity/response associated with C3 or a C3-containing complex, or a product of an activity/response associated with C3 or a C3-containing complex. That is, in some embodiments, the disorder is a disorder in which C3, a C3-containing complex, an activity/response associated with C3 or a C3-containing complex, or the product of said activity/response is pathologically implicated. In some embodiments, the disorder may be associated with an increased level of C3 or a C3-containing complex, an increased level of an activity/response associated with C3 or a C3-containing complex, or an increased level of a product of an activity/response associated with C3 or a C3-containing complex as compared to the control state. In some embodiments, the disorder may be associated with a decreased level of C3 or a C3-containing complex, a decreased level of an activity/response associated with C3 or a C3-containing complex, or a decreased level of a product of an activity/response associated with C3 or a C3-containing complex as compared to the control state.

In some embodiments, the disorder may be a disorder associated with C3b or a C3b-containing complex, an activity/response associated with C3b or a C3b-containing complex, or a product of an activity/response associated with C3b or a C3b-containing complex. That is, in some embodiments, the disorder is a disorder in which C3b, a C3b-containing complex, an activity/response associated with C3b or a C3b-containing complex, or the product of said activity/response is pathologically implicated. In some embodiments, the disorder may be associated with an increased level of C3b or a C3b-containing complex, an increased level of an activity/response associated with C3b or a C3b-containing complex, or increased level of a product of an activity/response associated with C3b or a C3b-containing complex as compared to the control state. In some embodiments, the disorder may be associated with a decreased level of C3b or a C3b-containing complex, a decreased level of an activity/response associated with C3b or a C3b-containing complex, or a decreased level of a product of an activity/response associated with C3b or a C3b-containing complex as compared to the control state.

In some embodiments, the disorder may be a disorder associated with any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46 or a product of an activity/response associated with any one or more of FH, FHL-1, FI, FHR1-FHR5, FB, FD, CR1 and/or CD46. In some embodiments, the disorder is a disorder in which any one or more of FH, FHL-1, FI, FHR1-FHR5, FB, FD, CR1 and/or CD46, an activity/response associated with any one or more of FH, FHL-1, FI, FHR1-FHR5, FB, FD, CR1 and/or CD46, or the product of said activity/response is pathologically implicated. In some embodiments, the disorder may be associated with a decreased level of any one or more of FH, FHL-1, FI, FHR1-FHR5, FB, FD, CR1 and/or CD46, a decreased level of an activity/response associated with any one or more of FH, FHL-1, FI, FHR1-FHR5, FB, FD, CR1 and/or CD46, or a decreased level of a product of an activity/response associated with any one or more of FH, FHL-1, FI, FHR1-FHR5, FB, FD, CR1 and/or CD46 as compared to a control state.

In some embodiments, the disorder may be associated with an increased level of any one or more of FHR1-FHR5, an increased level of an activity/response associated with any one or more of FHR1-FHR5, or an increased level of a product of an activity/response associated with any one or more of FHR1-FHR5 as compared to a control state, see e.g. Zhu et al., Kidney Int. 2018 Jul;94(1):150-158; Pouw et al., Front Immunol. 2018 Apr 24;9:848; both hereby incorporated by reference in their entirety. In some embodiments the disorder may be associated with an increased level of FHR4, an increased level of an activity/response associated with FHR4, or an increased level of a product of an activity/response associated with FHR4 as compared to a control state, see e.g. WO 2019/215330 and Cipriani et al., Nat Commun 11, 778 (2020), both hereby incorporated by reference in their entirety. The methods may comprise determining the systemic level of FHR4.

In some embodiments the disorder is associated with increased levels of any one or more of C3, C3b, C3 convertase and/or C3bBb as compared to a control state. In some embodiments the disorder is associated with decreased levels of any one or more of C3, C3b, C3 convertase and/or C3bBb as compared to a control state. In some embodiments, the disorder is associated with increased levels of iC3b as compared to a control state. In some embodiments, the disorder is associated with decreased levels of iC3b as compared to a control state. In some embodiments the disorder is associated with increased levels of any one or more of C3a, C3f, C3c, C3dg, C3d, and/or C3g as compared to a control state. In some embodiments the disorder is associated with decreased levels of any one or more of C3a, C3f, C3c, C3dg, C3d, and/or C3g as compared to a control state.

In some cases, methods provided herein are useful for determining whether a complement-related disorder is associated with over-activation of the complement system. In some cases, the methods are capable of determining whether elevated levels of a complement protein, e.g. those described herein, are contributing to complement over-activation and/or complement-related disorders, e.g. as compared to a control subject that does not have a complement-related disorder.

The disorder may be an ocular disorder. In some embodiments, a disease or condition to be assessed, diagnosed, treated or prevented as described herein is a complement-related ocular disease. In some embodiments, the disorder is macular degeneration. In some embodiments, the disorder may be selected from, i.e. is one or more of, age-related macular degeneration (AMD), choroidal neovascularisation (CNV), macular dystrophy, and diabetic maculopathy. As used herein, the term “AMD” includes early AMD, intermediate AMD, late/advanced AMD, geographic atrophy (‘dry’ (i.e. non-exudative) AMD), and ‘wet’ (i.e. exudative or neovascular) AMD, each of which may be a disorder in its own right that is detected, treated and/or prevented as described herein. In some embodiments the disease or condition to be treated or prevented is a combination of the diseases/conditions above, e.g. ‘dry’ and ‘wet’ AMD. In some embodiments the disease or condition to be treated or prevented is not ‘wet’ AMD or choroidal neovascularisation. AMD is commonly-defined as causing vision loss in subjects age 50 and older. In some embodiments a subject to be treated is age 50 or older, i.e. is at least 50 years old.

As used herein “early AMD” refers to a stage of AMD characterised by the presence of medium-sized drusen, commonly having a diameter of up to ˜200 μm, within Bruch's membrane adjacent to the RPE layer. Subjects with early AMD typically do not present with significant vision loss. As used herein “intermediate AMD” refers to a stage of AMD characterised by large drusen and/or pigment changes in the retina. Intermediate AMD may be accompanied by some vision loss. As used herein “late AMD” refers to a stage of AMD characterised by the presence of drusen and vision loss, e.g. severe central vision loss, due to damage to the macula. In all stages of AMD, ‘reticular pseudodrusen’ (RPD) or ‘reticular drusen’ (also referred to as subretinal drusenoid deposits (SDD)) may be present, referring to the accumulation of extracellular material in the subretinal space between the neurosensory retina and RPE. “Late AMD” encompasses ‘dry’ and ‘Wet’ AMD. In ‘dry’ AMD (also known as geographic atrophy), there is a gradual breakdown of the light-sensitive cells in the macula that convey visual information to the brain and of the supporting tissue beneath the macula. In ‘wet’ AMD (also known as choroidal neovascularization, neovascular and exudative AMD), abnormal blood vessels grow underneath and into the retina. These vessels can leak fluid and blood which can lead to swelling and damage of the macula and subsequent scar formation. The damage may be rapid and severe.

In some embodiments the disorder is early-onset macular degeneration (EOMD). As used herein “EOMD” refers to a phenotypically severe sub-type of macular degeneration that demonstrates a much earlier age of onset than classical AMD and results in many more years of substantial visual loss. Sufferers may show an early-onset drusen phenotype comprising uniform small, slightly raised, yellow subretinal nodules randomly scattered in the macular, also known as ‘basal laminar drusen’ or ‘cuticular drusen’. EOMD may also be referred to as “middle-onset macular degeneration”. The EOMD subset is described in e.g. Boon C J et al. Am J Hum Genet 2008; 82(2):516-23, van de Ven J P, et al. Arch Ophthalmol 2012;130(8):1038-47, and Taylor, R. L. et al., Ophthalmol. 2019, 126, 1410-1421, all of which are hereby incorporated by reference in their entirety. As with other types of macular degeneration, EOMD is related to complement dysregulation and disrupted Factor H activity. In some embodiments a subject to be treated is age 49 or younger. In some embodiments a subject to be treated is between ages 15 and 49, i.e. is between 15 and 49 years old. In some embodiments the disease or condition to be treated is a macular dystrophy. A macular dystrophy can be a genetic condition, usually caused by a mutation in a single gene, that results in degeneration of the macula.

In some embodiments the disorder is one associated with the kidney, e.g. nephropathy/a nephropathic disorder. In some cases, the disorder is a neurological and/or neurodegenerative disorder. In some cases, the disorder is associated with autoimmunity, e.g. an autoimmune disease. In some cases, the disorder is associated with inflammation, e.g. an inflammatory disease. In some cases the disorder is characterised by the deposition of C3, e.g. the glomerular pathologies (see e.g. Skerka et al 2013, supra).

In some embodiments the disorder may be selected from Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), glomerular diseases, C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, multiple sclerosis (MS), stroke, Parkinson's disease, and Alzheimer's disease.

In some cases, the disorder is cancer. The cancer may be a liquid or blood cancer, such as leukemia, lymphoma or myeloma. In other cases, the cancer is a solid cancer, such as breast cancer, lung cancer, liver cancer, colorectal cancer, nasopharyngeal cancer, kidney cancer or glioma. In some cases, the cancer is located in the liver, bone marrow, lung, spleen, brain, pancreas, stomach or intestine. In some cases the cancer is lung cancer. In some cases the cancer is glioblastoma (glioblastoma multiforme (GBM)).

In some cases the disorder is neurodegeneration or neurodegenerative disease. The disorder may comprise progressive atrophy and loss of function of neurons. The disorder may be selected from Parkinson's disease, Alzheimer's disease, dementia, stroke, Lewy body disease, Amyotrophic lateral sclerosis (ALS), multiple sclerosis (MS), Huntington's disease and prion diseases.

The role of complement in various diseases is described in e.g. Morgan, B. P., Complement in the pathogenesis of Alzheimer's disease. Semin Immunopathol, 2018. 40(1): p. 113-124; Halbgebauer, R., et al., Janus face of complement-driven neutrophil activation during sepsis. Semin Immunol, 2018. 37: p. 12-20; Ma, Y., et al., Significance of Complement System in Ischemic Stroke: A Comprehensive Review. Aging Dis, 2019. 10(2): p. 429-462; Bonifati and Kishore, Role of complement in neurodegeneration and neuroinflammation. Mol Immunol. 2007 Feb;44(5):999-1010; Kleczko, E. K., et al., Targeting the Complement Pathway as a Therapeutic Strategy in Lung Cancer. Front Immunol, 2019. 10: p. 954; and Schafer N. et al., Complement Regulator FHR-3 Is Elevated either Locally or Systemically in a Selection of Autoimmune Diseases, Front Immunol. 2016; 7: 542, which are all hereby incorporated by reference in their entirety. For example, FHL-1 is expressed more in certain tumour cell lines than FH (Junnikkala et al (2000) J. Immunol. 164: 6075-81) and glioblastoma tumours have been shown to express FHR proteins (DeCordova et al. (2019) Immunobiology 224: 625-631), both references hereby incorporated in their entirety. Being able to measure and differentiate between FH family proteins is advantageous.

In some aspects, the present invention provides methods of predicting, based on the analysis described herein of a sample from a subject, whether a subject is at risk of developing a complement-related disorder, has a complement-related disorder, is in need of treatment for a complement-related disorder, will respond to treatment for a complement-related disorder, and/or is responding/has responded to treatment for a complement-related disorder. The methods may be used for determining whether a subject is at risk of onset of the disorder, and/or is at risk of progression, exacerbation or worsening of the disorder.

In one aspect, the present invention provides a method for determining whether a subject is at risk of developing a complement-related disorder, the method comprising detecting/determining the level of at least one complement protein, e.g. in a sample from the subject, digesting the protein(s) with endoproteinase GluC to obtain one or more peptides; and detecting the one or more peptides by mass spectrometry. The method may comprise an initial step of obtaining a sample and/or at least one protein from the subject. Suitable sources of samples are described herein. The method may include using the results of the mass spectrometry step to determine the likelihood of the subject to develop a complement-related disorder.

In one aspect, provided is the use of endoproteinase GluC in a method for determining the presence and/or level of a complement protein, e.g. in a sample or a subject, e.g. according to the methods described herein. Also provided is the use of endoproteinase GluC in a method of identifying a subject having a complement-related disorder or at risk of developing a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject has or is likely to develop a complement-related disorder.

Also provided is the use of GluC in a method of selecting a subject for treatment of a complement-related disorder with a complement-targeted therapeutic, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject is in need of a complement-targeted therapeutic.

The methods described herein may be used for determining whether a subject is at risk of onset of macular degeneration, e.g. EOMD and/or AMD, and/or is at risk of EOMD and/or AMD progression. In some cases, the disorder is selected from EOMD, AMD, geographic atrophy (‘dry’ (i.e. non-exudative) AMD), early AMD, intermediate AMD, late/advanced AMD, ‘wet’ (neovascular or exudative) AMD, choroidal neovascularisation (CNV) and retinal dystrophy. In some cases, the subject has or is suspected to have a complement-related disorder. In some cases the disorder is AMD. In some cases the disorder is EOMD.

Thus the present invention also provides a method for determining whether a subject is at risk of developing macular degeneration, e.g. EOMD and/or AMD, the method comprising detecting/determining the level of at least one complement protein, e.g. in a sample from the subject, digesting the protein(s) with endoproteinase GluC to obtain one or more peptides; and detecting the one or more peptides by mass spectrometry. The method may comprise an initial step of obtaining a sample and/or at least one protein from the subject. The method may include using the results of the mass spectrometry step to determine the likelihood of the subject to develop macular degeneration.

Also provided herein is a method for assessing the propensity or predisposition of a subject to develop a complement-related disorder, comprising:
(a) providing a blood sample from the subject;
(b) assessing the presence or level of at least one complement protein in the sample, as described herein;
(c) using the results of (b) to determine the likelihood of the subject to develop a complement-related disorder.

In other aspects, the present invention provides a method for identifying a subject at risk of developing, or having, a complement-related disorder, the method comprising detecting/determining the level of a complement protein as described herein. The disorder may be EOMD and/or AMD, or related disorders e.g. as described herein.

The present invention provides a method of identifying a subject having a complement-related disorder, the method comprising:
a) digesting at least one complement protein in a sample obtained from the subject with endoproteinase GluC to obtain one or more peptides;
b) determining the presence and/or level of the one or more peptides by mass spectrometry; and
c) using the results of (b) to determine whether the subject has a complement-related disorder.

Methods described herein may also be useful for assessing whether treatment for a complement-related disorder is/has been effective or successful.

In some aspects, the present invention provides methods employing the techniques described herein for determining whether a subject is likely to respond or not respond to a therapeutic treatment, or whether a subject is responding to a therapeutic treatment. Such methods should enable patients to receive the most effective therapy for their particular pathological requirements.

In some cases, an increase/decrease of a complement protein, e.g. as described herein, as compared to a reference value indicates an increased risk of developing a complement-related disorder. In some cases, an increase/decrease of a complement protein, e.g. as described herein, indicates an increased risk of developing the disorder as compared to a reference value taken from the same subject/a sample from the same subject at an earlier stage of the disorder.

In some embodiments, a method described herein may comprise determining the level of two or more complement proteins and comparing their values e.g. concentrations. The values may be compared to each other, as well as to reference values, e.g. increased levels of C3 and C3b compared to stationary or decreased levels of iC3b and further C3b breakdown products may be indicative of a higher risk of development of a complement-related disorder and/or the need to treat a subject for a complement-related disorder. Decreased levels of C3 and C3b compared to stationary or increased levels of iC3b and further C3b breakdown products may be indicative of a lower risk of development of a complement-related disorder and/or that treatment for a complement-related disorder is effective.

In some embodiments, a method provided herein comprises a step of correlating the presence of an atypical amount/level of a complement protein with an increased risk of the subject developing or having a complement-related disorder.

As used herein the term “reference value” refers to a known measurement value used for comparison during analysis. In some cases, the reference value is one or a set of test values obtained from an individual or group in a defined state of health. In some cases, the reference value is/has been obtained from determining the level of complement proteins in subjects known not to have a complement-related disorder. In some cases, the reference value is set by determining the level or amount of a complement protein previously from the same subject e.g. at an earlier stage of disease progression. The reference value may be taken from a sample obtained from the same subject, or a different subject or subject(s). The sample may be derived from the same tissue/cells/bodily fluid as the sample used by the present invention. The reference value may be a standard value, standard curve or standard data set. Values/levels which deviate significantly from reference values may be described as atypical values/levels.

In some cases the control may be a reference sample or reference dataset, or one or more values from said sample or dataset. The reference value may be derived from a reference sample or reference dataset. The reference value may be derived from one or more samples that have previously been obtained from one or more subjects that are known not to have a complement-related disorder and/or known or expected not to be at risk of developing a complement-related disorder. The reference value may be derived from one or more samples that have previously been obtained from one or more subjects that are known to have a complement-related disorder. The reference value may be derived from one or more samples that have previously been obtained from one or more subjects that are known to be at risk of developing a complement-related disorder. The reference value may be consensus level or an average, or mean, value calculated from a reference dataset, e.g. a mean protein level. The reference dataset/value may be obtained from a large-scale study of subjects known to have a complement-related disorder, such as AMD.

Examples of reference values for complement proteins in human subjects known not to have a complement-related disorder include:
a) FH: ˜150 to 500 μg/ml in human blood (Clark et al., J Immunol 2014. 193(10):4962-70 and unpublished data);
b) FHL-1: ˜0.5 to 50 μg/ml in human blood (Clark et al., J Immunol 2014. 193(10):4962-70 and unpublished data);
c) FHR1: ˜70 to 100 μg/ml in human plasma (Heinen, S et al., Blood 114, 2439-2447);
d) FHR2: ˜15-50 μg/ml in human plasma, or about 1/10 of FH concentration (Skerka et al., Mol Immunol 2013, 56:170-180);
e) FHR3: ˜70 to 100 μg/ml in human plasma (Fritsche, L. G. et al., Hum. Mol. Genet. 2010.19, 4694-4704);
f) FHR4: ≤5 μg/ml in human blood (WO 2019/215330);
g) FHR5: ˜1.5 μg/ml in human plasma (van Beek, A E et al., Front Immunol. 2017 Oct 18;8:1328);
h) FI: ˜35 μg/ml in human plasma;
i) C3: ˜0.5-16 mg/ml in human plasma (Engström, G. et al., J Hum Hypertens. 2007 Apr;21(4):276-82; Lee S H et al., Am J Respir Crit Care Med. 2006 Feb 15;173(4):370-8);
j) C3a: 46-157 ng/ml (Lee S H et al., Am J Respir Crit Care Med. 2006 Feb 15;173(4):370-8);
k) iC3b: ˜0.7-5 μg/ml (Kim A H J et al., Arthritis Rheumatol. 2019 Mar;71(3):420-430).

In some cases, mean reference values for circulating FH, FHL-1 and FHR1-5 in human subjects known not to have a complement-related disorder, e.g. AMD, include the following (95% Cl in parentheses):
a) FH, nM: 737.3 (718.2-756.5)
b) FHL-1, nM: 10.4 (10.1-10.8)
c) FHR-1, nM: 31.2 (29.4-32.9)
d) FHR-2, nM: 45.3 (43.1-47.6)
e) FHR-3, nM: 24.1 (21.7-26.5)
f) FHR-4, nM: 46.1 (42.7-49.6)
g) FHR-5, nM: 25.5 (24.5-26.5).

The relative concentrations of one complement protein to another can be determined using their reference values. For example, the ratio of the level of one complement protein to the level of another, or others, can be inferred from the concentrations provided above, e.g. FH:FHL-1, C3:iC3b, C3:C3b etc. The relative concentrations and/or ratios of the level of one complement protein to another, or others, may be altered in complement-related disorders. In some embodiments the methods provided herein involve detecting two or more complement proteins and determining how the levels of the complement proteins change with respect to one another as compared to a reference value(s). For example, the level of a first complement protein may increase as compared to the level of a second complement protein, or vice versa, e.g. FH vs FHL-1, C3 vs iC3b, C3 vs C3b.

Methods provided herein for assessing the risk of development, i.e. the onset or risk of progression of, or for identifying subjects having/at risk of, a complement-related disorder may be performed in conjunction with additional diagnostic methods and/or tests for such disorders that will be known to one skilled in the art. In some cases, methods for assessing the risk of development of a complement-related disorder comprise further techniques selected from: CH50 or AH50 measurement via haemolytic assay, measurement of neoantigen formation during MAC complex (C5b, C6, C7, C8, C9) generation, C3 deficiency screening, mannose-binding lectin assays, immunochemical assays to quantify individual complement components, flow cytometry to assess cell-bound regulatory proteins e.g. CD55, CD59 and CD35, and/or renal function tests, see e.g. Shih A R and Murali M R, Am. J. Hematol. 2015, 90(12):1180-1186, Ogedegbe H O, Laboratory Medicine, 2007, 38(5):295-304, and Gowda S et al., N Am J Med Sci. 2010, 2(4): 170-173, which are herein incorporated by reference in their entirety.

In some cases, methods provided herein for assessing the risk of development of AMD and/or EOMD comprise further assessment techniques selected from: dark adaptation testing, contrast sensitivity testing e.g. Pelli Robson, visual acuity testing using e.g. a Snellen chart and/or Amsler grid, Farnsworth-Munsell 100 hue test and Maximum Color Contrast Sensitivity test (MCCS) for assessing colour acuity and colour contrast sensitivity, preferential hyperacuity perimetry (PHP), fundus photography of the back of the eye, fundus examination, fundus autofluorescence, optical coherence tomography, angiography e.g. fluorescence angiography, fundus fluorescein angiography, indocyanine green angiography, optical coherence tomography angiography, adaptive optics retinal imaging, deep learning analysis of fundus images, electroretinogram methods, and/or methods to measure histological changes such as atrophy, retinal pigment changes, exudative changes e.g. hemorrhages in the eye, hard exudates, subretinal/sub-RPE/intraretinal fluid, and/or the presence of drusen.

### Treating Complement-Related Disorders

In some aspects, the methods of the present invention include treating a subject who is at risk of developing, predicted to be at risk of developing, who has been determined to be at risk of developing, or who has, has been identified as having, has been determined to have, or has been diagnosed as having a complement-related disorder, e.g. as described herein.

Any method provided herein for determining whether a subject is at risk of developing a complement-related disorder may additionally comprise a treatment step to treat said disorder. For example, a method provided herein for determining whether a subject is at risk of developing a complement-related disorder may comprise a treatment step to treat or prevent said disorder, wherein the subject has been determined to have atypical presence or levels of one or more complement proteins, e.g. detected/determined as described herein, as compared to a reference value(s).

A treatment step may comprise administering to a subject a therapeutically or prophylactically effective amount of one or more complement-targeted therapeutics, for example, one or more C1 inhibitors, C5 inhibitors, C5a inhibitors, C5aR antagonists, C3 inhibitors, C3a inhibitors, C3b inhibitors, C3aR antagonists, classical pathway inhibitors, alternative pathway inhibitors, FH-supplementation therapy and/or MBL pathway inhibitors. Specific complement-targeted therapeutics include without limitation one or more of human C1 esterase inhibitor (C1-INH), eculizumab (Soliris®, Alexion; a humanized monoclonal IgG2/4-antibody targeting C5), APL-2 (Apellis), mubodina (Adienne Pharma and Biotech), ergidina (Adienne Pharma and Biotech), POT-4 (a cyclic peptide inhibitor of C3; Alcon), rituximab (Biogen Idec, Genentech/Roche), ofatumumab (Genmab, GSK), compstatin analogues, soluble and targeted forms of CD59, PMX53 and PMX205, (Cephalon/Teva), JPE-1375 (Jerini), CCX168 (ChemoCentryx), NGD-2000-1 (former Neurogen), Cinryze (Shire), Berinert (CSL Behring), Cetor (Sanquin), Ruconest/Conestat alfa (Pharming), TNT009 (True North), OMS721 (Omeros), CLG561 (Novartis), AMY-101 (Amyndas), APL-1 (Apellis), APL-2 (Apellis), Mirococept (MRC), Lampalizumab (FCD4514S, Genentech/Roche), ACH-4471 (Achillion), ALXN1210 (Alexion), Tesidolumab/LFG316 (Novartis/Morphosys), Coversin (Akari), RA101495 (Ra Pharma), Zimura (ARC1905, Ophthotech), ALN-CC5 (Alnylam), IFX-1 (InflaRx), ALXN1007 (Alexion), Avacopan/CCX168 (Chemocentryx) and/or one or more therapeutic agents as described in e.g. Ricklin et al., Mol Immunol. 2017, 89:10-21; Ricklin and Lambris, Adv Exp Med Biol. 2013, 734: 1-22; Ricklin and Lambris, Semin Immunol. 2016, 28(3):208-22; Melis J P M et al., Mol Immunol. 2015 67(2):117-130; Thurman J M, Nephrol Dial Transplant, 2017 32: i57-i64, Cashman S M et al., PLoS One. 2011, 6(4):e19078; Bora N S et al., J Biol Chem. 2010, 285(44):33826-33; and Clark et al., J Clin Med 2015, 4(1):18-31 which are herein incorporated by reference in their entirety.

In some cases a treatment step comprises administering to a subject a therapeutically or prophylactically effective amount of one or more complement-targeted therapeutics described in WO 2018/224663 and/or WO 2019/138137, both hereby incorporated by reference in their entirety.

In some cases a complement-targeted therapeutic for use in the methods provided herein comprises a polypeptide which is capable of binding C3b, e.g. comprising an amino acid sequence having at least 85% identity to SEQ ID NO:145, 146, 147 or 148 and wherein the polypeptide has a total length of 450 amino acids or fewer, as described in WO 2019/138137. SEQ ID NO:145 to 148 described herein correspond to SEQ ID NO:4, 2, 3 and 13, respectively, described in WO 2019/138137. In some cases the complement-targeted therapeutic may have one or more of the following properties: binds to C3b, binds to C3b in the region of C3b bound by Complement Receptor 1, acts as a cofactor for FI, enables FI-mediated inactivation of C3b, reduces the amount of C3b via FI, increases the amount of C3b breakdown products e.g. iC3b, C3dg, C3d, C3f, e.g. via FI, and/or diffuses through BrM.

In some cases a complement-targeted therapeutic for use in the methods provided herein comprises a polypeptide comprising a C3b binding region and a C3b inactivating region, e.g. as described in WO 2018/224663, e.g. wherein the C3b inactivating region comprises, or consists of, an amino acid sequence having at least 65% sequence identity to the amino acid sequence of SEQ ID NO:149 and/or wherein the C3b binding region comprises, or consists of, an amino acid sequence having at least 65% sequence identity to the amino acid sequence of SEQ ID NO:150, 151 or 152. SEQ ID NO:149 to 152 described herein correspond to SEQ ID NO:9, 11, 13 and 14, respectively, described in WO 2018/224663. The polypeptide may comprise a linker between the C3b binding region and the C3b inactivating region. In some cases the polypeptide comprises a sequence comprising or consisting of an amino acid sequence having at least 65% sequence identity to the amino acid sequence of SEQ ID NO:32, 33 or 34 disclosed in WO 2018/224663. In some cases the complement-targeted therapeutic may have one or more of the following properties: binds to C3b, binds to C3b in the region of C3b bound by a cofactor for FI, acts as a cofactor for FI, enables FI-mediated inactivation of C3b, reduces the amount of C3b via FI, increases the amount of C3b breakdown products e.g. iC3b, C3dg, C3d, C3f, e.g. via FI, and/or diffuses through BrM.

In some cases, the subject has or is suspected to have a complement-related disorder. In some cases the disorder is AMD. In some cases the disorder is EOMD.

In some aspects, the present invention provides a method for treating or preventing a complement-related disorder in a subject, the method comprising administering an effective amount of a complement-targeted therapeutic, wherein the subject to be treated has been determined to have atypical presence or levels of one or more complement proteins, e.g. detected/determined as described herein, as compared to a reference value(s). In some aspects the subject has been determined to be at risk of developing a complement-related disorder, and/or identified as having a complement-related disorder.

In other aspects, the present invention provides a complement-targeted therapeutic for use in a method of treating or preventing a complement-related disorder in a subject, wherein the subject has/has been determined to have atypical presence or levels of one or more complement proteins, e.g. detected/determined as described herein, as compared to a reference value(s). In some aspects the subject has been determined to be at risk of developing a complement-related disorder, and/or identified as having a complement-related disorder.

In some aspects, provided is the use of a complement-targeted therapeutic in the manufacture of a medicament for treating or preventing a complement-related disorder in a subject, wherein the subject has/has been determined to have atypical presence or levels of one or more complement proteins, e.g. detected/determined as described herein, as compared to a reference value(s). In some aspects the subject has been determined to be at risk of developing a complement-related disorder, and/or identified as having a complement-related disorder.

Also provided is a method of treating or preventing a complement-related disorder in a subject, or a complement-targeted therapeutic for use in a method of treating or preventing a complement-related disorder in a subject, the method comprising administering an effective amount of a complement-targeted therapeutic wherein the subject is selected for treatment if the subject has/has been determined to have atypical presence or levels of one or more complement proteins, e.g. detected/determined as described herein, as compared to a reference value(s). In some aspects the subject has been determined to be at risk of developing a complement-related disorder, and/or identified as having a complement-related disorder.

The present invention also provides a method for selecting a subject for treatment with a complement-targeted therapeutic, comprising determining the presence and/or level of at least one complement protein, e.g. by detection/determining methods provided herein. The subject may have, or have been determined to have, a complement-related disorder, e.g. by methods provided herein.

In various aspects provided herein, the subject to be treated has atypical presence or levels of at least one complement protein, preferably one or more of FH, FHL-1, FHR1, FHR2, FHR3, FHR4, FHR5, FI, C3, C3b, C3a, iC3b, C3f, C3c, C3dg, and/or C3d. The subject may benefit from treatment to reduce the level of any complement proteins that are increased as compared to a reference value(s) and/or increase the level of any complement proteins that are decreased as compared to a reference value(s).

Methods described herein may comprise determining the level of one or more of FH, FHL-1, FHR1, FHR2, FHR3, FHR4, FHR5, FI, C3, C3b, C3a, iC3b, C3f, C3c, C3dg, and/or C3d and determining that the subject has or is likely to develop a complement-related disorder if the level of the measured protein(s) is altered, e.g. elevated or reduced, as compared to the level of that complement protein(s) in blood of a control subject that does not have a complement-related disorder, or the level of that complement protein(s) previously in blood of the subject of interest.

As used herein the term “reference value” refers to a known measurement value used for comparison during analysis. In some cases, the reference value is one or a set of test values obtained from an individual or group in a defined state of health. In some cases, the reference value is/has been obtained from determining the level of complement proteins in subjects known not to have a complement-related disorder. In some cases, the reference value is set by determining the level or amount of a complement protein previously from the same subject e.g. at an earlier stage of disease progression. The reference value may be taken from a sample obtained from the same subject, or a different subject or subject(s). The sample may be derived from the same tissue/cells/bodily fluid as the sample used by the present invention. The reference value may be a standard value, standard curve or standard data set. Values/levels which deviate significantly from reference values may be described as atypical values/levels.

In some cases, the methods described herein find use in diagnosing, treating or preventing, or selecting a subject for treatment or prevention of, a disorder which would benefit from one or more of: a reduction in the level or activity of one or more of C3bBb-type C3 convertase, C3bBb3b-type C5 convertase and/or C4b2a3b-type C5 convertase; a reduction in the level of one or more of C3, C3b, C3a, iC3b, FHR1, FHR2, FHR3, FHR4, FHR5, C5b and/or C5a; or an increase in the level of one or more of iC3b, C3f, C3c, C3dg, C3d, C3g, FH, FHL-1, FI, FH, FHL-1, FHR1, FHR2, FHR3, FHR4 and/or FHR5 as compared to reference value(s).

As used herein, ‘treatment’ may, for example, be reduction in the development or progression of a disease/condition, alleviation of the symptoms of a disease/condition or reduction in the pathology of a disease/condition. Treatment or alleviation of a disease/condition may be effective to prevent progression of the disease/condition, e.g. to prevent worsening of the condition or to slow the rate of development. In some embodiments treatment or alleviation may lead to an improvement in the disease/condition, e.g. a reduction in the symptoms of the disease/condition or reduction in some other correlate of the severity/activity of the disease/condition. Prevention/prophylaxis of a disease/condition may refer to prevention of a worsening of the condition or prevention of the development of the disease/condition, e.g. preventing an early stage disease/condition developing to a later, chronic, stage.

The methods provided herein may comprise determining in a subject the presence or absence of a genetic profile characterised by polymorphisms in the subject's genome associated with complement dysregulation. The polymorphisms may be found within or near genes such as CCL28, FBN2, ADAM12, PTPRC, IGLC1, HS3ST4, PRELP, PPID, SPOCK, APOB, SLC2A2, COL4A1, MYOC, ADAM19, FGFR2, C8A, FCN1, IFNAR2, C1NH, C7 and ITGA4. A genetic profile associated with complement dysregulation may comprise one or more, often multiple, single nucleotide polymorphisms, e.g. as set out in Tables I and II of US 2010/0303832, which is hereby incorporated by reference in its entirety.

Genetic factors are thought to play a role in the development of AMD and EOMD. Thus, any of the assessment or therapeutic methods described herein may be performed in conjunction with methods to assess AMD-associated and/or EOMD-associated and/or macular dystrophy-associated genetic variants. In some cases a complement-related disorder described herein may comprise a genetic element and/or a genetic risk factor.

In some cases, the methods provided herein further comprise determining in a subject the presence or absence of one or more genetic factors associated with AMD, e.g. one or more AMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors.

In some embodiments, the one or more genetic factors may be located on chromosome 1 at or near the RCA locus, e.g. in the CFH/CFHR genes.

The one or more genetic factors may be located in one or more of: CFH e.g. selected from Y402H (i.e. rs1061170^{C}), rs1410996^{C}, I62V (rs800292), A473A (rs2274700), R53C, D90G, D936E (rs1065489), R1210C, IVS1 (rs529825), IVS2 insTT, IVS6 (rs3766404), A307A (rs1061147), IVS10 (rs203674), rs3753396, R1210C, rs148553336, rs191281603, rs35292876, and rs800292; CFHR4 e.g. selected from rs6685931, and rs1409153; CFI e.g. selected from G119R, and rs141853578; CFB e.g. rs4151667, C2 e.g. rs9332739, C9 e.g. P167S; and/or C3 e.g. K155Q. In some embodiments, a genetic factor is Y402H (i.e. rs1061170^{C}). In some embodiments, a genetic factor is rs3753396. In some embodiments, a genetic factor is rs6685931 and/or rs1409153. In some embodiments, a genetic factor is not rs6685931.

Suitable genetic risk factors and genetic variants will be known in the art and may be as described in e.g. Edwards A O et al., Science 2005, 308(5720):421-4; Hageman G S et al., Proc Natl Aced Sci U S A. 2005, 102(20):7227-7232; Haines J L et al., Science 2005, 308(5720):419-21, Klein R J et al., Science 2005, 308(5720):385-389; Fritsche et al., Nat Genet. 2016, 48(2):134-43; US 2010/0303832; Clark et al., J Clin Med. 2015, 4(1):18-31; Cipriani, V. et al., Nat Commun. 2020, 11, 778; and Hageman G S et al, Hum Genomics. 2011, 5, 420 (2011), each hereby incorporated by reference in its entirety.

In some cases, the methods provided herein further comprise determining in a subject the presence or absence of one or more genetic factors associated with EOMD, e.g. one or more EOMD-associated genetic variants. In some cases, the methods comprise screening (directly or indirectly) for the presence or absence of the one or more genetic factors. In some embodiments, the genetic factor(s) are genetic risk factor(s). In some embodiments, the subject has been determined to have one or more such risk factors. In some embodiments, the methods of the present invention involve determining whether a subject possesses one or more such risk factors. In some embodiments the subject may possess one or more risk factors for early-onset macular degeneration (EOMD).

EOMD is thought to be caused by monogenic inheritance of rare variants of the CFH gene (see e.g. Boon C J et al. Am J Hum Genet 2008; 82(2):516-23; van de Ven J P, et al. Arch Ophthalmol 2012;130(8):1038-47; Yu Yet al. Hum Mol Genet 2014; 23(19):5283-93; Duvvari M R, et al. Mol Vis 2015; 21:285-92; Hughes A E, et al. Acta Ophthalmol 2016; 94(3):e247-8; Wagner et al. Sci Rep 2016;6:31531; Taylor R L et al, Ophthalmology. 2019 Mar 21. pii: S0161-6420(18):33171-3). In some embodiments, the subject may possess one or more of EOMD-associated genetic variants. EOMD-associated genetic variants are described in e.g. Servais A et al. Kidney Int, 2012; 82(4):454-64 and Dragon-Durey M A, et al. J Am Soc Nephrol 2004; 15(3):787-95; which are hereby incorporated by reference in their entirety. In some embodiments, the subject may possess one or more of the following EOMD-associated genetic variants: CFH c.1243del, p.(Ala415Profs*39) het; CFH c.350+1G>T het; CR-I c.619+1G>A het; CFH c.380G>A, p.(Arg127His); CFH c.694C>T, p.(Arg232Ter); or CFH c.1291T>A, p.(Cys431Ser).

In some cases, the methods provided herein comprise screening for deletions within the RCA locus (a region of DNA sequence located on chromosome one that extends from the CFH gene through to the CD46 (MCP) gene) that are associated with AMD risk or protection.

Methods for determining the presence or absence of genetic factors include restriction fragment length polymorphism identification (RFLPI) of genomic DNA, random amplified polymorphic detection (RAPD) of genomic DNA, amplified fragment length polymorphism detection (AFLPD), multiple locus variable number tandem repeat (VNTR) analysis (MLVA), SNP genotyping, multilocus sequence typing, PCR, DNA sequencing e.g. Sanger sequencing or Next-Generation sequencing, allele specific oligonucleotide (ASO) probes, and oligonucleotide microarrays or beads. Other suitable methods are described in e.g. Edenberg H J and Liu Y, Cold Spring Harb Protoc; 2009; doi:10.1101/pdb.top62, and Tsuchihashi Z and Dracopoli N C, Pharmacogenomics J., 2002, 2:103-110.

In some embodiments, the subject is selected for therapeutic or prophylactic treatment with a complement-targeted therapeutic based on their being determined to possess one or more genetic factors for AMD and/or EOMD, e.g. one or more AMD-associated and/or EOMD-associated genetic variants or a macular dystrophy. In some embodiments, the subject has been determined to have one or more such genetic factors. In some embodiments, the methods provided herein comprise determining whether a subject possesses one or more such genetic factors. Such methods and genetic factors are described herein. Thus, provided herein is a method of diagnosing, treating or preventing a complement-related disorder in a subject, wherein the subject has/has been determined/is determined to possess one or more genetic factors for AMD and/or EOMD, and wherein the subject has/has been determined/is determined to have atypical presence or levels of one or more complement proteins, e.g. detected/determined as described herein, as compared to a reference value(s); optionally wherein the method comprises administering a complement-targeted therapy/therapeutic agent.

The term “subject” refers to a subject, patient or individual and may be any animal or human. The subject is preferably mammalian, more preferably human. The subject may be a non-human mammal, but is more preferably human. The subject may be male or female. The subject may be a patient. Therapeutic uses may be in human or animals (veterinary use). The subject to be treated with a therapeutic substance described herein may be a subject in need thereof.

A subject described herein may belong to a patient subpopulation i.e. the subject may be part of an identifiable, specific portion or subdivision of a population. The population and/or subpopulation may have or be suspected to have a complement-related disorder. The subpopulation may display atypical presence or levels of one or more complement proteins, e.g. detected/determined as described herein, as compared to the population as a whole. The population and/or subpopulation may have or be suspected to have AMD, EOMD or a macular dystrophy.

The subject may be identified, or may have been identified, as having a complement-related disorder or being at risk of developing a complement-related disorder, e.g. by a method described herein.

In some aspects provided herein, the subject is characterised as having an atypical presence or level of one or more complement proteins, e.g. detected/determined/measured as described herein.

Provided is a method of treating or preventing a complement-related disorder in a subject, wherein is characterised as having an atypical presence or levels of one or more complement proteins, e.g. detected/determined as described herein.

Also provided is a complement-targeted therapeutic for use in a method of treating or preventing a complement-related disease in a subject, wherein the subject is characterised as having an atypical presence or levels of one or more complement proteins, e.g. detected/determined as described herein.

Methods according to the present invention may be performed outside the human or animal body. Methods according to the present invention may be performed, or products may be present, in vitro, ex vivo, or in vivo. The term “in vitro” is intended to encompass experiments with materials, biological substances, cells and/or tissues in laboratory conditions or in culture whereas the term “in vivo” is intended to encompass experiments and procedures with intact multi-cellular organisms. “Ex vivo” refers to something present or taking place outside an organism, e.g. outside the human or animal body, which may be on tissue (e.g. whole organs) or cells taken from the organism. In some embodiments, the determining, detecting, measuring, quantifying, predicting and/or diagnosing steps of the methods provided herein are performed in vitro.

Complement-targeted therapeutics described herein may be administered, or formulated for administration, by a number of routes, including but not limited to systemic, intratumoral, intraperitoneal, parenteral, intravenous, intra-arterial, intradermal, subcutaneous, intramuscular, oral and nasal. Preferably, the therapeutics are administered by a route selected from intratumoral, intraperitoneal or intravenous. The medicaments and compositions may be formulated in fluid or solid form. Fluid formulations may be formulated for administration by injection to a selected region of the human or animal body.

Administration is preferably in a “therapeutically effective amount”, this being sufficient to show benefit to the individual. The actual amount administered, and rate and time-course of administration, will depend on the nature and severity of the disease being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, Edition, 2000, pub. Lippincott, Williams & Wilkins.

### Kits

Aspects of the disclosure include in vitro diagnostic methods and in vitro kits for performing such methods. In some embodiments, the present invention provides a kit comprising endoproteinase GluC for use in a method of detecting and/or determining the level of one or more complement protein(s) e.g. in a sample. The kit may be used for any of the methods described herein and/or for detecting/determining the level of any one or combination of proteins described herein. The kit may be suitable for, used for, or intended/sold/distributed for detecting at least one complement protein in a sample, determining the level of at least one complement protein in a sample, preparing at least one complement protein for analysis and/or detection, determining the presence and/or level of a complement protein in a subject, determining whether a subject is at risk of developing a complement-related disorder, identifying a subject having a complement-related disorder, selecting a patient/subject for treatment of a complement-related disorder, and/or treating a subject who is suspected to have a complement-related disorder. The kit and components thereof may be suitable for use with MS techniques.

A kit provided herein comprises one, two, or more components suitable for performing the methods described herein, in whole or in part. The kit may comprise standards or controls, e.g. labelled peptide standard(s) for each protein to be detected using the kit. The kit may comprise a predetermined quantity of labelled peptide standards. The kit may comprise a predetermined quantity of GluC enzyme, optionally with the necessary buffers and reagents for enzyme digestion. The components of the kit may be provided in a single composition, or may be provided as plural compositions.

The kit may be suitable for a point-of-care in vitro diagnostic test. It may be a kit for laboratory-based testing. The kit may include instructions for use, such as an instruction booklet or leaflet. The instructions may include a protocol for performing any one or more of the methods described herein e.g. for enzyme digestion, recommended MS settings, and/or data analysis templates. The kit may comprise components for separating proteins in a sample and/or performing MS techniques e.g. liquid chromatography columns.

The kit may be adapted for use with dry samples, wet samples, frozen samples, fixed samples, urine samples, saliva samples, tissue samples, blood samples, or any other type of sample, including any of the sample types disclosed herein. The kit may comprise a device for obtaining or processing a blood, serum, plasma, cell or tissue sample.

### Sequence Identity

As used herein, an amino acid sequence which corresponds to a reference amino acid sequence may comprise at least 60%, e.g. one of at least 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% sequence identity to the reference sequence.

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780 software. When using such software, the default parameters, e.g. for gap penalty and extension penalty, are preferably used.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof. The invention includes the combination of the aspects and preferred features described except where such a combination is clearly impermissible or expressly avoided.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

The phase “and/or” as used herein encompasses each member of the list individually, as well as any combination of one or members of the list up to and including every member of the list.

For standard molecular biology techniques, see Sambrook, J., Russel, D. W. Molecular Cloning, A Laboratory Manual. 3 ed. 2001, Cold Spring Harbor, New York: Cold Spring Harbor Laboratory Press.

### Sequences

| SEQ ID | | |
|---|---|---|
| No: | Description | Sequence |
| 1 | Human Complement | MRLLAKIICLMLWAICVAEDCNELPPRRNTEILTG |
| | Factor H (FH) | SWSDQTYPEGTQAIYKCRPGYRSLGNVIMVCRKGE |
| | Uniprot: P08603-1 | WVALNPLRKCQKRPCGHPGDTPFGTFTLTGGNVFE |
| | Entry version 229 | YGVKAVYTCNEGYQLLGEINYRECDTDGWTNDIPI |
| | (11 Dec. 2019) | CEVVKCLPVTAPENGKIVSSAMEPDREYHFGQAVR |
| | Sequence version 4 | FVCNSGYKIEGDEEMHCSDDGFWSKEKPKCVEISC |
| | (11 Sep. 2007) | KSPDVINGSPISQKIIYKENERFQYKCNMGYEYSE |
| | | RGDAVCTESGWRPLPSCEEKSCDNPYIPNGDYSPL |
| | | RIKHRTGDEITYQCRNGFYPATRGNTAKCTSTGWI |
| | | PAPRCTLKPCDYPDIKHGGLYHENMRRPYFPVAVG |
| | | KYYSYYCDEHFETPSGSYWDHIHCTQDGWSPAVPC |
| | | LRKCYFPYLENGYNQNYGRKFVQGKSIDVACHPGY |
| | | ALPKAQTTVTCMENGWSPTPRCIRVKTCSKSSIDI |
| | | ENGFISESQYTYALKEKAKYQCKLGYVTADGETSG |
| | | SITCGKDGWSAQPTCIKSCDIPVFMNARTKNDFTW |
| | | FKLNDTLDYECHDGYESNTGSTTGSIVCGYNGWSD |
| | | LPICYERECELPKIDVHLVPDRKKDQYKVGEVLKF |
| | | SCKPGFTIVGPNSVQCYHFGLSPDLPICKEQVQSC |
| | | GPPPELLNGNVKEKTKEEYGHSEVVEYYCNPRFLM |
| | | KGPNKIQCVDGEWTTLPVCIVEESTCGDIPELEHG |
| | | WAQLSSPPYYYGDSVEFNCSESFTMIGHRSITCIH |
| | | GVWTQLPQCVAIDKLKKCKSSNLIILEEHLKNKKE |
| | | FDHNSNIRYRCRGKEGWIHTVCINGRWDPEVNCSM |
| | | AQIQLCPPPPQIPNSHNMTTTLNYRDGEKVSVLCQ |
| | | ENYLIQEGEEITCKDGRWQSIPLCVEKIPCSQPPQ |
| | | IEHGTINSSRSSQESYAHGTKLSYTCEGGFRISEE |
| | | NETTCYMGKWSSPPQCEGLPCKSPPEISHGVVAHM |
| | | SDSYQYGEEVTYKCFEGFGIDGPAIAKCLGEKWSH |
| | | PPSCIKTDCLSLPSFENAIPMGEKKDVYKAGEQVT |
| | | YTCATYYKMDGASNVTCINSRWTGRPTCRDTSCVN |
| | | PPTVQNAYIVSRQMSKYPSGERVRYQCRSPYEMFG |
| | | DEEVMCLNGNWTEPPQCKDSTGKCGPPPPIDNGDI |
| | | TSFPLSVYAPASSVEYQCQNLYQLEGNKRITCRNG |
| | | QWSEPPKCLHPCVISREIMENYNIALRWTAKQKLY |
| | | SRTGESVEFVCKRGYRLSSRSHTLRTTCWDGKLEY |
| | | PTCAKR |
| 2 | Human FH-like | MRLLAKIICLMLWAICVAEDCNELPPRRNTEILTG |
| | protein 1 (FHL-1) | SWSDQTYPEGTQAIYKCRPGYRSLGNVIMVCRKGE |
| | Uniprot: P08603-2 | WVALNPLRKCQKRPCGHPGDTPFGTFTLTGGNVFE |
| | Entry version 229 | YGVKAVYTCNEGYQLLGEINYRECDTDGWTNDIPI |
| | (11 Dec. 2019) | CEVVKCLPVTAPENGKIVSSAMEPDREYHFGQAVR |
| | Sequence version 4 | FVCNSGYKIEGDEEMHCSDDGFWSKEKPKCVEISC |
| | (11 Sep. 2007) | KSPDVIN |
| | | GSPISQKIIYKENERFQYKCNMGYEYSERGDAVCT |
| | | ESGWRPLPSCEEKSCDNPYIPNGDYSPLRIKHRTG |
| | | DEITYQCRNGFYPATRGNTAKCTSTGWIPAPRCTL |
| | | KPCDYPDIKHGGLYHENMRRPYFPVAVGKYYSYYC |
| | | DEHFETPSGSYWDHIHCTQDGWSPAVPCLRKCYFP |
| | | YLENGYNQNYGRKFVQGKSIDVACHPGYALPKAQT |
| | | TVTCMENGWSPTPRCIRVSFTL |
| 3 | Complement Factor | MWLLVSVILISRISSVGGEATFCDFPKINHGILYD |
| | H-related 1 (FHR-1) | EEKYKPFSQVPTGEVFYYSCEYNFVSPSKSFWTRI |
| | Uniprot: Q03591 | TCTEEGWSPTPKCLRLCFFPFVENGHSESSGQTHL |
| | Entry version 166 | EGDTVQIICNTGYRLQNNENNISCVERGWSTPPKC |
| | (11 Dec. 2019) | RSTDTSCVNPPTVQNAHILSRQMSKYPSGERVRYE |
| | Sequence version 2 | CRSPYEMFGDEEVMCLNGNWTEPPQCKDSTGKCGP |
| | (16 Dec. 2008) | PPPIDNGDITSFPLSVYAPASSVEYQCQNLYQLEG |
| | | NKRITCRNGQWSEPPKCLHPCVISREIMENYNIAL |
| | | RWTAKQKLYLRTGESAEFVCKRGYRLSSRSHTLRT |
| | | TCWDGKLEYPTCAKR |
| 4 | Complement Factor | MWLLVSVILISRISSVGGEAMFCDFPKINHGILYD |
| | H-related 2 (FHR-2), | EEKYKPFSQVPTGEVFYYSCEYNFVSPSKSFWTRI |
| | isoform 1 | TCAEEGWSPTPKCLRLCFFPFVENGHSESSGQTHL |
| | Uniprot: P36980-1 | EGDTVQIICNTGYRLQNNENNISCVERGWSTPPKC |
| | Entry version 168 | RSTISAEKCGPPPPIDNGDITSFLLSVYAPGSSVE |
| | (11 Dec. 2019) | YQCQNLYQLEGNNQITCRNGQWSEPPKCLDPCVIS |
| | Sequence version 1 | QEIMEKYNIKLKWTNQQKLYSRTGDIVEFVCKSGY |
| | (01 Jun. 1994) | HPTKSHSFRAMCQNGKLVYPSCEEK |
| 5 | Complement Factor | MWLLVSVILISRISSVGGEAMFCDFPKINHGILYD |
| | H-related 2 (FHR-2), | EEKYKPFSQVPTGEVFYYSCEYNFVSPSKSFWTRI |
| | isoform 2 | TCAEEGWSPTPKCLRLCFFPFVENGHSESSGQTHL |
| | Uniprot: P36980-2 | EGDTVQIICNTGYRLQNNENNISCVERGWSTPPKC |
| | Entry version 168 | RSTSSSVEYQCQNLYQLEGNNQITCRNGQWSEPPK |
| | (11 Dec. 2019) | CLDPCVISQEIMEKYNIKLKWTNQQKLYSRTGDIV |
| | Sequence version 1 | EFVCKSGYHPTKSHSFRAMCQNGKLVYPSCEEK |
| | (01 Jun. 1994) | |
| 6 | Complement Factor | MLLLINVILTLWVSCANGQVKPCDFPDIKHGGLFH |
| | H-related 3 (FHR-3), | ENMRRPYFPVAVGKYYSYYCDEHFETPSGSYWDYI |
| | isoform 1 | HCTQNGWSPAVPCLRKCYFPYLENGYNQNYGRKFV |
| | Uniprot: Q02985-1 | QGNSTEVACHPGYGLPKAQTTVTCTEKGWSPTPRC |
| | Entry version 161 | IRVRTCSKSDIEIENGFISESSSIYILNKEIQYKC |
| | (11 Dec. 2019) | KPGYATADGNSSGSITCLQNGWSAQPICINSSEKC |
| | Sequence version 2 | GPPPPISNGDTTSFLLKVYVPQSRVEYQCQPYYEL |
| | (21 Feb. 2001) | QGSNYVTCSNGEWSEPPRCIHPCIITEENMNKNNI |
| | | KLKGRSDRKYYAKTGDTIEFMCKLGYNANTSILSF |
| | | QAVCREGIVEYPRCE |
| 7 | Complement Factor | MLLLINVILTLWVSCANGQVKPCDFPDIKHGGLFH |
| | H-related 3 (FHR-3), | ENMRRPYFPVAVGKYYSYYCDEHFETPSGSYWDYI |
| | isoform 2 | HCTQNGWSPAVPCLRKCYFPYLENGYNQNYGRKFV |
| | Uniprot: Q02985-2 | QGNSTEVACHPGYGLPKAQTTVTCTEKGWSPTPRC |
| | Entry version 161 | IRVNSSEKCGPPPPISNGDTTSFLLKVYVPQSRVE |
| | (11 Dec. 2019) | YQCQPYYELQGSNYVTCSNGEWSEPPRCIHPCIIT |
| | Sequence version 2 | EENMNKNNIKLKGRSDRKYYAKTGDTIEFMCKLGY |
| | (21 Feb. 2001) | NANTSILSFQAVCREGIVEYPRCE |
| 8 | Complement Factor | MLLLINVILTLWVSCANGQEVKPCDFPEIQHGGLY |
| | H-related 4 (FHR- | YKSLRRLYFPAAAGQSYSYYCDQNFVTPSGSYWDY |
| | 4A) | IHCTQDGWSPTVPCLRTCSKSDVEIENGFISESSS |
| | Uniprot: Q92496-1 | IYILNEETQYNCKPGYATAEGNSSGSITCLQNGWS |
| | Entry version 157 | TQPICIKFCDMPVFENSRAKSNGMWFKLHDTLDYE |
| | (11 Dec. 2019) | CYDGYESSYGNTTDSIVCGEDGWSHLPTCYNSSEN |
| | Sequence version 3 | CGPPPPISNGDTTSFPQKVYLPWSRVEYQCQSYYE |
| | (22 Jan. 2014) | LQGSKYVTCSNGDWSEPPRCISMKPCEFPEIQHGH |
| | | LYYENTRRPYFPVATGQSYSYYCDQNFVTPSGSYW |
| | | DYIHCTQDGWLPTVPCLRTCSKSDIEIENGFISES |
| | | SSIYILNKEIQYKCKPGYATADGNSSGSITCLQNG |
| | | WSAQPICIKFCDMPVFENSRAKSNGMRFKLHDTLD |
| | | YECYDGYEISYGNTTGSIVCGEDGWSHFPTCYNSS |
| | | EKCGPPPPISNGDTTSFLLKVYVPQSRVEYQCQSY |
| | | YELQGSNYVTCSNGEWSEPPRCIHPCIITEENMNK |
| | | NNIQLKGKSDIKYYAKTGDTIEFMCKLGYNANTSV |
| | | LSFQAVCREGIVEYPRCE |
| 9 | Complement Factor | MLLLINVILTLWVSCANGQEVKPCDFPEIQHGGLY |
| | H-related 4 (FHR- | YKSLRRLYFPAAAGQSYSYYCDQNFVTPSGSYWDY |
| | 4B) | IHCTQDGWSPTVPCLRTCSKSDIEIENGFISESSS |
| | Uniprot: Q92496-3 | IYILNKEIQYKCKPGYATADGNSSGSITCLQNGWS |
| | Entry version 157 | AQPICIKFCDMPVFENSRAKSNGMRFKLHDTLDYE |
| | (11 Dec. 2019) | CYDGYEISYGNTTGSIVCGEDGWSHFPTCYNSSEK |
| | Sequence version 3 | CGPPPPISNGDTTSFLLKVYVPQSRVEYQCQSYYE |
| | (22 Jan. 2014) | LQGSNYVTCSNGEWSEPPRCIHPCIITEENMNKNN |
| | | IQLKGKSDIKYYAKTGDTIEFMCKLGYNANTSVLS |
| | | FQAVCREGIVEYPRCE |
| 10 | Complement Factor | MLLLFSVILISWVSTVGGEGTLCDFPKIHHGFLYD |
| | H-related 5 (FHR-5) | EEDYNPFSQVPTGEVFYYSCEYNFVSPSKSFWTRI |
| | Uniprot: Q9BXR6 | TCTEEGWSPTPKCLRMCSFPFVKNGHSESSGLIHL |
| | | EGDTVQIICNTGYSLQNNEKNISCVERGWSTPPIC |
| | | SFTKGECHVPILEANVDAQPKKESYKVGDVLKFSC |
| | | RKNLIRVGSDSVQCYQFGWSPNFPTCKGQVRSCGP |
| | | PPQLSNGEVKEIRKEEYGHNEVVEYDCNPNFIING |
| | | PKKIQCVDGEWTTLPTCVEQVKTCGYIPELEYGYV |
| | | QPSVPPYQHGVSVEVNCRNEYAMIGNNMITCINGI |
| | | WTELPMCVATHQLKRCKIAGVNIKTLLKLSGKEFN |
| | | HNSRIRYRCSDIFRYRHSVCINGKWNPEVDCTEKR |
| | | EQFCPPPPQIPNAQNMTTTVNYQDGEKVAVLCKEN |
| | | YLLPEAKEIVCKDGRWQSLPRCVESTAYCGPPPSI |
| | | NNGDTTSFPLSVYPPGSTVTYRCQSFYKLQGSVTV |
| | | TCRNKQWSEPPRCLDPCVVSEENMNKNNIQLKWRN |
| | | DGKLYAKTGDAVEFQCKFPHKAMISSPPFRAICQE |
| | | GKFEYPICE |
| 11 | Complement Factor | MKLLHVFLLFLCFHLRFCKVTYTSQEDLVEKKCLA |
| | I (FI) | KKYTHLSCDKVFCQPWQRCIEGTCVCKLPYQCPKN |
| | | GTAVCATNRRSFPTYCQQKSLECLHPGTKFLNNGT |
| | | CTA |
| 12 | Uniprot: P05156 | EGKFSVSLKHGNTDSEGIVEVKLVDQDKTMFICKS |
| | Entry version 147 | SWSMREANVACLDLGFQQGADTQRRFKLSDLSINS |
| | (11 Dec. 2019) | TECLHVHCRGLETSLAECTFTKRRTMGYQDFADVV |
| | Sequence version 1 | CYTQKADSPMDDFFQCVNGKYISQMKACDGINDCG |
| | (01 Jun. 2001) | DQSDELCCKACQGKGFHCKSGVCIPSQYQCNGEVD |
| | Complement factor | CITGEDEVGCAGFASVTQEETEILTADMDAERRRI |
| | C3 | KSLLPKLSCGVKNRMHIRRKRIVGGKRAQLGDLPW |
| | (Uniprot: P01024) | QVAIKDASGITCGGIYIGGCWILTAAHCLRASKTH |
| | Entry version 242 | RYQIWTTVVDWIHPDLKRIVIEYVDRIIFHENYNA |
| | (11 Dec. 2019) | GTYQNDIALIEMKKDGNKKDCELPRSIPACVPWSP |
| | Sequence version 2 | YLFQPNDTCIVSGWGREKDNERVFSLQWGEVKLIS |
| | (12 Dec. 2006) | NCSKFYGNRFYEKEMECAGTYDGSIDACKGDSGGP |
| | | LVCMDANNVTYVWGVVSWGENCGKPEFPGVYTKVA |
| | | NYFDWISYHVGRPFISQYNVMGPTSGPSLLLLLLT |
| | | HLPLALGSPMYSIITPNILRLESEETMVLEAHDAQ |
| | | GDVPVTVTVHDFPGKKLVLSSEKTVLTPATNHMGN |
| | | VTFTIPANREFKSEKGRNKFVTVQATFGTQVVEKV |
| | | VLVSLQSGYLFIQTDKTIYTPGSTVLYRIFTVNHK |
| | | LLPVGRTVMVNIENPEGIPVKQDSLSSQNQLGVLP |
| | | LSWDIPELVNMGQWKIRAYYENSPQQVFSTEFEVK |
| | | EYVLPSFEVIVEPTEKFYYIYNEKGLEVTITARFL |
| | | YGKKVEGTAFVIFGIQDGEQRISLPESLKRIPIED |
| | | GSGEVVLSRKVLLDGVQNPRAEDLVGKSLYVSATV |
| | | ILHSGSDMVQAERSGIPIVTSPYQIHFTKTPKYFK |
| | | PGMPFDLMVFVTNPDGSPAYRVPVAVQGEDTVQSL |
| | | TQGDGVAKLSINTHPSQKPLSITVRTKKQELSEAE |
| | | QATRTMQALPYSTVGNSNNYLHLSVLRTELRPGET |
| | | LNVNFLLRMDRAHEAKIRYYTYLIMNKGRLLKAGR |
| | | QVREPGQDLVVLPLSITTDFIPSFRLVAYYTLIGA |
| | | SGQREVVADSVWVDVKDSCVGSLVVKSGQSEDRQP |
| | | VPGQQMTLKIEGDHGARVVLVAVDKGVFVLNKKNK |
| | | LTQSKIWDVVEKADIGCTPGSGKDYAGVFSDAGLT |
| | | FTSSSGQQTAQRAELQCPQPAARRRRSVQLTEKRM |
| | | DKVGKYPKELRKCCEDGMRENPMRFSCQRRTRFIS |
| | | LGEACKKVELDCCNYITELRRQHARASHLGLARSN |
| | | LDEDIIAEENIVSRSEFPESWLWNVEDLKEPPKNG |
| | | ISTKLMNIFLKDSITTWEILAVSMSDKKGICVADP |
| | | FEVTVMQDFFIDLRLPYSVVRNEQVEIRAVLYNYR |
| | | QNQELKVRVELLHNPAFCSLATTKRRHQQTVTIPP |
| | | KSSLSVPYVIVPLKTGLQEVEVKAAVYHHFISDGV |
| | | RKSLKVVPEGIRMNKTVAVRTLDPERLGREGVQKE |
| | | DIPPADLSDQVPDTESETRILLQGTPVAQMTEDAV |
| | | DAERLKHLIVTPSGCGEQNMIGMTPTVIAVHYLDE |
| | | TEQWEKFGLEKRQGALELIKKGYTQQLAFRQPSSA |
| | | FAAFVKRAPSTWLTAYVVKVFSLAVNLIAIDSQVL |
| | | CGAVKWLILEKQKPDGVFQEDAPVIHQEMIGGLRN |
| | | NNEKDMALTAFVLISLQEAKDICEEQVNSLPGSIT |
| | | KAGDFLEANYMNLQRSYTVAIAGYALAQMGRLKGP |
| | | LLNKFLTTAKDKNRWEDPGKQLYNVEATSYALLAL |
| | | LQLKDFDFVPPVVRWLNEQRYYGGGYGSTQATFMV |
| | | FQALAQYQKDAPDHQELNLDVSLQLPSRSSKITHR |
| | | IHWESASLLRSEETKENEGFTVTAEGKGQGTLSVV |
| | | TMYHAKAKDQLTCNKFDLKVTIKPAPETEKRPQDA |
| | | KNTMILEICTRYRGDQDATMSILDISMMTGFAPDT |
| | | DDLKQLANGVDRYISKYELDKAFSDRNTLIIYLDK |
| | | VSHSEDDCLAFKVHQYFNVELIQPGAVKVYAYYNL |
| | | EESCTRFYHPEKEDGKLNKLCRDELCRCAEENCFI |
| | | QKSDDKVTLEERLDKACEPGVDYVYKTRLVKVQLS |
| | | NDFDEYIMAIEQTIKSGSDEVQVGQQRTFISPIKC |
| | | REALKLEEKKHYLMWGLSSDFWGEKPNLSYIIGKD |
| | | TWVEHWPEEDECQDEENQKQCQDLGAFTESMVVFG |
| | | CPN |
| 13 | Human C3 β chain | SPMYSIITPNILRLESEETMVLEAHDAQGDVPVTV |
| | UniProt: P01024; | TVHDFPGKKLVLSSEKTVLTPATNHMGNVTFTIPA |
| | Entry version 242 | NREFKSEKGRNKFVTVQATFGTQVVEKVVLVSLQS |
| | (11 Dec. 2019) | GYLFIQTDKTIYTPGSTVLYRIFTVNHKLLPVGRT |
| | Sequence version 2 | VMVNIENPEGIPVKQDSLSSQNQLGVLPLSWDIPE |
| | (12 Dec. 2006); | LVNMGQWKIRAYYENSPQQVFSTEFEVKEYVLPSF |
| | residues 23-667 | EVIVEPTEKFYYIYNEKGLEVTITARFLYGKKVEG |
| | | TAFVIFGIQDGEQRISLPESLKRIPIEDGSGEVVL |
| | | SRKVLLDGVQNPRAEDLVGKSLYVSATVILHSGSD |
| | | MVQAERSGIPIVTSPYQIHFTKTPKYFKPGMPFDL |
| | | MVFVTNPDGSPAYRVPVAVQGEDTVQSLTQGDGVA |
| | | KLSINTHPSQKPLSITVRTKKQELSEAEQATRTMQ |
| | | ALPYSTVGNSNNYLHLSVLRTELRPGETLNVNFLL |
| | | RMDRAHEAKIRYYTYLIMNKGRLLKAGRQVREPGQ |
| | | DLVVLPLSITTDFIPSFRLVAYYTLIGASGQREVV |
| | | ADSVWVDVKDSCVGSLVVKSGQSEDRQPVPGQQMT |
| | | LKIEGDHGARVVLVAVDKGVFVLNKKNKLTQSKIW |
| | | DVVEKADIGCTPGSGKDYAGVFSDAGLTFTSSSGQ |
| | | QTAQRAELQCPQPAA |
| 14 | Human C3 α′ chain | SNLDEDIIAEENIVSRSEFPESWLWNVEDLKEPPK |
| | UniProt: P01024; | NGISTKLMNIFLKDSITTWEILAVSMSDKKGICVA |
| | Entry version 242 | DPFEVTVMQDFFIDLRLPYSVVRNEQVEIRAVLYN |
| | (11 Dec. 2019) | YRQNQELKVRVELLHNPAFCSLATTKRRHQQTVTI |
| | Sequence version 2 | PPKSSLSVPYVIVPLKTGLQEVEVKAAVYHHFISD |
| | (12 Dec. 2006); | GVRKSLKVVPEGIRMNKTVAVRTLDPERLGREGVQ |
| | residues 749-1663 | KEDIPPADLSDQVPDTESETRILLQGTPVAQMTED |
| | | AVDAERLKHLIVTPSGCGEQNMIGMTPTVIAVHYL |
| | | DETEQWEKFGLEKRQGALELIKKGYTQQLAFRQPS |
| | | SAFAAFVKRAPSTWLTAYVVKVFSLAVNLIAIDSQ |
| | | VLCGAVKWLILEKQKPDGVFQEDAPVIHQEMIGGL |
| | | RNNNEKDMALTAFVLISLQEAKDICEEQVNSLPGS |
| | | ITKAGDFLEANYMNLQRSYTVAIAGYALAQMGRLK |
| | | GPLLNKFLTTAKDKNRWEDPGKQLYNVEATSYALL |
| | | ALLQLKDFDFVPPVVRWLNEQRYYGGGYGSTQATF |
| | | MVFQALAQYQKDAPDHQELNLDVSLQLPSRSSKIT |
| | | HRIHWESASLLRSEETKENEGFTVTAEGKGQGTLS |
| | | VVTMYHAKAKDQLTCNKFDLKVTIKPAPETEKRPQ |
| | | DAKNTMILEICTRYRGDQDATMSILDISMMTGFAP |
| | | DTDDLKQLANGVDRYISKYELDKAFSDRNTLIIYL |
| | | DKVSHSEDDCLAFKVHQYFNVELIQPGAVKVYAYY |
| | | NLEESCTRFYHPEKEDGKLNKLCRDELCRCAEENC |
| | | FIQKSDDKVTLEERLDKACEPGVDYVYKTRLVKVQ |
| | | LSNDFDEYIMAIEQTIKSGSDE |
| | | VQVGQQRTFISPIKCREALKLEEKKHYLMWGLSSD |
| | | FWGEKPNLSYIIGKDTWVEHWPEEDECQDEENQKQ |
| | | CQDLGAFTESMVVFGCPN |
| 15 | Human C3α | SVQLTEKRMDKVGKYPKELRKCCEDGMRENPMRFS |
| | UniProt: P01024; | CQRRTRFISLGEACKKVFLDCCNYITELRRQHARA |
| | Entry version 242 | SHLGLAR |
| | (11 Dec. 2019) | |
| | Sequence version 2 | |
| | (12 Dec. 2006); | |
| | residues 672-748 | |
| 16 | Human C3b α′ chain | SNLDEDIIAEENIVSRSEFPESWLWNVEDLKEPPK |
| | fragment 1 | NGISTKLMNIFLKDSITTWEILAVSMSDKKGICVA |
| | UniProt: P01024; | DPFEVTVMQDFFIDLRLPYSVVRNEQVEIRAVLYN |
| | Entry version 242 | YRQNQELKVRVELLHNPAFCSLATTKRRHQQTVTI |
| | (11 Dec. 2019) | PPKSSLSVPYVIVPLKTGLQEVEVKAAVYHHFISD |
| | Sequence version 2 | GVRKSLKVVPEGIRMNKTVAVRTLDPERLGREGVQ |
| | (12 Dec. 2006); | KEDIPPADLSDQVPDTESETRILLQGTPVAQMTED |
| | residues 749-1663 | AVDAERLKHLIVTPSGCGEQNMIGMTPTVIAVHYL |
| | | DETEQWEKFGLEKRQGALELIKKGYTQQLAFRQPS |
| | | SAFAAFVKRAPSTWLTAYVVKVFSLAVNLIAIDSQ |
| | | VLCGAVKWLILEKQKPDGVFQEDAPVIHQEMIGGL |
| | | RNNNEKDMALTAFVLISLQEAKDICEEQVNSLPGS |
| | | ITKAGDFLEANYMNLQRSYTVAIAGYALAQMGRLK |
| | | GPLLNKFLTTAKDKNRWEDPGKQLYNVEATSYALL |
| | | ALLQLKDFDFVPPVVRWLNEQRYYGGGYGSTQATF |
| | | MVFQALAQYQKDAPDHQELNLDVSLQLPSRSSKIT |
| | | HRIHWESASLLRSEETKENEGFTVTAEGKGQGTLS |
| | | VVTMYHAKAKDQLTCNKFDLKVTIKPAPETEKRPQ |
| | | DAKNTMILEICTRYRGDQDATMSILDISMMTGFAP |
| | | DTDDLKQLANGVDRYISKYELDKAFSDRNTLIIYL |
| | | DKVSHSEDDCLAFKVHQYFNVELIQPGAVKVYAYY |
| | | NLEESCTRFYHPEKEDGKLNKLCRDELCRCAEENC |
| | | FIQKSDDKVTLEERLDKACEPGVDYVYKTRLVKVQ |
| | | LSNDFDEYIMAIEQTIKSGSDEVQVGQQRTFISPI |
| | | KCREALKLEEKKHYLMWGLSSDFWGEKPNLSYIIG |
| | | KDTWVEHWPEEDECQDEENQKQCQDLGAFTESMVV |
| | | FGCPN |
| 17 | Human C3 α′ chain | SEETKENEGFTVTAEGKGQGTLSVVTMYHAKAKDQ |
| | fragment 2 | LTCNKFDLKVTIKPAPETEKRPQDAKNTMILEICT |
| | UniProt: P01024; | RYRGDQDATMSILDISMMTGFAPDTDDLKQLANGV |
| | Entry version 242 | DRYISKYELDKAFSDRNTLIIYLDKVSHSEDDCLA |
| | (11 Dec. 2019) | FKVHQYFNVELIQPGAVKVYAYYNLEESCTRFYHP |
| | Sequence version 2 | EKEDGKLNKLCRDELCRCAEENCFIQKSDDKVTLE |
| | (12 Dec. 2006); | ERLDKACEPGVDYVYKTRLVKVQLSNDFDEYIMAI |
| | residues 1321-1663 | EQTIKSGSDEVQVGQQRTFISPIKCREALKLEEKK |
| | | HYLMWGLSSDFWGEKPNLSYIIGKDTWVEHWPEED |
| | | ECQDEENQKQCQDLGAFTESMVVFGCPN |
| 18 | C3f | SSKITHRIHWESASLLR |
| | UniProt: P01024; | |
| | Entry version 242 | |
| | (11 Dec. 2019) | |
| | Sequence version 2 | |
| | (12 Dec. 2006) | |
| | residues 1304-1320 | |
| 19 | Human C3c α′ chain | SNLDEDIIAEENIVSRSEFPESWLWNVEDLKEPPK |
| | fragment 1 | NGISTKLMNIFLKDSITTWEILAVSMSDKKGICVA |
| | UniProt: P01024; | DPFEVTVMQDFFIDLRLPYSVVRNEQVEIRAVLYN |
| | Entry version 242 | YRQNQELKVRVELLHNPAFCSLATTKRRHQQTVTI |
| | (11 Dec. 2019) | PPKSSLSVPYVIVPLKTGLQEVEVKAAVYHHFISD |
| | Sequence version 2 | GVRKSLKVVPEGIRMNKTVAVRTLDPERLGR |
| | (12 Dec. 2006); | |
| | residues 749-954 | |
| 20 | FH MS peptide | VTYKCFE |
| 21 | FHL-1 MS peptide | NGWSPTPRCIRVSFTL |
| 22 | FHR-1 MS peptide | ATFCDFPKINHGILYDEE |
| 23 | FHR-2 MS peptide 1 | RGWSTPPKCRSTISAE |
| 24 | FHR-2 MS peptide 2 | AMFCDFPKINHGILYDEE |
| 25 | FHR-3 MS peptide | VACHPGYGLPKAQTTVTCTE |
| 26 | FHR-4 MS peptide | YQCQSYYE |
| 27 | FHR-5 MS peptide | RGWSTPPICSFTKGE |
| 28 | C3.1 peptide | GTAFVIFGIQDGE |
| 29 | C3.2 peptide | LRRQHARASHLGLARSNLDE |
| 30 | C3.3 peptide | LRRQHARASHLGLAR |
| 31 | C3.4 peptide | LNLDVSLQLPSRSSKITHRIHWE |
| 32 | C3.5 peptide | LNLDVSLQLPSR |
| 33 | C3.6 peptide | RLGRE |
| 34 | C3.7 peptide | SSKITHRIHWE |
| 35 | C3.8 peptide | SASLLR |
| 36 | C3.9 peptide | RLGR |
| 37 | C3.10 peptide | HLIVTPSGCGE |
| 38 | FI peptide 1 | CAGTYDGSIDACKGDSGGPLVCMDANNVTYVWGVV |
| | | SWGE |
| 39 | FI peptide 2 | GTCVCKLPYQCPKNGTAVCATNRRSFPTYCQQKSL |
| | | E |
| 40 | FI peptide 3 | FPGVYTKVANYFDWISYHVGRPFISQYNV |
| 41 | FI peptide 4 | ANVACLDLGFQQGADTQRRFKLSDLSINSTE |
| 42 | FI peptide 5 | LPRSIPACVPWSPYLFQPNDTCIVSGWGRE |
| 43 | FI peptide 6 | KKCLAKKYTHLSCDKVFCQPWQRCIE |
| 44 | FI peptide 7 | LCCKACQGKGFHCKSGVCIPSQYQCNGE |
| 45 | FI peptide 8 | VKLVDQDKTMFICKSSWSMRE |
| 46 | FI peptide 9 | VKLISNCSKFYGNRFYE |
| 47 | FI peptide 10 | CLHPGTKFLNNGTCTAE |
| 48 | FI peptide 11 | NYNAGTYQNDIALIE |
| 49 | FI peptide 12 | GKFSVSLKHGNTDSE |
| 50 | FI peptide 13 | VGCAGFASVTQEE |
| 51 | FI peptide 14 | MKKDGNKKDCE |
| 52 | FI peptide 15 | YVDRIIFHE |
| 53 | FI peptide 16 | CLHVHCRGLE |
| 54 | FI peptide 17 | RVFSLQWGE |
| 55 | FI peptide 18 | ILTADMDAE |
| 56 | FI peptide 19 | KVTYTSQE |
| 57 | FI peptide 20 | VDCITGE |
| 58 | FI peptide 21 | NCGKPE |
| 59 | FI peptide 22 | TSLAE |
| 60 | FI peptide 23 | KDNE |
| 61 | C3.1.1 | AKIRYYTYLIMNKGRLLKAGRQVREPGQDLVVLPL |
| | | SITTDFIPSFRLVAYYTLIGASGQRE |
| 62 | C3.1.2 | RSGIPIVTSPYQIHFTKTPKYFKPGMPFDLMVFVT |
| | | NPDGSPAYRVPVAVQGE |
| 63 | C3.1.3 | KVVLVSLQSGYLFIQTDKTIYTPGSTVLYRIFTVN |
| | | HKLLPVGRTVMVNIE |
| 64 | C3.1.4 | DTVQSLTQGDGVAKLSINTHPSQKPLSITVRTKKQ |
| | | E |
| 65 | C3.1.5 | GDHGARVVLVAVDKGVFVLNKKNKLTQSKIWDVVE |
| 66 | C3.1.6 | KADIGCTPGSGKDYAGVFSDAGLTFTSSSGQQTAQ |
| | | RAE |
| 67 | C3.1.7 | QATRTMQALPYSTVGNSNNYLHLSVLRTE |
| 68 | C3.1.8 | AHDAQGDVPVTVTVHDFPGKKLVLSSE |
| 69 | C3.1.9 | GIPVKQDSLSSQNQLGVLPLSWDIPE |
| 70 | C3.1.10 | VVADSVWVDVKDSCVGSLVVKSGQSE |
| 71 | C3.1.11 | DLVGKSLYVSATVILHSGSDMVQAE |
| 72 | C3.1.12 | KTVLTPATNHMGNVTFTIPANRE |
| 73 | C3.1.13 | KGRNKFVTVQATFGTQVVE |
| 74 | C3.1.14 | VVLSRKVLLDGVQNPRAE |
| 75 | C3.1.15 | TLNVNFLLRMDRAHE |
| 76 | C3.1.16 | LVNMGQWKIRAYYE |
| 77 | C3.1.17 | SPMYSIITPNILRLE |
| 78 | C3.1.18 | DRQPVPGQQMTLKIE |
| 79 | C3.1.19 | VTITARFLYGKKVE |
| 80 | C3.1.20 | GTAFVIFGIQDGE |
| 81 | C3.1.21 | KFYYIYNE |
| 82 | C3.1.22 | NSPQQVFSTE |
| 83 | C3.1.23 | SLKRIPIE |
| 84 | C3.1.24 | YVLPSFE |
| 85 | C3.1.25 | QRISLPE |
| 86 | C3.1.26 | LQCPQPAA |
| 87 | C3.1.27 | VIVEPTE |
| 88 | C3.1.28 | TMVLE |
| 89 | C3.1.29 | LRPGE |
| 90 | C3.1.30 | FKSE |
| 91 | FHR-1 peptide 2 | NYNIALRWTAKQKLYLRTGE |
| 92 | FHR-2 peptide 3 | YNFVSPSKSFWTRITCAEE |
| 93 | FHR-3 peptide 2 | KGWSPTPRCIRVRTCSKSDIE |
| 94 | FHR-3 peptide 3 | NGYNQNYGRKFVQGNSTE |
| 95 | FHR-3 peptide 4 | QVKPCDFPDIKHGGLFHE |
| 96 | FHR-3 peptide 5 | FMCKLGYNANTSILSFQAVCRE |
| 97 | FHR-3 peptide 6 | YQCQPYYE |
| 98 | FHR-4 peptide 2 | NSRAKSNGMRFKLHDTLDYE |
| 99 | FHR-4 peptide 3 | DGWSHFPTCYNSSE |
| 100 | FHR-4 peptide 4 | ISYGNTTGSIVCGE |
| 101 | FHR-4 peptide 5 | FMCKLGYNANTSVLSFQAVCRE |
| 102 | FHR-5 peptide 2 | GTLCDFPKIHHGFLYDEE |
| 103 | FHR-5 peptide 3 | YAMIGNNMITCINGIWTE |
| 104 | FHR-5 peptide 4 | YGYVQPSVPPYQHGVSVE |
| 105 | FHR-5 peptide 5 | GDTVQIICNTGYSLQNNE |
| 106 | FHR-5 peptide 6 | IVCKDGRWQSLPRCVE |
| 107 | FHR-5 peptide 7 | DYNPFSQVPTGE |
| 108 | FHR-5 peptide 8 | QVKTCGYIPE |
| 109 | FHR-5 peptide 9 | ANVDAQPKKE |
| 110 | FHR-5 peptide 10 | WTTLPTCVE |
| 111 | FHR-5 peptide 11 | KVAVLCKE |
| 112 | FH peptide 2 | SNTGSTTGSIVCGYNGWSDLPICYE |
| 113 | FH peptide 3 | NGWSPTPRCIRVKTCSKSSIDIE |
| 114 | FH peptide 4 | LPKIDVHLVPDRKKDQYKVGE |
| 115 | FH peptide 5 | YYCNPRFLMKGPNKIQCVDGE |
| 116 | FH peptide 6 | NYNIALRWTAKQKLYSRTGE |
| 117 | FH peptide 7 | KWSHPPSCIKTDCLSLPSFE |
| 118 | FH peptide 8 | HGWAQLSSPPYYYGDSVE |
| 119 | FH peptide 9 | ISHGVVAHMSDSYQYGEE |
| 120 | FH peptide 10 | FDHNSNIRYRCRGKE |
| 121 | FH peptide 11 | ITCKDGRWQSIPLCVE |
| 122 | FH peptide 12 | GWIHTVCINGRWDPE |
| 123 | FH peptide 13 | KAKYQCKLGYVTADGE |
| 124 | FH peptide 14 | TTCYMGKWSSPPQCE |
| 125 | FH peptide 15 | SYAHGTKLSYTCE |
| 126 | FH peptide 16 | RVRYQCRSPYE |
| 127 | FH peptide 17 | GFGIDGPAIAKCLGE |
| 128 | FH peptide 18 | HGTINSSRSSQE |
| 129 | FH peptide 19 | YQCQNLYQLE |
| 130 | FH peptide 20 | WTTLPVCIVEE |
| 131 | FH peptide 21 | KIPCSQPPQIE |
| 132 | FH peptide 22 | SQYTYALKE |
| 133 | FH peptide 23 | QVQSCGPPPE |
| 134 | FH peptide 24 | KKDVYKAGE |
| 135 | FH peptide 25 | GLPCKSPPE |
| 136 | FH peptide 26 | KVSVLCQE |
| 137 | FH peptide 27 | HLKNKKE |
| 138 | FH peptide 28 | GGFRISEE |
| 139 | FH peptide 29 | LLNGNVKE |
| 140 | FH peptide 30 | YPTCAKR |
| 141 | FH peptide 31 | STCGDIPE |
| 142 | C3dg | EGVQKEDIPPADLSDQVPDTESETRILLQGTPVAQ |
| | UniProt: P01024; | MTEDAVDAERLKHLIVTPSGCGEQNMIGMTPTVIA |
| | Entry version 242 | VHYLDETEQWEKFGLEKRQGALELIKKGYTQQLAF |
| | (11 Dec. 2019) | RQPSSAFAAFVKRAPSTWLTAYVVKVFSLAVNLIA |
| | Sequence version 2 | IDSQVLCGAVKWLILEKQKPDGVFQEDAPVIHQEM |
| | (12 Dec. 2006) | IGGLRNNNEKDMALTAFVLISLQEAKDICEEQVNS |
| | residues 955-1303 | LPGSITKAGDFLEANYMNLQRSYTVAIAGYALAQM |
| | | GRLKGPLLNKFLTTAKDKNRWEDPGKQLYNVEATS |
| | | YALLALLQLKDFDFVPPVVRWLNEQRYYGGGYGST |
| | | QATFMVFQALAQYQKDAPDHQELNLDVSLQLPSR |
| 143 | C3g | EGVQKEDIPPADLSDQVPDTESETRILLQGTPVAQ |
| | UniProt: P01024; | MTEDAVDAERLK |
| | Entry version 242 | |
| | (11 Dec. 2019) | |
| | Sequence version 2 | |
| | (12 Dec. 2006) | |
| | residues 955-1001 | |
| 144 | C3d | HLIVTPSGCGEQNMIGMTPTVIAVHYLDETEQWEK |
| | UniProt: P01024; | FGLEKRQGALELIKKGYTQQLAFRQPSSAFAAFVK |
| | Entry version 242 | RAPSTWLTAYVVKVFSLAVNLIAIDSQVLCGAVKW |
| | (11 Dec. 2019) | LILEKQKPDGVFQEDAPVIHQEMIGGLRNNNEKDM |
| | Sequence version 2 | ALTAFVLISLQEAKDICEEQVNSLPGSITKAGDFL |
| | (12 Dec. 2006) | EANYMNLQRSYTVAIAGYALAQMGRLKGPLLNKFL |
| | residues 1002-1303 | TTAKDKNRWEDPGKQLYNVEATSYALLALLQLKDF |
| | | DFVPPVVRWLNEQRYYGGGYGSTQATFMVFQALAQ |
| | | YQKDAPDHQELNLDVSLQLPSR |
| 145 | Human Complement | GHCQAPDHFLFAKLKTQTNASDFPIGTSLKYECRP |
| | Receptor 1; | EYYGRPFSITCLDNLVWSSPKDVCKRKSCKTPPDP |
| | consensus | VNGMVHVITDIQVGSRINYSCTTGHRLIGHSSAEC |
| | sequence for CCPs | ILSGNX1AHWSTKPPICQRIPCGLPPTIANGDFIS |
| | 8-10, 15-17 | TNRENFHYGSVVTYRCNX2GSX3GRKVFELVGEPS |
| | (UniProt: P17927 | IYCTSNDDQVGIWSGPAPQCII |
| | residues 491 to 684; | |
| | residues 941 to | |
| | 1134) | |
| | Without leader | |
| | sequence | |
| 146 | Human Complement | GHCQAPDHFLFAKLKTQTNASDFPIGTSLKYECRP |
| | Receptor 1 CCPs 8- | EYYGRPFSITCLDNLVWSSPKDVCKRKSCKTPPDP |
| | 10 (UniProt: P17927 | VNGMVHVITDIQVGSRINYSCTTGHRLIGHSSAEC |
| | residues 491 to 684) | ILSGNAAHWSTKPPICQRIPCGLPPTIANGDFIST |
| | Without leader | NRENFHYGSVVTYRCNPGSGGRKVFELVGEPSIYC |
| | sequence | TSNDDQVGIWSGPAPQCII |
| 147 | Human Complement | GHCQAPDHFLFAKLKTQTNASDFPIGTSLKYECRP |
| | Receptor 1 CCPs | EYYGRPFSITCLDNLVWSSPKDVCKRKSCKTPPDP |
| | 15-17 (UniProt: | VNGMVHVITDIQVGSRINYSCTTGHRLIGHSSAEC |
| | P17927 residues | ILSGNTAHWSTKPPICQRIPCGLPPTIANGDFIST |
| | 941 to 1134) | NRENFHYGSVVTYRCNLGSRGRKVFELVGEPSIYC |
| | Without leader | TSNDDQVGIWSGPAPQCII |
| | sequence | |
| 148 | Human Complement | GHCQAPDHFLFAKLKTQTNASDFPIGTSLKYECRP |
| | Receptor 1 | EYYGRPFSITCLDNLVWSSPKDVCKRKSCKTPPDP |
| | CCPs 8-10 and 15- | VNGMVHVITDIQVGSRINYSCTTGHRLIGHSSAEC |
| | 17 | ILSGNAAHWSTKPPICQRIPCGLPPTIANGDFIST |
| | (contiguous; without | NRENFHYGSVVTYRCNPGSGGRKVFELVGEPSIYC |
| | leader sequence) | TSNDDQVGIWSGPAPQCIIGHCQAPDHFLFAKLKT |
| | +amino acid | QTNASDFPIGTSLKYECRPEYYGRPFSITCLDNLV |
| | differences between | WSSPKDVCKRKSCKTPPDPVNGMVHVITDIQVGSR |
| | CCPs 8-10 and 15- | INYSCTTGHRLIGHSSAECILSGNTAHWSTKPPIC |
| | 17 indicated with | QRIPCGLPPTIANGDFISTNRENFHYGSVVTYRCN |
| | wavy underline | LGSRGRKVFELVGEPSIYCTSNDDQVGIWSGPAPQ |
| | | CI- |
| 149 | Human | IVGGKRAQLGDLPWQVAIKDASGITCGGIYIGGCW |
| | Complement Factor | ILTAAHCLRASKTHRYQIWTTVVDWIHPDLKRIVI |
| | I proteolytic domain | EYVDRIIFHENYNAGTYQNDIALIEMKKDGNKKDC |
| | (UniProt: P05156 | ELPRSIPACVPWSPYLFQPNDTCIVSGWGREKDNE |
| | residues 340-574) | RVFSLQWGEVKLISNCSKFYGNRFYEKEMECAGTY |
| | | DGSIDACKGDSGGPLVCMDANNVTYVWGVVSWGEN |
| | | CGKPEFPGVYTKVANYFDWISYHVG |
| 150 | Human | EDCNELPPRRNTEILTGSWSDQTYPEGTQAIYKCR |
| | Complement Factor | PGYRSLGNVIMVCRKGEWVALNPLRKCQKRPCGHP |
| | H co-factor region | GDTPFGTFTLTGGNVFEYGVKAVYTCNEGYQLLGE |
| | (UniProt: P08603 | INYRECDTDGWTNDIPICEVVKCLPVTAPENGKIV |
| | residues 19-264) | SSAMEPDREYHFGQAVRFVCNSGYKIEGDEEMHCS |
| | | DDGFWSKEKPKCVEISCKSPDVINGSPISQKIIYK |
| | | ENERFQYKCNMGYEYSERGDAVCTESGWRPLPSCE |
| | | E |
| 151 | Human | GHCQAPDHFLFAKLKTQTNASDFPIGTSLKYECRP |
| | Complement | EYYGRPFSITCLDNLVWSSPKDVCKRKSCKTPPDP |
| | Receptor 1 CCPs | VNGMVHVITDIQVGSRINYSCTTGHRLIGHSSAEC |
| | 8-10 (UniProt: | ILSGNAAHWSTKPPICQRIPCGLPPTIANGDFIST |
| | P17927 residues | NRENFHYGSVVTYRCNPGSGGRKVFELVGEPSIYC |
| | 491 to 684) | TSNDDQVGIWSGPAPQCII |
| 152 | Human | GHCQAPDHFLFAKLKTQTNASDFPIGTSLKYECRP |
| | Complement | EYYGRPFSITCLDNLVWSSPKDVCKRKSCKTPPDP |
| | Receptor 1 CCPs | VNGMVHVITDIQVGSRINYSCTTGHRLIGHSSAEC |
| | 15-17 (UniProt: | ILSGNTAHWSTKPPICQRIPCGLPPTIANGDFIST |
| | P17927 residues | NRENFHYGSVVTYRCNLGSRGRKVFELVGEPSIYC |
| | 941 to 1134) | TSNDDQVGIWSGPAPQCII |
| 153 | GluC | MKGKFLKVSSLFVATLTTATLVSSPAANALSSKAM |
| | (Uniprot Q7A6A6 | DNHPQQTQSSKQQTPKIKKGGNLKPLEQREHANVI |
| | including N-terminal | LPNNDRHQITDTTNGHYAPVTYIQVEAPTGTFIAS |
| | signal peptide | GVVVGKDTLLTNKHVVDATHGDPHALKAFPSAINQ |
| | residues 1-29 and | DNYPNGGFTAEQITKYSGEGDLAIVKFSPNEQNKH |
| | propeptide residues | IGEVVKPATMSNNAETQVNQNITVTGYPGDKPVAT |
| | 30-68) | MWESKGKITYLKGEAMQYDLSTTGGNSGSPVFNEK |
| | | NEVIGIHWGGVPNEFNGAVFINENVRNFLKQNIED |
| | | IHFANDDQPNNPDNPDNPNNPDNPNNPDNPNNPDE |
| | | PNNPDNPNNPDNPDNGDNNNSDNPDAA |
| 154 | GluC (Uniprot | VILPNNDRHQITDTTNGHYAPVTYIQVEAPTGTFI |
| | Q7A6A6; without | ASGVVVGKDTLLTNKHVVDATHGDPHALKAFPSAI |
| | signal peptide or | NQDNYPNGGFTAEQITKYSGEGDLAIVKFSPNEQN |
| | propeptide) | KHIGEVVKPATMSNNAETQVNQNITVTGYPGDKPV |
| | | ATMWESKGKITYLKGEAMQYDLSTTGGNSGSPVFN |
| | | EKNEVIGIHWGGVPNEFNGAVFINENVRNFLKQNI |
| | | EDIHFANDDQPNNPDNPDNPNNPDNPNNPDNPNNP |
| | | DEPNNPDNPNNPDNPDNGDNNNSDNPDAA |
| 155 | FI peptide 24 | VGCAGFASVTQE |
| 156 | C3a-desArg | LRRQHARASHLGLA |
| 157 | C3f-desArg | SASLL |

### BRIEF DESCRIPTION OF THE FIGURES

FIG. 1. Schematic showing the C3 proteolytic cascade and the proteolytic events leading to the generation, breakdown and inactivation of C3b (modified from Maillard et al, J Am Soc Nephrol. 2015 Jul;26(7):1503-12). Proteoform-specific peptides for mass spectrometry are underlined.

FIG. 2. LC-SRM Trace showing detection of the heavy-labelled synthetic standard peptides of each individual RCA locus protein from a plasma sample.

FIG. 3. Linearity data for peptides derived from FH, FHL-1, and FHR1-5.

FIGS. 4A to 4D. Data confirming that C3 and C3 breakdown products in human plasma can be detected by MS with sufficient specificity and sensitivity. 4A: Total ion chromatograph from SRM-MS analysis showing specific and simultaneous detection of C3b fragment-specific peptides. 4B: Linearity data for seven of the ten peptides spiked into a plasma background. 4C: Coomassie-stained electrophoresis gel of C3 breakdown products obtained in vitro. 4D: MS quantification of key C3 fragments from the in vitro assay products shown in 4C.

### EXAMPLES

### Example 1: Generation of Peptides from Complement Proteins for Mass Spectrometry

GluC digestion was performed on FH, FHL-1, FHR1-5, FI, C3, C3b and C3b breakdown products to achieve distinct peptides for mass spectrometry. GluC digestion is described in Example 2.2.

Peptides that can be used to detect each protein or protein fragment are set out in Tables 1-4 below.

TABLE 1
Distinct FH family peptides
after GluC digestion.

| | | | SEQ |
|---|---|---|---|
| | | | ID |
| Protein | Peptide Sequence | Mass | No: |
| Factor H | VTYKCFE | 888.4051 | 20 |
| FHL1 | NGWSPTPRCIRVSFTL | 1832.9355 | 21 |
| FHR1 | ATFCDFPKINHGILYDEE | 2110.9669 | 22 |
| FHR2 | RGWSTPPKCRSTISAE | 1774.8784 | 23 |
| | and | | |
| | AMFCDFPKINHGILYDEE | 2140.9598 | 24 |
| FHR3 | VACHPGYGLPKAQTTVTCTE | 2074.9816 | 25 |
| FHR4 | YQCQSYYE | 1082.4015 | 26 |
| FHR5 | RGWSTPPICSFTKGE | 1664.7980 | 27 |

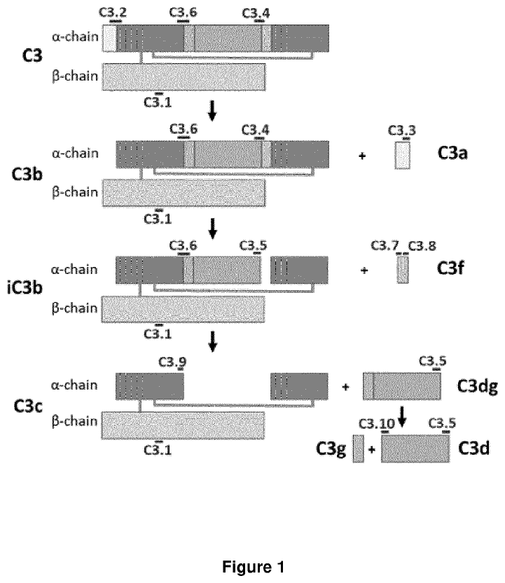

The series of proteolytic events leading to the generation, breakdown and inactivation of C3 are shown in FIG. 1. Proteoform-specific peptides produced by GluC digestion are underlined in FIG. 1 and are shown in Table 2. Table 3 shows how each protein can be detected individually using the peptides in Table 2.

TABLE 2
Peptide sequences for MS resulting from GluC
digestion of C3, C3b and breakdown products.

| | | | SEQ |
|---|---|---|---|
| | | Contained in | ID |
| Peptide | Peptide sequence | C3b products | No: |
| C3.1 | GTAFVIFGIQDGE | C3 + C3b + | 28 |
| | | iC3b + C3c | |
| C3.2 | LRRQHARASHLGLAR | C3 only | 29 |
| | SNLDE | | |
| C3.3 | LRRQHARASHLGLAR | C3a only | 30 |
| C3.4 | LNLDVSLQLPSRSSKI | C3 + C3b | 31 |
| | THRIHWE | | |
| C3.5 | LNLDVSLQLPSR | iC3b + C3dg + C3d | 32 |
| C3.6 | RLGRE | C3 + C3b + iC3b | 33 |
| C3.7 | SSKITHRIHWE | C3f | 34 |
| C3.8 | SASLLR | C3f | 35 |
| C3.9 | RLGR | C3c | 36 |
| C3.10 | HLIVTPSGCGE | C3d | 37 |

TABLE 3
Methodology for determining concentration of all C3/C3b
breakdown products using GluC digestion peptides of Table 2.

| Protein or fragment | Peptide(s) |
|---|---|
| C3 (total) | C3.1 |
| C3 only | C3.2 |
| C3a | C3.3 |
| C3b | C3.4 − C3.2 |
| iC3b | C3.6 − C3.4 |
| C3f | C3.7 or C3.8 |
| C3c | C3.9 |
| C3dg | C3.5 − C3.10 − iC3b [C3.6 − C3.4] |
| C3d | C3.10 |

TABLE 4
Alternative peptides for C3.1 resulting from
GluC digestion, to measure total C3 content.

| | | | | SED |
|---|---|---|---|---|
| | | | | ID |
| Peptide | Mass | Position | Peptide sequence | No. |
| C3.1.1 | 6957.8210 | 463-523 | AKIRYYTYLIMNKGRLLKAGRQVREPGQDLV | 61 |
| | | | VLPLSITTDFIPSFRLVAYYTLIGASGQRE | |
| C3.1.2 | 5778.9734 | 321-372 | RSGIPIVTSPYQIHFTKTPKYFKPGMPFDLMV | 62 |
| | | | FVTNPDGSPAYRVPVAVQGE | |
| C3.1.3 | 5650.1364 | 97-146 | KVVLVSLQSGYLFIQTDKTIYTPGSTVLYRIFT | 63 |
| | | | VNHKLLPVGRTVMVNIE | |
| C3.1.4 | 3904.1017 | 373-408 | DTVQSLTQGDGVAKLSINTHPSQKPLSITVRT | 64 |
| | | | KKQE | |
| C3.1.5 | 3861.1628 | 565-599 | GDHGARVVLVAVDKGVFVLNKKNKLTQSKI | 65 |
| | | | WDVVE | |
| C3.1.6 | 3807.7645 | 600-637 | KADIGCTPGSGKDYAGVFSDAGLTFTSSSG | 66 |
| | | | QQTAQRAE | |
| C3.1.7 | 3263.6357 | 414-442 | QATRTMQALPYSTVGNSNNYLHLSVLRTE | 67 |
| C3.1.8 | 2845.4609 | 24-50 | AHDAQGDVPVTVTVHDFPGKKLVLSSE | 68 |
| C3.1.9 | 2819.4705 | 150-175 | GIPVKQDSLSSQNQLGVLPLSWDIPE | 69 |
| C3.1.10 | 2691.3425 | 524-549 | VVADSVWVDVKDSCVGSLVVKSGQSE | 70 |
| C3.1.11 | 2618.3261 | 296-320 | DLVGKSLYVSATVILHSGSDMVQAE | 71 |
| C3.1.12 | 2511.2904 | 51-73 | KTVLTPATNHMGNVTFTIPANRE | 72 |
| C3.1.13 | 2108.1378 | 78-96 | KGRNKFVTVQATFGTQVVE | 73 |
| C3.1.14 | 1992.1480 | 278-295 | VVLSRKVLLDGVQNPRAE | 74 |
| C3.1.15 | 1827.9414 | 448-462 | TLNVNFLLRMDRAHE | 75 |
| C3.1.16 | 1769.8923 | 176-189 | LVNMGQWKIRAYYE | 76 |
| C3.1.17 | 1745.9386 | 1-15 | SPMYSIITPNILRLE | 77 |
| C3.1.18 | 1738.9036 | 550-564 | DRQPVPGQQMTLKIE | 78 |
| C3.1.19 | 1623.9348 | 231-244 | VTITARFLYGKKVE | 79 |
| C3.1.20 | 1352.6612 | 245-257 | GTAFVIFGIQDGE | 80 |
| C3.1.21 | 1138.5335 | 219-226 | KFYYIYNE | 81 |
| C3.1.22 | 1135.5146 | 190-199 | NSPQQVFSTE | 82 |
| C3.1.23 | 954.5862 | 265-272 | SLKRIPIE | 83 |
| C3.1.24 | 853.4221 | 205-211 | YVLPSFE | 84 |
| C3.1.25 | 841.4657 | 258-264 | QRISLPE | 85 |
| C3.1.26 | 826.4007 | 638-645 | LQCPQPAA | 86 |
| C3.1.27 | 785.4171 | 212-218 | VIVEPTE | 87 |
| C3.1.28 | 591.2938 | 19-23 | TMVLE | 88 |
| C3.1.29 | 570.3125 | 443-447 | LRPGE | 89 |
| C3.1.30 | 509.2485 | 74-77 | FKSE | 90 |

GluC digestion of Factor I (FI) produced the candidate peptides in Table 5 for MS analysis. SEQ ID NO:45 to 56 and 155 contain 8-21 amino acids and are a good length for MS analysis.

TABLE 5
Peptide sequences resulting from GluC digestion of Fl.

| | | | | SEQ |
|---|---|---|---|---|
| Peptide | Mass | Position | Peptide sequence | ID NO: |
| 1 | 3996.7183 | 510-548 | CAGTYDGSIDACKGDSGGPLVCMDANNVTYVWGVVS | 38 |
| | | | WGE | |
| 2 | 3994.8853 | 57-92 | GTCVCKLPYQCPKNGTAVCATNRRSFPTYCQQKSLE | 39 |
| 3 | 3467.7211 | 555-583 | FPGVYTKVANYFDWISYHVGRPFISQYNV | 40 |
| 4 | 3397.6804 | 150-180 | ANVACLDLGFQQGADTQRRFKLSDLSINSTE | 41 |
| 5 | 3388.6605 | 446-475 | LPRSIPACVPWSPYLFQPNDTCIVSGWGRE | 42 |
| 6 | 3155.5773 | 31-56 | KKCLAKKYTHLSCDKVFCQPWQRCIE | 43 |
| 7 | 2991.2861 | 254-281 | LCCKACQGKGFHCKSGVCIPSQYQCNGE | 44 |
| 8 | 2531.2455 | 129-149 | VKLVDQDKTMFICKSSWSMRE | 45 |
| 9 | 2068.0320 | 489-505 | VKLISNCSKFYGNRFYE | 46 |
| 10 | 1805.8309 | 93-109 | CLHPGTKFLNNGTCTAE | 47 |
| 11 | 1698.7969 | 420-434 | NYNAGTYQNDIALIE | 48 |
| 12 | 1605.7867 | 110-124 | GKFSVSLKHGNTDSE | 49 |
| 13 | 1297.5729 | 291-303 | VGCAGFASVTQEE | 50 |
| 14 | 1168.272 | 291-302 | VGCAGFASVTQE | 155 |
| 15 | 1295.6082 | 435-445 | MKKDGNKKDCE | 51 |
| 16 | 1191.6156 | 411-419 | YVDRIIFHE | 52 |
| 17 | 1166.5557 | 181-190 | CLHVHCRGLE | 53 |
| 18 | 1121.5738 | 480-488 | RVFSLQWGE | 54 |
| 19 | 978.4448 | 306-314 | ILTADMDAE | 55 |
| 20 | 955.4731 | 19-26 | KVTYTSQE | 56 |
| 21 | 736.3182 | 282-288 | VDCITGE | 57 |
| 22 | 647.2817 | 549-554 | NCGKPE | 58 |
| 23 | 520.2613 | 191-195 | TSLAE | 59 |
| 24 | 505.2252 | 476-479 | KDNE | 60 |

### Example 2: Mass Spectrometry

### 2.1 Preparation of Stable Isotopic Standards (SIS) Spiking Solution

High purity heavy-labelled synthetic standards, with S-carboxymethylated (CAM) cysteine residues, were obtained (Cambridge Research Biochemicals, Cambridge, UK) and diluted to 1 μg/μL with 50:50 acetonitrile:water +0.1% formic acid (Table 6).

A mixed SIS solution was prepared by firstly diluting stock solution of FHL-1, FHR1, FHR2, FHR3, FHR4 and FHR5 by tenfold (no dilution of CFH stock was required), then adding the appropriate amounts of each individual diluted solution to a final volume of 200 μL in 0.1% TFA. This was then stored at −80° C. in 5 μL aliquots for further dilution immediately prior to spiking.

Spiking solution was prepared immediately prior to sample addition by adding 195 μL 50:50 acetonitrile:water to a 5 μL aliquot of the mixed SIS solution. 2 μL of this was carefully added to each digested sample prior to drying down.

TABLE 6
Stock solutions of stable isotopic standards (SIS)
at 1 μg/MI. The residues in bold were chosen to
carry a stable-heavy isotype to enable quantitation.
Lower case ‘c’ denotes a S-carboxymethylated (CAM)
cysteine residue. Residue in bold type contained
an isotopically heavy amino acid, where K(+8),
R(+10), F(+10) and Y(+10).

| | | | | Net | Solvent | Conc, |
|---|---|---|---|---|---|---|
| | Peptide | MW, | Purity, | Content, | Volume, | pmol/ |
| Protein | Sequence | g | % | % | μL | μL |
| CFH | VTYKcFE | 953.4 | 98.6 | 72.6 | 716 | 1050 |
| FHL-1 | NGWSPTPRc | 1900 | 97.4 | 78.9 | 768 | 526.3 |
| | IRVSFTL | | | | | |
| FHR1 | ATFcDFPKI | 2178 | 100.0 | 80.7 | 807 | 459.1 |
| | NHGILYDEE | | | | | |
| FHR2 | RGWSTPPKE | 1845.9 | 100.0 | 55.5 | 555 | 541.7 |
| | RSTISAE | | | | | |
| FHR3 | VAcHPGYGL | 2207.1 | 98.4 | 75.0 | 738 | 453.1 |
| | PKAQTTVT | | | | | |
| | CTE | | | | | |
| FHR4 | YQcQSYYE | 1149.7 | 97.6 | 84.1 | 821 | 869.8 |
| FHR5 | RGWSTPPI | 1739.8 | 95.6 | 74.0 | 707 | 574.8 |
| | CSFTKGE | | | | | |

### 2.2 Preparation of Samples for Analysis by LC-MS/MS

Frozen plasma samples were allowed to thaw to room temperature before being vortexed hard for 5 minutes to dissolve any soluble material, then centrifuged at 13,300 g for 30 min to settle any insoluble material.

To a 5 μL plasma aliquot (equivalent to approximately 350 μL protein), 90 μL of 50 mM ammonium bicarbonate (pH 7.8), 2 μL of ProteaseMAX™ (Promega, Southampton, UK) solution (1% w/v in 50 mM ammonium bicarbonate) and 1 μL of 500 mM dithiothreitol prepared in 50 mM ammonium bicarbonate was added. This was vortexed briefly to mix, then given a pulse spin before incubating at 56° C. for 25 min.

After cooling to room temperature, 3 μL 500 mM iodoacetamide (prepared in 50 mM ammonium bicarbonate) was added. This was vortexed briefly to mix, then given a pulse spin before incubating at room temperature and in the dark for 15 min.

A further 1 μL of ProteaseMAX solution (1% w/v in 50 mM ammonium bicarbonate) and 5 μL of 1 μg/uL endoproteinase GluC (Roche, Mannheim, Germany) were added. The mixture was vortexed briefly, then given a pulse spin before incubating for 16 hours at 25° C. with slight shaking (400 rpm).

To the digested peptide mix obtained, 6 μL 10% v/v trifluoroacetic acid (TFA) and 2 μL of SIS spiking solution were added, vortexed briefly to mix, then pulse spin. The solution was placed into an evaporator and dried. Finally the peptides were reconstituted in 50 μL 0.1% TFA and vortexed to dissolve any residue before centrifuging at 13,300 g for 30 min to settle any insoluble/particulate material. Approximately 48 μL (taking care to leave behind any precipitated material) was transferred to a LC autosampler vial for subsequent analysis by LC-MS/MS.

### 2.3 LC-SRM/MS Analysis of Plasma Digests

SRM analyses of plasma digests were performed on a 6495 triple quadrupole mass spectrometer with iFunnel-equipped electrospray ion source (Agilent, Santa Clara, CA, USA) coupled to an Infinity 1200 Series liquid chromatography system consisting of 1290 autosampler, 1260 Quat Pump VL pump and TCC column oven modules (Agilent, Santa Clara, CA, USA). Samples were injected directly (4 μL) onto a C18 column (250 mm×2.1 mm i.d., Thermo Scientific Acclaim 120, 3 μm particle size) that was maintained at a column temperature of 50° C. Compounds were developed using a gradient elution of increasing acetonitrile concentration with Buffer A consisting of Water +0.1% formic acid and Buffer B being Acetonitrile +0.1% formic acid. The flow rate was maintained at 250 μL/min with an initial composition of 5% Buffer B.

The following gradient elution profile was used to separate the peptides (time: % B): 0 min: 5% B; 2 min: 5% B; 3 min: 12% B; 12 min: 15% B; 15 min: 20% B; 30 min: 25% B; 31 min: 90% B; 39 min: 90% B; 40 min: 5% B; 49 min: 5% B.

Optimized SRM settings were determined using SIS solutions and are provided in Table 7.

TABLE 7
SRM transitions and optimal collision energies
for FH family peptides (Quantitation ions in
bold).

| | | Precursor | Product | Collision |
|---|---|---|---|---|
| | Peptide | ion | ions | energy, |
| Protein | Sequence | m/z | m/z | eV |
| CFH | VTYKcFE | 473.7 | 583.3, | 16, 16, |
| | (Light) | | 847.4, | 16 |
| | | | 746.3 | |
| CFH | VTYKcFE | 477.7 | 591.3, | 16, 16, |
| | (Heavy) | | 855.4, | 16 |
| | | | 754.3 | |
| FHL-1 | NGWSPTPR | 631.2 | 723.9, | 19, 19, |
| | CIRVSFTL | | 860.5, | 19 |
| | (Light) | | 767.4 | |
| FHL-1 | NGWSPTPR | 634.3 | 728.9, | 19, 19, |
| | cIRVSFTL | | 865.5, | 19 |
| | (Heavy) | | 772.4 | |
| FHR1 | ATFcDFPK | 724.2 | 925.6, | 20, 16, |
| | INHGILYD | | 1011.9, | 20 |
| | EE | | | |
| | (Light) | | 947.1 | |
| FHR1 | ATFcDFPK | 727.2 | 930.6, | 20, 16, |
| | INHGILYD | | 1016.9, | 20 |
| | EE | | | |
| | (Heavy) | | 952.1 | |
| FHR2 | RGWSTPPK | 459.1, | 539.2, | 8, 26 |
| | ERSTISAE | 611.8 | 798.9 | |
| | (Light) | | | |
| FHR2 | RGWSTPPK | 462.6, | 543.9, | 8, 26 |
| | cRSTISAE | 616.3 | 806.0 | |
| | (Heavy) | | | |
| FHR3 | VACHPGYG | 730.7 | 1022.4, | 16, 18 |
| | LPKAQTTV | | 971.7 | |
| | TcTE | | | |
| | (Light) | | | |
| FHR3 | VACHPGYG | 736.7 | 1031.4, | 16, 18 |
| | LPKAQTTV | | 980.7 | |
| | TCTE | | | |
| | (Heavy) | | | |
| FHR4 | YQcQSYYE | 570.7 | 830.3, | 11, 10, |
| | (Light) | | 993.1, | 14 |
| | | | 311.1 | |
| FHR4 | YQcQSYYE | 575.7 | 840.3, | 11, 10, |
| | (Heavy) | | 1003.1, | 14 |
| | | | 311.1 | |
| FHR5 | RGWSTPPI | 575.2 | 828.4, | 16, 15, |
| | CSFTKGE | | 895.5, | 20 |
| | (Light) | | 588.3 | |
| FHR5 | RGWSTPPI | 581.2 | 836.4, | 16, 15, |
| | CSFTKGE | | 905.5, | 20 |
| | (Heavy) | | 598.3 | |

TABLE 8
Peptides and transitions for quantitation
of C3/C3b breakdown products.

| | Se- | Contained | |
|---|---|---|---|
| Peptide | quence | in . . . | Transitions |
| C3.1 | GTAFVIF | C3 + C3b + | 569.0/483.2 |
| | GIQDGE | iC3b + C3c | 569.0/654.3 |
| | | | 569.0/801.4 |
| C3.2 | LRRQHAR | C3 only | 384.2/431.6 |
| | ASHLGLA | | 384.2/510.4 |
| | RSNLDE | | 461.0/503.3 |
| C3.3 | LRRQHARA | C3a only | 291.2/304.3 |
| | SHLGLAR | | 291.2/395.8 |
| | | | 436.3/598.7 |
| C3.4 | LNLDVSL | C3 + C3b | 546.8/578.7 |
| | QLPSRSS | | 683.2/797.1 |
| | KITHRIH | | 683.2/834.9 |
| | WE | | |
| C3.5 | LNLDVSL | iC3b + C3dg + | 677.9/1014.4 |
| | QLPSR | C3d | 677.9/359.1 |
| | | | 677.9/800.2 |
| C3.6 | RLGRE | C3 + C3b + | 251.3/175.1 |
| | | iC3b | 251.3/232.1 |
| | | | 251.3/345.2 |
| C3.7 | SSKITHRI | C3f | 349.3/354.2 |
| | HWE | | 349.3/436.4 |
| | | | 349.3/489.8 |
| C3.8 | SASLLR | C3f | 323.7/159.1 |
| | | | 323.7/288.2 |
| | | | 323.7/488.3 |
| C3.9 | RLGR | C3c | 251.3/175.1 |
| | | | 251.3/232.1 |
| | | | 251.3/345.2 |
| C3.10 | HLIVTPSG | C3d | 585.3/251.1 |
| | CGE | | 585.3/394.5 |
| | | | 585.3/404 |

In order to protect the source region from unwanted contaminants, a switching valve located between the column and source was diverted to the waste position at points in the chromatogram when the analyte peptides were not eluting. This allowed for six windows (two of the peptides, FHR-2 and FHL-1, eluted within the same window) of acquisition, of approximately one minute each, to be acquired with the column on-line to the mass spectrometer.

### 2.4 Results

### FH Family Proteins

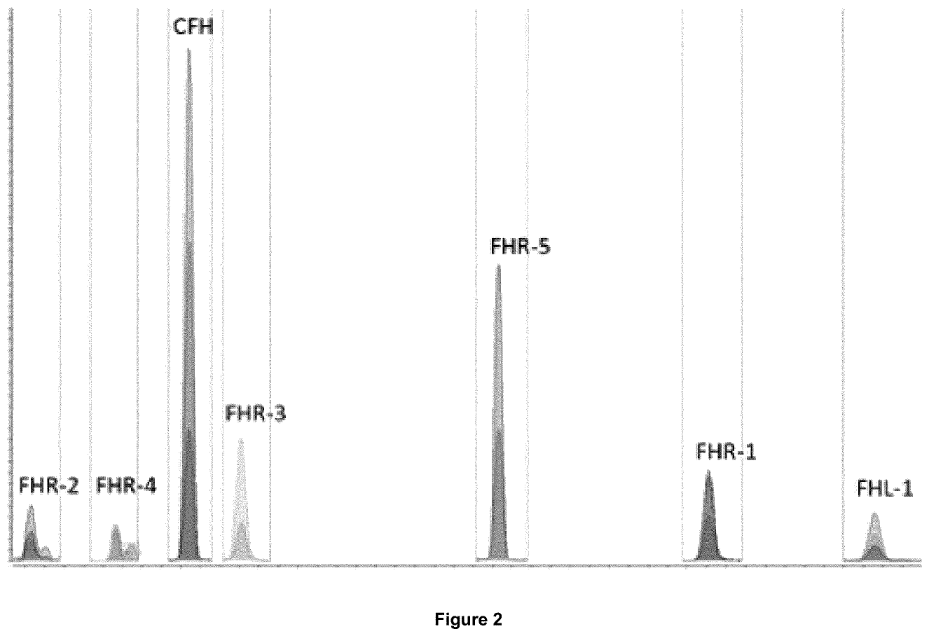

FIG. 2 shows a LC-SRM Trace showing detection of the heavy-labelled synthetic standards of each individual RCA locus protein from a plasma sample. This demonstrates that the method is feasible, specific and has the required sensitivity to distinguish between peptides from these seven proteins, in particular between FH and FHL-1.

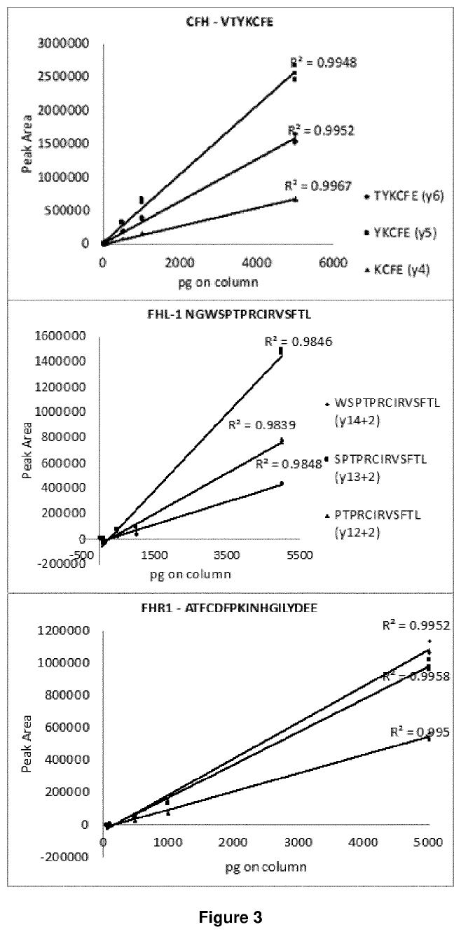

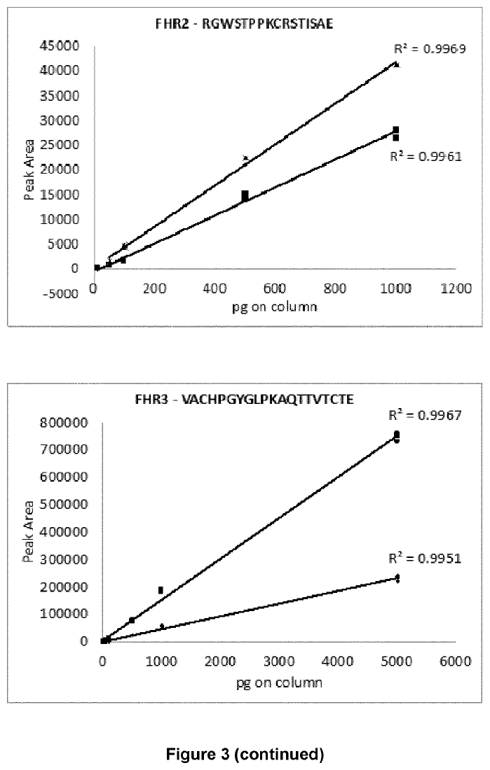

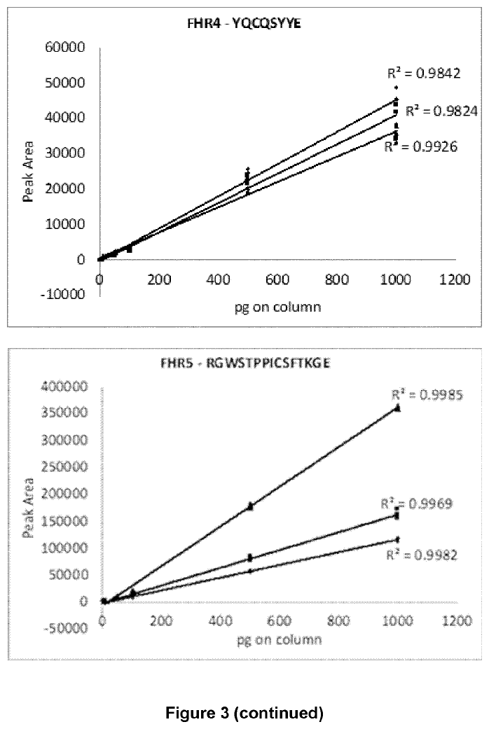

FIG. 3 shows linearity data for FH, FHL-1, and FHR1-5. This demonstrates that the GluC digestion produces peptides that can be detected individually and specifically in native serum at endogenous levels. It also shows that the assay is capable of quantifying the level of each protein in the sample. Increasing amounts of protein increase the signal in a predictable manner, allowing determination of the levels, as well as the presence, of each of the proteins. Also demonstrated is that the assay is free from interference.

Lower limits of quantitation were defined as plasma concentrations of FH=25 nM, FHL-1=0.25 nM, FHR-1=2 nM, FHR-2=1 nM, FHR-3=1 nM, FHR-4=4 nM and FHR-5=3 nM.

### C3 and C3 Breakdown Products

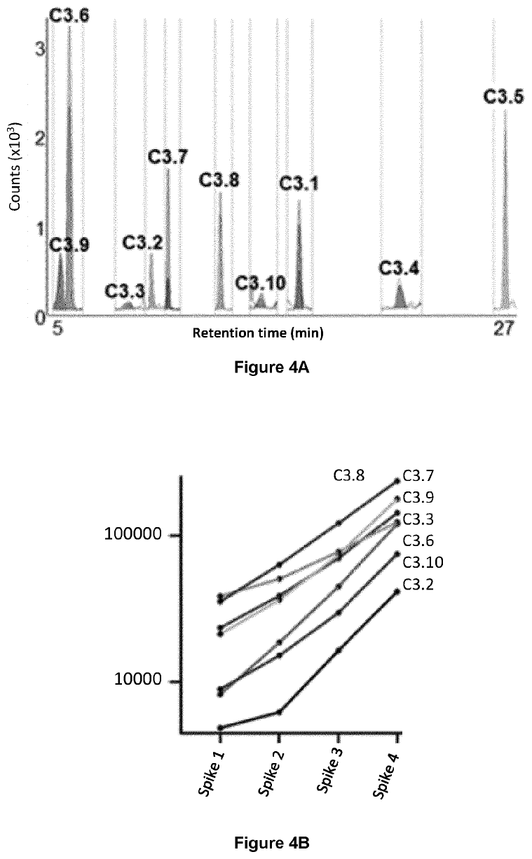

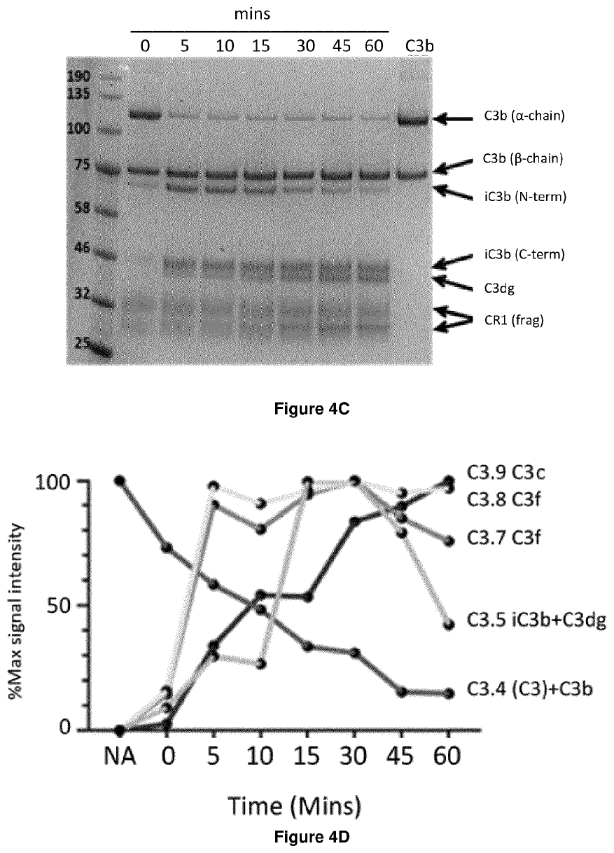

Synthetic versions of the peptides in Table 2 were synthesised to confirm and optimise their detection by MS to confirm that they could be quantified in a linear manner, and to demonstrate that they could be detected at endogenous levels in a serum or plasma sample. This is shown by FIGS. 4A to 4D.

FIG. 4A shows that all peptides in Table 2 can be detected individually in a plasma sample by SRM-MS using at least three transitions. The specificity of the assay for the peptides of interest is confirmed by the relative intensities of the transitions matching the relative intensity of the relevant product ions in an MS/MS scan. FIG. 4B confirms the specificity of the peptides, showing experiments in which the plasma sample was spiked with crude synthetic peptide which demonstrated the appropriate increase in signal.

C3b breakdown was further analysed in an in vitro assay. C3b was incubated along with FI and a fragment of cofactor CR1, selected over FH as CR1 drives the reaction to cleavage of iC3b to C3c+C3dg, whereas FH will only support cleavage of C3b to iC3b. Sequential samples were taken from the reaction and stopped by boiling.

FIG. 4C shows the time course of the C3b breakdown via gel electrophoresis. Analysis using MS and the peptides of Table 2 demonstrates that the formation of C3b fragments iC3b, C3f and C3c, and loss of intact C3b can be clearly detected over time (FIG. 4D). Not all peptides are shown since some (e.g. C3a) will not be present in the in vitro set-up, and others represent multiple products.

These data demonstrate that C3/C3b breakdown can be measured in a quantitative manner using GluC-derived peptides and MS. This enables the presence and levels of complement proteins to be detected in complement-related diseases such as AMD, as well as providing information as to successful treatment outcomes.

A single assay which can measure all FH family, C3 fragments and FI proteins allows for the simultaneous analysis of all key proteins in the complement amplification loop from just one sample and with efficient throughput.

1. A method for determining the level of at least one complement protein in a sample, the method comprising:
digesting the protein(s) with endoproteinase GluC to obtain one or more peptides; and
determining the level of the one or more peptides by mass spectrometry.

2.-3. (canceled)

4. A method according to claim 1, wherein the method comprises determining the concentration of the complement protein(s) in the sample.

5. (canceled)

6. A method according to claim 1, wherein the complement protein(s) is one or more of FH, FHL-1, FHR1, FHR2, FHR3, FHR4, and/or FHR5.

7. (canceled)

8. A method according to claim 1, wherein the complement protein(s) is involved in the complement amplification loop and/or C3 convertase activity.

9. A method according to claim 1, wherein the complement protein(s) is a breakdown product of C3b.

10. A method according to claim 1, wherein the complement protein(s) is one or more of C3, C3b, C3a, iC3b, C3f, C3c, C3dg, and/or C3d.

11. (canceled)

12. A method according to claim 1, wherein the complement protein is FI.

13. A method according to claim 1, wherein the sample has been obtained from a subject, optionally wherein the sample comprises, or is derived from, blood, lymph, plasma, serum, tissue, or cells.

14. A method according to claim 1, wherein the method comprises a step of obtaining the sample from a subject, optionally wherein the sample comprises, or is derived from, blood, lymph, plasma, serum, tissue, or cells.

15. (canceled)

16. A method according to claim 1, wherein the one or more peptides are selected from the group consisting of:
(a)
(SEQ ID NO: 20)
VTYKCFE;
(b)
(SEQ ID NO: 21)
NGWSPTPRCIRVSFTL;
(c)
(SEQ ID NO: 22)
ATFCDFPKINHGILYDEE;
(d)
(SEQ ID NO: 23)
RGWSTPPKCRSTISAE
or
(SEQ ID NO: 24)
AMFCDFPKINHGILYDEE;
(e)
(SEQ ID NO: 25)
VACHPGYGLPKAQTTVTCTE;
(f)
(SEQ ID NO: 26)
YQCQSYYE;
(g)
(SEQ ID NO: 27)
RGWSTPPICSFTKGE;
(h)
(SEQ ID NO: 28)
GTAFVIFGIQDGE;
(i)
(SEQ ID NO: 29)
LRRQHARASHLGLARSNLDE;
(j)
(SEQ ID NO: 30)
LRRQHARASHLGLAR
and/or
(SEQ ID NO: 156)
LRRQHARASHLGLA;
(k)
(SEQ ID NO: 31)
LNLDVSLQLPSRSSKITHRIHWE;
(l)
(SEQ ID NO: 32)
LNLDVSLQLPSR;
(m)
(SEQ ID NO: 33)
RLGRE;
(n)
(SEQ ID NO: 34)
SSKITHRIHWE;
(o)
(SEQ ID NO: 35)
SASLLR
and/or
(SEQ ID NO: 157)
SASLL;
(p)
(SEQ ID NO: 36)
RLGR;
(q)
(SEQ ID NO: 37)
HLIVTPSGCGE;
and/or
(r)
any one or more of SEQ ID NO: 38 to 60.

17.-18. (canceled)

19. A method of treating a complement-related disorder in a subject, the method comprising:
(a) digesting at least one complement protein in a sample obtained from the subject with endoproteinase GluC to obtain one or more peptides;
(b) determining the presence and/or level of the one or more peptides by mass spectrometry;
(c) using the results of (b) to determine whether the subject has an increase in the level of one or more of C3, C3b, C3a, iC3b, FHR1, FHR2, FHR3, FHR4, and/or FHR5; and/or a decrease in the level of one or more of iC3b, C3f, C3c, C3dg, C3d, C3g, FI, FH, FHL-1, FHR1, FHR2, FHR3, FHR4, and/or FHR5, as compared to reference value(s) obtained previously from the same subject or obtained from a subject without a complement-related disorder; and
(d) treating the subject with a therapeutically effective amount of a complement-targeted therapeutic.

20.-21. (canceled)

22. A method of treating a complement-related disorder in a subject, the method comprising:
(a) digesting at least one complement protein in a sample obtained from the subject with endoproteinase GluC to obtain one or more peptides;
(b) determining the presence and level of the one or more peptides by mass spectrometry, wherein the subject has an increase in the level of one or more of C3, C3b, C3a, iC3b, FHR1, FHR2, FHR3, FHR4, and/or FHR5; and/or a decrease in the level of one or more of iC3b, C3f, C3c, C3dg, C3d, C3g, FI, FH, FHL-1, FHR1, FHR2, FHR3, FHR4, and/or FHR5, as compared to reference value(s) obtained previously from the same subject or obtained from a subject without a complement related disorder; and
based on the results of (b), administering a therapeutically effective amount of a complement-targeted therapeutic.

23. A method according to claim 22, wherein the method comprises obtaining a sample from a subject comprising at least one complement protein, optionally wherein the sample comprises or is derived from blood, lymph, plasma, serum, tissue, or cells.

24. A method according to claim 22, wherein the complement-related disorder is selected from macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schonlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, neurodegeneration/neurodegenerative disease, dementia, multiple sclerosis (MS), cancer, stroke, Parkinson's disease, and/or Alzheimer's disease.

25-26. (canceled)

27. The method according to claim 22, wherein the subject has an increase in the level of one or more of C3, C3b, C3a, iC3b, FHR1, FHR2, FHR3, FHR4, and/or FHR5 as compared to reference value(s) obtained previously from the same subject or obtained from a subject without a complement related disorder, and the complement targeted therapeutic is an agent that reduces the level of the one or more of C3, C3b, C3a, iC3b, FHR1, FHR2, FHR3, FHR4, and/or FHR5 that are increased as compared to the reference value(s).

28. The method according to claim 22, wherein the subject has a decrease in the level of one or more of iC3b, C3f, C3c, C3dg, C3d, C3g, FI, FH, FHL-1, FHR1, FHR2, FHR3, FHR4, and/or FHR5 as compared to reference value(s) obtained previously from the same subject or obtained from a subject without a complement related disorder, and the complement targeted therapeutic is an agent that increases the level of the one or more of iC3b, C3f, C3c, C3dg, C3d, C3g, FI, FH, FHL-1, FHR1, FHR2, FHR3, FHR4, and/or FHR5 that are decreased as compared to the reference value(s).

29. The method according to claim 19, wherein the method comprises obtaining a sample from a subject comprising at least one complement protein, optionally wherein the sample comprises or is derived from blood, lymph, plasma, serum, tissue, or cells.

30. The method according to claim 19, wherein the subject has or is determined to have a complement-related disorder selected from macular degeneration, age related macular degeneration (AMD), geographic atrophy (‘dry’ (i.e. non-exudative) AMD), early AMD, early onset macular degeneration (EOMD), intermediate AMD, late/advanced AMD, ‘wet’ (neovascular or exudative) AMD, choroidal neovascularisation (CNV), retinal dystrophy, Haemolytic Uremic Syndrome (HUS), atypical Haemolytic Uremic Syndrome (aHUS), DEAP HUS (Deficiency of FHR plasma proteins and Autoantibody Positive form of Hemolytic Uremic Syndrome), autoimmune uveitis, Membranoproliferative Glomerulonephritis Type II (MPGN II), sepsis, Henoch-Schönlein purpura (HSP), IgA nephropathy, chronic kidney disease, paroxysmal nocturnal hemoglobinuria (PNH), autoimmune hemolytic anemia (AIHA), systemic lupus erythematosis (SLE), Sjogren's syndrome (SS), rheumatoid arthritis (RA), C3 glomerulopathy (C3G), dense deposit disease (DDD), C3 nephritic factor glomerulonephritis (C3 NF GN), FHR5 nephropathy, hereditary angioedema (HAE), acquired angioedema (AAE), encephalomyelitis, atherosclerosis, neurodegeneration/neurodegenerative disease, dementia, multiple sclerosis (MS), cancer, stroke, Parkinson's disease, and/or Alzheimer's disease.

31. The method according to claim 19, wherein the subject has an increase in the level of one or more of C3, C3b, C3a, iC3b, FHR1, FHR2, FHR3, FHR4, and/or FHR5 as compared to reference value(s) obtained previously from the same subject or obtained from a subject without a complement related disorder, and the complement-targeted therapeutic is an agent that reduces the level of the one or more of C3, C3b, C3a, iC3b, FHR1, FHR2, FHR3, FHR4, and/or FHR5 that are increased as compared to the reference value(s).

32. The method according to claim 19, wherein the subject has a decrease in the level of one or more of iC3b, C3f, C3c, C3dg, C3d, C3g, FI, FH, FHL-1, FHR1, FHR2, FHR3, FHR4, and/or FHR5 as compared to reference value(s) obtained previously from the same subject or obtained from a subject without a complement related disorder, and the complement-targeted therapeutic is an agent that increases the level of the one or more of iC3b, C3f, C3c, C3dg, C3d, C3g, FI, FH, FHL-1, FHR1, FHR2, FHR3, FHR4, and/or FHR5 that are decreased as compared to the reference value(s).
